SEQUENCE LISTING (71 sequences)

SEQ ID NO: 1 - protein, 619 aa, Glycine max
MMRTRFPLLLLGVVFLASVSVSFGIAYWEKQNPSHNKCLRSCNSEKDSYRNQACHARCNLLKVEEEEECEEGQPRPRPQHPERERQQHGEKEEDEGEQPRPFPFPRPRQPHQEEEHEQKEEHEWHRKEEKHGGKGSEEEQDEREHPRPHQPHQKEEEKHEWQHKQEKHQGKESEEEEEDQDEDEEQDKESQESEGSESQREPRRHKNKNPFHFNSKRFQTLFKNQYGHVRVLQRFNKRSQQLQNLRDYRILEFNSKPNTLLLPHHADADYLIVILNGTAILTLVNNDDRDSYNLQSGDALRVPAGTTYYVVNPDNDENLRMITLAIPVNKPGRFESFFLSSTQAQQSYLQGFSKNILEASYDTKFEEINKVLFGREEGQQQGEERLQESVIVEISKKQIRELSKHAKSSSRKTISSEDKPFNLRSRDPIYSNKLGKLFEITPEKNPQLRDLDVFLSVVDMNEGALFLPHFNSKAIVVLVINEGEANIELVGIKEQQQRQQQEEQPLEVRKYRAELSEQDIFVIPAGYPVVVNATSDLNFFAFGINAENNQRNFLAGSKDNVISQIPSQVQELAFPGSAKDIENLIKSQSESYFVDAQPQKEEGNKGRKGPLSSILRAFY

SEQ ID NO: 2 - protein, 27 aa, Glycine max
MMRARFPLLLLGVVFLASVSVSFGIAY

SEQ ID NO: 3 - protein, 622 aa, Glycine max
MMRARFPLLLLGVVFLASVSVSFGIAYWEKQNPSHNKCLRSCNSEKDSYRNQACHARCNLLKVEEEEECEEGQIPRPRPQHPERERQQHGEKEEDEGEQPRPFPFPRPRQPHQEEEHEQKEEHEWHRKEEKHGGKGSEEEQDEREHPRPHQPHQKEEEKHEWQHKQEKHQGKESEEEEEEDQDEDEEQDKESQESEGSESQREPRRHKNKNPFHFNSKRFQTLFKNQYGHVCVLQRFNKRSQQLQNLRDYRILEFNSKPNTLLLPHHADADYLIVILNGTAILTLVNNDDRDSYNLQSGDALRVPAGTTYYVVNPDNDENLRMITLAIPVNKPGRFESFFLSSTQAQQSYLQGFSKNILEASYDTKFEEINKVLFGREEGQQQGEERLQESVIVEISKKQIRELSKHAKSSSRKTISSEDKPFNLRSRDPIYSNKLGKLFEITPEKNPQLRDLDVFLSVVDMNEGALFLPHFNSKAIVVLVINEGEANIELVGIKEQQQRQQQEEQPLEVRKYRAELSEQDIFVIPAGYPVVVNATSDLNFFAFGINAENNQRNFLAGSKDNVISQIPSQVQELAFPGSAKDIENLIKSQSESYFVDAQPQQKEEGNKGRKGPLSSILRAFY

SEQ ID NO: 4 - protein, 246 aa, Glycine max
MMRARFPLLLLGVVFLASVSVSFGIAYWEKQNPSHNKCLRSCNSEKDSYRNQACHARCNLLKVEEEEECEEGQIPRPRPQHPERERQQHGEKEEDEGEQPRPFPFPRPRQPHQEEEHEQKEEHEWHRKEEKHGGKGSEEEQDEREHPRPHQPHQKEEEKHEWQHKQEKHQGKESEEEEEDQDEDEEQDKESQESEGSESQREPRRHKNKNPFHFNSKRFQTLFKNQYGHVRVLQRFNKRSQQLQNL

SEQ ID NO: 5 - protein, 621 aa, Glycine max
MMRARFPLLLLGVVFLASVSVSFGIAYWEKQNPSHNKCLRSCNSEKDSYRNQACHARCNLLKVEEEEECEEGQIPRPRPQHPERERQQHGEKEEDEGEQPRPFPFPRPRQPHQEEEHEQKEEHEWHRKEEKHGGKGSEEEQDEREHPRPHQPHQKEEEKHEWQHKQEKHQGKESEEEEEDQDEDEEQDKESQESEGSESQREPRRHKNKNPFHFNSKRFQTLFKNQYGHVRVLQRFNKRSQQLQNLRDYRILEFNSKPNTLLLPHHADADYLIVILNGTAILTLVNNDDRDSYNLQSGDALRVPAGTTYYVVNPDNDENLRMITLAIPVNKPGRFESFFLSSTQAQQSYLQGFSKNILEASYDTKFEEINKVLFGREEGQQQGEERLQESVIVEISKKQIRELSKHAKSSSRKTISSEDKPFNLRSRDPIYSNKLGKLFEITPEKNPQLRDLDVFLSVVDMNEGALFLPHFNSKAIVVLVINEGEANIELVGIKEQQQRQQQEEQPLEVQKYRAELSEQDIFVIPAGYPVVVNATSDLNFFAFGINAENNQRNFLAGSKDNVISQIPSQVQELAFPGSAKDIENLIKSQSESYFVDAQPQQKEEGNKGRKGPLSSILRAFY

SEQ ID NO: 6 - protein, 505 aa, Glycine max
MAMRTKLSLAIFFFFLLALFSNLAFGKCKETEVEEEDPELVTCKHQCQQPQQYTEGDKRVCLQRCDRYHRMKQEREKQIQEETREKKEEESREREEEQQEQHEEQDENPYIFEEDKDFETRVETEGGRIRVLKKFTEKSKLLQGIENFRLAILEARAHTFVSPRHFDSEVVFFNIKGRAVLGLVSESETEKITLEPGDMIHIPAGTPLYIVNRDENDKLFLAMLHIPVSVSTPGKFEEFFGPGGRDPESVLSAFNWNVLQAALQTPKGKLEKLFDQQNEGSIFAISREQVRALAPTKKSSWWPFGGESKPQFNIFSKRPTISNGYGRLTEVGPDDDEKSWLQRLNLMLTFTNITQRSMSTIHYNSHATKIALVIDGRGHLQISCPHMSSRSSHSKHDKSSPSYHRISSDLKPGMVFVVPPGHPFVTIASNKENLLMICFEVNARDNKKFTFAGKDNIVSSLDNVAKELAFNNYPSEMVNGVFDRKESFFFPFELPREERGRRADA

SEQ ID NO: 7 - protein, 505 aa, Glycine max
MAMRTKLSLAIFFFFLLALFSNLAFGKCKETEVEEEDPELVTCKHQCQQPQQYTEGDKRVCLQRCDRYHRMKQEREKQIQEETREKKEEESREREEEQQEQHEEQDENPYIFEEDKDFETRVETEGGRIRVLKKFTEKSKLLQGIENFRLAILEARAHTFVSPRHFDSEVVFFNIKGRAVLGLVSESETEKITLEPGDMIHIPAGTPLYIVNRDENDKLFLAMLHIPVSVSTPGKFEEFFGPGGRDPESVLSAFSWNVLQAALQTPKGKLEKLFDQQNEGSIFAISKEQVRALAPTKKSSWWPFGGESKPQFNIFSKRPTISNGYGRLTEVGPDDDEKSWLQRLNLMLTFTNITQRSMSTIHYNSHATKIALVIDGRGHLQISCPHMSSRSSHSKHDKSSPSYHRISSDLKPGMVFVVPPGHPFVTIASNKENLLMICFEVNARDNKKFTFAGKDNIVSSLDNVAKELAFNNYPSEMVNGVFDRKESFFFPFELPREERGRRADA

SEQ ID NO: 8 - protein, 507 aa, Glycine max
MDHLKPRVSLLFLTDLTMATRAKLSLAIFLFFLLALISNLALGKLKETEVEEDPELVTCKHQCQQQRQYTESDKRTCLQQCDSMKQEREKQVEEETREKEEEHQEQHEEEEDENPYVFEEDKDFSTRVETEGGSIRVLKKFTEKSKLLQGIENFRLAILEARAHTFVSPRHFDSEVVLFNIKGRAVLGLVRESETEKITLEPGDMIHIPAGTPLYIVNRDENEKLLLAMLHIPVSTPGKFEEFFGPGGRDPESVLSAFSWNVLQAALQTPKGKLERLFNQQNEGSIFKISRERVRALAPTKKSSWWPFGGESKAQFNIFSKRPTFSNGYGRLTEVGPDDEKSWLQRLNLMLTFTNITQRSMSTIHYNLHATKIALVMDGRGHLQISCPHMSSRRSDSKHDKSSPSYHRISADLKPGMVFVVPPGHPFVTIASNKENLLIICFEVNVRDNKKFTFAGKDNIVSSLDNVAKELAFNYPSEMVNGVFERKESLFFPFELPSEERGRRAVA

SEQ ID NO: 9 - protein, 507 aa, Glycine max
MDHLKPRVSLLFLTDLTMATRAKLSLAIFLFFLLALISNLALGKLKETEVEEDPELVTCKHQCQQQRQYTESDKRTCLQQCDSMKQEREKQVEEETREKEEEHQEQHEEEEDENPYVFEEDKDFSTRVETEGGSIRVLKKFTEKSKLLQGIENFRLAILEARAHTFVSPRHFDSEVVLFNIKGRAVLGLVRESETEKITLEPGDMIHIPAGTPLYIVNRDENEKFLLAMLHIPVSTPGKFEEFFGPGGRDPESVLSAFSWNVLQAALQTPKGKLERLFNQQNEGSIFKISRERVRALAPTKKSSWWPFGGESKAQFNIFSKRPTFSNGYGRLTEVGPDDEKSWLQRLNLMLTFTNITQRSMSTIHYNSHATKIALVMDGRGHLQISCPHMSSRRSDSKHDKSSPSYHRISADLKPGMVFVVPPGHPFVTIASNKENLLIICFEVNVRDNKKFTFAGKDNIVSSLDNVAKELAFNYPSEMVNGVFERKESLFFPFELPSEERGRRAVA

SEQ ID NO: 10 - protein, 507 aa, Glycine max
MDHLKPRVSLLFLTDLTMATRAKLSLAIFLFFLLALISNLALGKLKETEVEEDPELVTCKHQCQQQRQYTESDKRTCLQQCDSMKQEREKQVEEETREKEEEHQEQHEEEEDENPYVFEEDKDFSTRVETEGGSIRVLKKFTEKSKLLQGIENFRLAILEARAHTFVSPRHFDSEVVLFNIKGRAVLGLVRESETEKITLEPGDMIHIPAGTPLYIVNRDENEKLLLAMLHIPVSTPGKFEEFFGPGGRDPESVLSAFSWNVLQAALQTPKGKLERLFNQQNEGSIFKISRERVRALAPTKKSSWWPFGGESKAQFNIFSKRPTFSNGYGRLTEVGPDDEKSWLQRLNLMLTFTNITQRSMSTIHYNSHATKIALVMDGRGHLQISCPHMSSRRSDSKHDKSSPSYHRISADLKPGMVFVVPPGHPFVTIASNKENLLIICFEVNVRDNKKFTFAGKDNIVSSLDNVAKELAFNYPSEMVNGVFERKESLFFPFELPSEERGRRAVA

SEQ ID NO: 11 - protein, 598 aa, Glycine max
MIARFPLLPLLLLGIVFLASVSVSLQVKPPSHHECLQSYESETDPYKRKACKLSCKFVPEHGEQQHEEEEREREHPQPHPPHEERGRRERGRQEGEKEEKEQDERELQPRPFPRPEEHEGEQWKGRHRHEDPEERARMRLREAERIEREREEQRVREEEEKLRRREKKEQREEKEEEGQGSEDSHSKRQNNPFHFSSNRFQTLFKNQHGHLRVLQRFDQRSPQLENLRDYRVVELMAKPNTLFLPHHADADFLLLVLSGRALINLVKPDDRDPYYLDRGYAQRIPAGTTVYLVNPDKKKDLRVIKLAIPVNKPGNFEDFFLSSTQDQQSYLQGFSENILEASFNTKFEEINRVLFGGEGRRHQQEGVILELSKEQIRELSKRAKSSSRSTNSFDYEPFYLRGSQISSNNFGKFYEITPEKNPQLRDFDILLNTVDINELVILIIETKQIQGGLLLPHYNSKAIVILMVTEGEANIELVGLKEQQQGEETREVRKYRAELSEDDIFVIPAAYPFVVNATSNLNFVAFGINAENNQRNFLAGSKDNVIRQIQKQVKELAFPAPAGSAQDIENLIKNQRESYFADAQPLQKEEGKKGSFVF

SEQ ID NO: 12 - protein, 598 aa, Glycine max
MIARFPLLPLLLLGIVFLASVSVSLQVKPPSHHECLQSCESETDPYKRKACKLSCKFVPEHGEQQHEEEEREREHPQPHPPHEERGRRERGRQEGEKEEKEQDERELQPRPFPRPEEHEGEQWKGRHRHEDPEERARMRLREAERIEREREEQRVREEEEKLRRREKKEQREEKEEEGQGSEDSHSKRQNNPFHFSSNRFQTLFKNQHGHLRVLQRFDQRSPQLENLRDYRVVELMAKPNTLFLPHHADADFLLLVLSGRALINLVKPDDRDPYYLDRGYAQRIPAGTTVYLVNPNKKKDLRVIKLAIPVNKPGNFEDFFLSSTQDQQSYLQGFSENILEASFNTKFEEINRVLFGGEGRRHQQEGVILELSKEQIRELSKRAKSSSRSTNSFDYEPFYLRGSQISSNNFGKFYEITPEKNPQLRDFDILLNTVDINELVILIIETKQIQGGLLLPHYNSKAIVILMVTEGEANIELVGLKEQQQGEETREVRKYRAELSEDDIFVIPAAYPFVVNATSNLNFVAFGINAENNQRNFLAGSKDNVIRQIQKQVKELAFPAPAGSAQDIENLIKNQRESYFADAQPLQKEEGKKGSFVF

SEQ ID NO: 13 - protein, 598 aa, Glycine max
MIARFPLLPLLLLGIVFLASVSVSLQVKPPSHHECLQSCESETDPYKRKACKLSCKFVPEHGEQQHEEEEREREHPQPHPPHEERGRRERGRQEGEKEEKEQDERELQPRPFPRPEEHEGEQWKGRHRHEDPEERARMRLREAERIEREREEQRVREEEEKLRRREKKEQREEKEEEGQGSEDSHSKRQNNPFHFSSNRFQTLFKNQHGHLRVLQRFDQRSPQLENLRDYRVVELMAKPNTLFLPHHADADFLLLVLSGRALINLVKPDDRDPYYLDRGYAQRIPAGTTVYLVNPDKKKDLRVIKLAIPVNKPGNFEDFFLSSTQDQQSYLQGFSENILEASFNTKFEEINRVLFGGEGRRHQQEGVILELSKEQIRELSKRAKSSSRSTNSFDYEPFYLRGSQISSNNFGKFYEITPEKNPQLRDFDILLNTVDINELVILIIETKQIQGGLLLPHYNSKAIVILMVTEGEANIELVGLKEQQQGEETREVRKYRAELSEDDIFVIPAAYPFVVNATSNLNFVAFGINAENNQRNFLAGSKDNVIRQIQKQVKELAFPAPAGSAQDIENLIKNQRESYFADAQPLQKEEGKKGSFVF

SEQ ID NO: 14 - protein, 598 aa, Glycine max; identical to SEQ ID NO: 13

SEQ ID NO: 15 - protein, 633 aa, Glycine max
MMRARFPLLLLGVVFLASVSVSFGIAYWEKQNPSHNKCLRSCNSEKDSYRNQACHARCNLLKVEEEEECEEGQIPRPRPQHPERERQQHGEKEEDEGEQPRPFPFPRPRQPHQEEEHEQKEEHEWHRKEEKHGGKGSEEEQDEREHPRPHQPHQKEEEKHEWQHKQEKHQGKESEEEEEDQDEDEEQDKESQESEGSESQREPRRHKNKNPFHFNSKRFQTLFKNQYGHVRVLQRFNKRSQQLQNLRDYRILEFNSKPNTLLLPHHADADYLIVILNGTAILTLVNNDDRDSYNLQSGDALRVPAGTTYYVVNPDNDENLRMITLAIPVNKPGRFESFFLSSTQAQQSYLQGFSKNILEASYDTKFEEINKVLFGREEGQQQGEERLQESVIVEISKKQIRELSKHAKSSSRKTISSEDKPFNLRSRDPIYSNKLGKLFEITPEKNPQLRDFDILLNTVDINELVILIIETKQIQGGLLLPHYNSKAIVILMVTEGEANIELVGLKEQQQRQQQEEQPLEVRKYRAELSEQDIFVIPAGYPVVVNATSDLNFFAFGINAENNQRNFLAGSKDNVISQIPSQVQELAFPGSAKDIENLIKSQSESYFVDAQPQQKEEGNKGRKGPLSSILRAFY

SEQ ID NO: 16 - protein, 505 aa, Glycine max
MAMRTKLSLAIFFFFLLALFSNLAFGKCKETEVEEEDPELVTCKHQCQQPQQYTEGDKRVCLQRCDRYHRMKQEREKQIQEETREKKEEESREREEEQQEQHEEQDENPYIFEEDKDFETRVETEGGRIRVLKKFTEKSKLLQGIENFRLAILEAHAHTFVSPRHFDSEVVFFNIKGRAVLGLVSESETEKITLEPGDMIHIPAGTPLYIVNRDENDKLFLAMLHIPVSVSTPGKFEEFFGPGGRDPESVLSAFSWNVLQAALQTPKGKLEKLFDQQNEGSIFAISREQVRALAPTKKSSWWPFGGESKPQFNIFSKRPTISNGYGRLTEVGPDDDEKSWLQRLNLMLTFTNITQRSMSTIHYNSHATKIALVIDGRGHLQISCPHMSSRSSHSKHDKSSPSYHRISSDLKPGMVFVVPPGHPFVTIASNKENLLMICFEVNARDNKKFTFAGKDNIVSSLDNVAKELAFNNYPSEMVNGVFDRKESFFFPFELPREERGRRADA

SEQ ID NO: 17 - protein, 506 aa, Glycine max
MDHLKPRVSLLFLTDLTMATRAKLSLAIFLFFLLALISNLALGKLKETEVEEDPELVTCKHQCQQQRQYTESDKRTCLQQCDSMKQEREKQVEEETREKEEEHQEQHEEEEDENPYVFEEDKDFSTRVETEGGSIRVLKKFTEKSKLLQGIENFRLAILEARAHTFVSPRHFDSEVVLFNIKGRAVLGLVRESETEKITLEPGDMIHIPAGTPLYIVNRDENEKLLLAMLHIPVSTPGKFEEFFGPGGRDPESVLSAFSWNVLQAALQTPKGKLERLFNQQNEGSIFKISRERVRALAPTKKSSWWPFGGESKAQFNIFSKRPTFSNGYGRLTEVGPDDEKSWLQRLNLMLTFTNITQRSMSTIHYNSHATKIALVMDGRGHLQISCPHMSSRSDSKHDKSSPSYHRISADLKPGMVFVVPPGHPFVTIASNKENLLIICFEVNVRDNKKFTFAGKDNIVSSLDNVAKELAFNYPSEMVNGVFERKESLFFPFELPSEERGRRAVA

SEQ ID NO: 18 - protein, 596 aa, Glycine max
MIARFPLLPLLLLGIVFLASVSVSLQVKPPSHHECLQSCESETDPYKRKACKLSCKFVPEHGEQQHEEEEREREHPQPHPPHEERGRRERGRQEGEKEEKEQDERELQPRPFPRPEEHEGEQWKGRHRHEDPEERARMRLREAERIEREREEQRVREEEEKLRRREKKEQREEKEEEGQGSEDSHSKRQNNPFHFSSNRFQTLFKNQHGHLRVLQRFDQRSPQLENLRDYRVVELMAKPNTLFLPHHADADFLLLVLSGRALINLVKPDDRDPYYLDRGYAQRIPAGTTVYLVNPDKKKDLRVIKLAIPVNKPGNFEDFFLSSTQDQQSYLQGFSENILEASFNTKFEEINRVLFGGEGRRHQQEGVILELSKEQIRELSKRAKSSSRSTNSFDYEPFYLRGSQISSNNFGKFYEITPEKNPQLRDFDILLNTVDINELVILIIETKQIQGGLLLPHYNSKAIVILMVTEGEANIELVGLKEQQQGEETREVRKYRAELSEDDIFVIPAAYPFVVNATSNLNFVAFGINAENNQRNFLAGSKDNVIRQIQKQVKELAFPAGSAQDIENLIKNQRESYFADAQPLQKEEGKKGSFVF

SEQ ID NO: 19 - protein, 438 aa, Glycine max
MMRVRFPLLVLLGTVFLASVCVSLKVREDENNPFYLRSSNSFQTLFENQNGRIRLLQRFNKRSPQLENLRDYRIVQFQSKPNTILLPHHADADFLLFVLSGRAILTLVNNDDRDSYNLHPGDAQRIPAGTTYYLVNPHDHQNLKIIKLAIPVNKPGRYDDFFLSSTQAQQSYLQGFSHNILETSFHSEFEEINRVLLGEEEEQRQQEGVIVELSKEQIRQLSRRAKSSSRKTISSEDEPFNLRSRNPIYSNNFGKFFEITPEKNPQLRDLDIFLSSVDINEGALLLPHFNSKAIVILVINEGDANIELGIKEQQQKQKQEEEPLEVQRYRAELSEDDVFVIPAAYPFVVNATSNLNFLAFGINAENNQRNFLAGEKDNVVRQIERQVQELAFPGSAQDVERLLKKQRESYFVDAQPQQKEEGSKGRKGPFPSILGALY

SEQ ID NO: 20 - protein, 439 aa, Glycine max
MMRVRFPLLVLLGTVFLASVCVSLKVREDENNPFYLRSSNSFQTLFENQNGRIRLLQRFNKRSPGLENLRDYRIVQFQSKPNTILLPHHADADFLLFVLSGRAILTLVNNDDRDSYNLHPGDAQRIPAGTTYYLVNPHDHQNLKIIKLAIPVNKPGRYDDFFLSSTQAQQSYLQGFSHNILETSFHSEFEEINRVLFGEEEEQRQQEGVIVELSKEQIRQLSRRAKSSSRKTISSEDEPFNLRSRNPIYSNNFGKFFEITPEKNPQLRDLDIFLSSVDINEGALLLPHFNSKAIVILVINEGDANIELVGIKEQQQKQKQEEEPLEVQRYRAELSEDDVFVIPAAYPFVVNATSNLNFLAFGINAENNQRNFLAGEKDNVVRQIERQVQELAFPGSAQDVERLLKKQRESYFVDAQPQQKEEGSKGRKGPFPSILGALY

SEQ ID NO: 21 - protein, 605 aa, Glycine max
MMRARFPLLLLGLVFLASVSVSFGIAYWEKENPKHNKCLQSCNSERDSYRNQACHARCNLLKVEKEECEEGEIPRPRPRPQHPEREPQQPGEKEEDEDEQPRPIPFPRPQPRQEEEHEQREEQEWPRKEEKRGEKGSEEEDEDEDEEQDERQFPFPRPPHQKEERKQEEDEDEEQQRESEESEDSELRRHKNKNPFLFGSNRFETLFKNQYGRIRVLQRFNQRSPGLQNLRDYRILEFNSKPNTLLLPNHADADYLIVILNGTAILSLVNNDDRDSYRLQSGDALRVPSGTTYYVVNPDNNENLRLITLAIPVNKPGRFESFFLSSTEAQQSYLQGFSRNILEASYDTKFEEINKVLFSREEGQQQGEQRLQESVIVEISKEQIRALSKRAKSSSRKTISSEDKPFNLRSRDPIYSNKLGKFFEITPEKNPQLRDLDIFLSIVDMNEGALLLPHFNSKAIVILVINEGDANIELVGLKEQQQEQQQEEQPLEVRKYRAELSEQDIFVIPAGYPVVVNATSNLNFFAIGINAENNQRNFLAGSQDNVISQIPSQVQELAFPGSAQAVEKLLKNQRESYFVDAQPKKKEEGNKGRKGPLSSILRAFY

SEQ ID NO: 22 - protein, 604 aa, Glycine max
MMRARFPLLLLGLVFLASVSVSFGIAYWEKENPKHNKCLQSCNSERDSYRNQACHARCNLLKVEKEECEEGEIPRPRPRPQHPEREPQQPGEKEEDEDEQPRPIPFPRPQPRQEEEHEQREEQEWPRKEEKRGEKGSEEEDEDEDEEQDERQFPFPRPPHQKEERKQEEDEDEEQQRESEESEDSELRRHKNKNPFLFGSNRFETLFKNQYGRIRVLQRFNQRSPQLQNLRDYFILENSKPNTLLLPNHADADYLIVILNGTAILSLVNNDDRDSYRLQSGDALRVPSGTTYYVVNPDNNENLRLITLAIPVNKPGRFESFFLSSTEAQQSYLQGFSRNILEASYDTKFEEINKVLFSREEGQQQGEQRLQESVIVEISKEQIRALSKRAKSSSRKTISSEDKPFNLRSRDPIYSNKLGKFFEITPEKNPQLRDLDIFLSIVDMNEGALLLPHFNSKAIVILVINEGDANIELVGLKEQQQEQQQEEQPLEVRKYRAELSEQDIFVIPAGYPVVVNATSNLNFFAIGINAENNQRNFLAGSQDNVISQIPSQVQELAFPGSAQAVEKLLKNQRESYFVDAQPKKKEEGNKGRKGPLSSILRAFY

SEQ ID NO: 23 - protein, 483 aa, Glycine max
MGSRMSLLMLFLVLCHGVALTMGLWRTEVEEREEGHPRPDKLFMMLKSKRVAQTDAGEMRVLESYGGRILERRLNIGEINMEPRSLFIPQYIDSTLIIFIRSGEAKLGFIYKDKLAFRHLKMGDVYQIPAGSAFYLVNIEEAQKLHIICSIDPSESLGIDIEQSFYLGGGAHPASVLSGFEPQILETAFNASGEELRKMFTRQHEGPIVHVGDSHATSIWTKFLQLKEEDKLQHLREMVQQEQEEEEEEFVAEEEKQTSWSWRKLLESVFGDEIKNTREKVTKKSPHSCNLYDRKPDFKNSYGWSVALDGSEYSPLKSSGVGIYHVNLSAGSMMAPHVNPRATEYGIVLKGSGRIQIVFPNGSNAMDAHIKEGDVFFIPRYFAFCQIASRGEPLEFFGFTTSAQKNRPQFLVGATSLMRTMVGPELAAAFGVSEETMNRVARAQHEAVILPTPWAAHAHDHKVKEEVVVDIFQKLARNEIMGF

SEQ ID NO: 24 - protein, 383 aa, Glycine max
MLNMKKRKYAKENLKKFQIEECKSVSTPMNQKEKFSKKEGRALINLVKPDDRDPYYLDRGNYAQRTLAGTTVYLVNPDKKNDLRIITLAIPNFFLSSTQDQQSYLQRFSENILEASFNSEFEEINRVLFGGEERQQQCEQRQQQEGVIVEISKEQIRQLSRRAKSSSRKTISSEDEPFNLRSHNPIYSNNFGKFFEITPEKNPQLGTWISCSVLWISTKLLNNDLLFLCSQILGALLLPHFNSKAIVILVIDEGEASIEFVGIREQQQEEEIREVRKYRAELSEDDIFVIPAAYPFVVNATSNLNFLAFGVNAENNQRNFLAGDKDNVVSQIQRQVKELAFPAGSARDVEKLIKNKKKSYFADAQPQQKKGGRKGRKGPLSTE

SEQ ID NO: 25 - protein, 296 aa, Glycine max
MHPAGIRRRVAKNLHWATRGPNCVRGGSHATTIWTKFLQLKEEDKLYHLREIVQQEEGKEEEEFVDEEEQQTSWSWRKILESVFEDEVKNTREKVTKTSPLSCNLYDRKPDFKNCYGWSVDQDGSEYSPLKSSGVGIYHVNLSAVNMMAPHVNPRAKEYGIGLKGSGRIQIVFPNGSNAIYMDAHIKEGDVFFIPRYFAFCQIASKNEPLEFFGFTTSAQKNRPQFLVGATSLMRTMVGPELAAAFGVSEETMRRMARAQHEAVILPTPWTAHAHDHKVVVDRVQKLTKNEMVMGF

SEQ ID NO: 26 - protein, 502 aa, Phaseolus vulgaris
MKINLSLPILLFFLLALFSNLSLAKKETKVVGDPELKTCKHQCLQQQKYSEDDKLICLRSCDEYHGLKHEREKQIEEQIHEHEGSSEEHEEEEESSEKKEEQVEEGGEEEEEYPYIFEEDRDFDTKFETEDGRIRLLKKFTEKSELLKGIENIRLTIVEAKPHTFVAPRHFDSDVVLFNIKGRAVLGWVKESETEKFILESGDMLAIPAGTPLYIVNRDENEKLFLAMLHVPVSTPGKFQEFFGPGGRDPESFLSAFGWNVLQAALQSPKGQLEKLFNQQNGGSIFEISRKKVQEMAPKKTSWWPFGGPSTAEFNLFTKPPTFSNRHGRLTEVGPPDSRSLLEKLNIMLSFTNITMKSMSTIHYNSHATKIALVIDGKGHLQIVCPHISSRSNSKHEKSSPSNHRISADLKPGMLFVVPPGHPFVTFSSRKENLQIISFEINARDNKKFTFAGKDNIVSSMDDLAKELAFNYPSEIVNKIFDRKESFFFPFELPQSDPRADA

SEQ ID NO: 27 - protein, 511 aa, Phaseolus angularis
MAMKINLSLPIFLFFLLAFFSTLASAKRETEVAADPELKTCKHQCLQQLQYTEADKQICLQRCDMYIDLKHEHEKQLEEKTRKKKKHEGAPREHDEEEEGGEKEEEEEAEEGGEEEEEYPYIFEEDTDFDTKVETDDGRIRVLKKFTEKSKLLKGIENIRLALLEAGAHTFVAPRHFDSDVVLFNIKGRAVLGLVKESETEKFVLESGDMLPIPAGTQVYIVNRDENEKLFLAMLHIPVSTPGKFEEFFGPGGRDPESVLSAFGWNVLQAALQSPKGELEKLFDQQNGGSIFQISREQVQELAPKKTYWWPFRGHSTAEFNLFTMSPTFSNRNGILTEAGPDSSSLLQKHNIMLSFTNITKESMSTILYNSHATKIAVVIGGKGGLQIVCPHITSSSNGNHEKSSPSYHRISAELKPGMVFVVPPGHPFVTFSSRKENLQILSFEINAQDNKKFTFAGKDNIVSSMDDVAKELGFNRPSKIVNRIFDRKESFFFPFKLPQQEQGHDRRAYA

SEQ ID NO: 28 - protein, 587 aa, Phaseolus angularis
MRSQFPLFLVLGIVFLASVSVSLTEKPSWSKCVQTCESEKDASRVQKCQLRCKHVSERGEKEEEEERGIPEPHPSHEKKEEEKEELPPPHSREHEREEEQWKGRQRREDPEERERVRQREREKEEVMREREKEEEMREREKEVEMREREREKEREKGEKYRRREKKEKWEEVEEDPGFPNSDSQTQNNPFHFSSNRFHTLFKNQHGHIRVLQSFDQRSSTQLQNLRDYRVVEFKSRPHTLLLPHHANADFLLVVLSGRALINLVEPDNRDPYYLDPGYAQIIPAGTTFYLVNPERNKNLRVIKLAIPVNKPGKFENFFLSSTQEQQSYLQGFSEDILEASFDSKFEDISKVLFGEERLRQRQQEGVIVELSNEKIRELSRKAESSSMKINSFEYKPFDLRSSHPIYSSKLGAFFEITPEKNPHLQELNIFLNYVDIKKGGLLLPHYNSKAIVILVVNEGEANIELVGLTEQQQQKRQEVQRYTAELSKDDVFVIPAAYPVAINATSNLNFFAFGINAENNQRNFLAGEKDNVISEIPRQVFEVAFPGSGEDVEKLIKKKRESYFVAAQPQQKEENHIPLSSILGALH

SEQ ID NO: 29 - protein, 481 aa, Cajanus cajan
MAMRTNLSLAIFLFFSFALFSNLALCKKVTEVEDPELITCKHQCQQQCEYTEDDKRACVQRCDVYHRTKQEREKQIQEETREKKEREMEENPYVFEESSFDTKVETESGRIRLLQKFTKKSKLLQGIDNFRLAIIEAKARAFMSPLHFDSEVVLFIVKGQALLGMVRESETEKFSLEAGDMLRIPAGTPLYIANRDDNEKLFFAMLHMPISTPGKFEEFFGPGGQDPESVLSAFGWNVLQAALQTPKGKLEKLFNQQNEGRIFEISREQLQALAPKKGSWWPFGGPSKAPFNLFRKYPVFSNRNGRLTEVGPSDSKSGLEALNIMLTFTNITQRSMSTIHYNSHATKIALVINGTGHLQIACPHLASKSESEHEKSSPSYHSISSNLKPGTVFVVPPGHPFVTMASRKENLLIICFEINARENKKFTFAGKNNIVSSLDTVAKELAFNYPADMVNQVFDRKEEFFFPFVLPREERGRYADA

SEQ ID NO: 30 - protein, 557 aa, Cajanus cajan
MMRARFPLLLLLGLVFLASVSVSFGIAYWERDNLVHSKCLRSCNAENDAYRHEACHARCNLLKHDGAEAHQSHEETGHRERARQEGEKEEKKHHEEEGHPHPFPRPHQQKEHEHEHEQEGQSRPFPRRHQPHQEHQHQHEPEEEEGQGSESESQTHHNPFHFDSNMFHTFFKNQYGHIRVLPRFDQRSSKLQNLRDYRVVEFQSKPNTLLLPNHADAEFILLVLNGRAILNIVSPDDRESYDLHQGDAQRIPAGTTYYLINPDDKETLKVATLAMPVNEHANFENFYLSSTESQKSYLQAFSKNILEASFDTEFEEINEVLFGEEKHKHGEQSQQEGVIVKLSKEQIRELSKHAKSSSRKTISSEDKPFNLRSREPIYCNKFGKFFEITPEKNPQLRELDLFLSFVELNEGALFLPHYNSKAIVILVVNEGEANIELVGISEQHPRRFRAELSENDIFVIPAAFPVVLNATSNFNFYAFGVNAENNQRNFLAGEKDNVIMQIQRQVKELAFPGSAQDVEKLLKNQKDSFFVDAQPQQNDEGNKGRKGPLSSILGAFY

SEQ ID NO: 31 - protein, 508 aa, Vigna radiata
MAIKINLSLPISLFFLLALFSTLAFAKKETEVAADPELKTCKHQCLQQLQYTEAEKQMCLQSCDMYHGLKHEYEKQIEEKIRKKKKHEGTPQEHDEEEESGEKEEEVEEGGEEEEEYPYIFEEDWDFDTKVETDDGRIRVLKKFTEKSKLLKGIENIRLAILEARAHAFVAPRHFDSDVVLFNIKGRAVLGWVKESETEKFVLESGDMLPIPAGTQVYIVNRDENEKLFLAMLHIPISTPGKFEEFFGPGGRDPESVLSAFGWNVLQAALQSPKGELEKLFDQQNGGSIFQISREQVQELAPKKTYWWPFSGPSTAEFNLFTMSPTFSNRYGTLTEAAPSDSRSLLQKHNIMLSFTNITKESMSTILYNSHATKIAVVISGKGGLQIVCPHITSRSHGKHEKSSPSYHRISAELKPGMVFVVPPGHPFVTFSSRKENLQILSFEINAQDNKKFTFAGKDNIVSSMDDLAKELGFNHPSKIVNRIFDRKESLFFPFKLPHEGRDGRADA

SEQ ID NO: 32 - protein, 595 aa, Vigna radiata
MRSRFPLFLVLGIVFLASVSVSLTEKPSRSKCVQTCESEKDAARVEKCQLRCKHVSERGEKEEEEEKVIPEPHPSHEEKEEHEREEEQWKGRQRREDPEERERVRQRERKKEEEMREREKEEEMREREKEGEREKEEEMREREKEEEMREREKEEEMRERERKKGEKYRRREKKEKWEEVEEDRGYSDSQTQNNPFHFSSNRFHTLFKNQHGHMRVLQSFDQRSSIQLQNLRDYRVVEFKSRPHTLLLPHHANADFLLVVLSGRALINLVEPDNRDPYYLDPGYAQIIPAGTTFYLVNPERNKNLRVIKLAIPVNKPGKFENFFLSSTQEQQSYLQGFSEDILEASFDSKFEDISKVLFGEEILRQRQQEGVIVELSNEKIRELSRSAESSSRKINSFKYKPFDLRSSHPIYSNKLGAFYETTPEKNPHLQELNIFLNYVDIKKGGLLLPHYNSKAIVILVVNEGETNIELVAVTEQQQQQKRQEVQRYTAELSKDDVFVIPAAYPVAINATSNLFNFFAFGINAENNQRNFLAGEKDNVISEIPRQVFEVAFPGSGEDVEKLIKKKRESYFVAAQPQQKEDNHIPLSSILGALH

SEQ ID NO: 33 - protein, 493 aa, Medicago truncatula
MAMKTKLSLAIFLFFLLALLCSNLAIAIGREETDPELKTCIHQCKQQRQYDEEDKGICMDKCEDYHRMKQEREKRQHQHQREHEHQHEREHEHEEDENPYVFEDRDFETKIDTDDGRVMALNMFDQKSKLLRNFENYGLTILEAKGHAFVSPHHFDSEVIFFNVKGRGIIGLVMEDKTERFNLEAGDIMRVPAGTPMYLVNRDENEKLFVAALHMPSSASAPVNLEAFFGPAGRDPESVLTAFSSKVLQAAFKSPKGKLESFLDEQNKGRIFKIQKEDLSGLAPKKSIWPFGGQFKNPFNIFSNNPAFSNQFGSLFEVGPSEITSGLDGLNLMLTFANITKGSMSTIYYNTNANKIALVIDGEGEFEMACPHMSSSSSHSKQRRSSSTSYQKINARLRPGTVFVVPAGHPFVTIASKNNNLKIVCFEVNAQRNKKLAFAGKNNIVSALDKTAKKLAFDKSAEKVDEIFKRDEEFFFPYDVEDESKEEHGRADE

SEQ ID NO: 34 - protein, 471 aa, Cicer arietinum
MAIKAKVSLAFFLFFLLSLLCSNLVISKEERDPEIKTCKHQCRQQLQYSEEDKNVCMEECDEYHRMKKEREKRKEQDQDEDENENPYVFKSKDFDTRIDTEDGRVLVLNKFNDKSKLLRNIENYGLAVLEVKGHAFVSPHHFDSEVIIFNVKGKAIIGLVMEDKTERFNLESGDIMRVPAGTPMYLVNRDENEKLFIAALHMPPLSASAPVNLEAFFEPGGRNPESVLTAFSSKVLQAALKTPKEKLERFFDYQRRERRIFKISKEDVHGLAPKRSIWPFGGQFKGPFNIFTNRPSFSNQYGSLFEVGPSEEKSGLDELNLMLTLANITKGCMSTIYYNTNANKIAFVTEGEGDFEMACPHLKEKKSSSTSYHKISGRLKAGMVFVVPAGHPFVTMASKKNNLVIVCFEVNAERNKKLAFAGKRNIVSALDKRAKEVAYEYPAEKVDEIFERDEEFFFPYEEGEEEERADA

SEQ ID NO: 35 - protein, 490 aa, Lupinus angustifolius
MKFITINLSLPIFLFILVATSCLGTPDEEAELDPELTTCIHQCKHQDKYSRDDKQECTRGCHKYHQMKKQRERQIEEQVRRRKEHEMEQVKEKQRHQGEEEEEEEEEENPFLFDDDDFETRFETEDGRVRVLHRFTKKSNLLRGIHNFRLATLEAEGHTFVSPCHFDSEAVFFVVKGRATIGMVREEKTERFNLEQGDIIRVPSGTPVYLVNRDHNQKLFIAKLHMPLAVPGKFEAFYGPGGRNPESFLTAFSWELLEAALKSPREKLERLFQKQEKGGIFKISKEDVESVSKVKGSGIWPFGGRSSGPFNIFKSPIMSNQYGRLFETNLDDHSQLKDLNLILSFANITNGSMTAPMYATRATNVIMVSDGKGYIEMVCPHVSSTHSYHPFKASLKPGVVFVVPTGHPFIIVASNKNNLQLLRFQLHAQGDNTLTFAGKKNVVKALDKEAKELAFNYPADKVDEIFSRDEDLFLPGPDSYDFEEHARAYA

SEQ ID NO: 36 - protein, 471 aa, Trifolium pratense
MAIKTKLSIFIFLFFLINIAICREETDPELKTCKHQCHQQRQYDEHDKRICMKECDEYHEMKQEREKHKEDKDDEKEVENPYVFEYKDFDTILDTEDGRISVLNMFDQKSKLLRNVENYGLAVLEAKAHAFVSPHHFDSEVIFFNMKGRGIIGLVEQDNTERFNLEAGDIIRVPAGTPIYLVNRDENEKLFIAALHMPSSPGSAPLNFEAFFGPAGRNPESVLTAFSSKVLQAAFKTSKGKLESVLEEQKKERIFKIRKEDVHGMAPKKSIWPFGGQFNGPFNIFSNKPSFSNQFGSLFEIGPSESKSGLEGLNVMLSFANITKGSMSTIYYNTNANKIAFVVDGEGYFEMACPHYMSSKHRKSSSAYHKMNARLRPGMVFVVPAGHPFVTIASKNNNLKIVCFEVNAERNKKLAFAGKKNIVVALDKTAKKLSFDSSPEKVDEIFKRDEEFFFSYDVDEESKEDRGHADV

SEQ ID NO: 37 - protein, 502 aa, Juglans regia
MAFKPKIPIALLLLTSLLAICAGLALAMQDPELKQCKHQCRHQRQFDEQEKEHCQRSCDEYHIEKKARERAERRRSEEGSSREEGYEEEELGGEREEENPYVFEDEDFETRVRTDEGRIQVLEKFTKRSKLLRGIENFRVAILEANPQTFISPAHFDAELVVFVAKGRATITTVREEKRENFNVEQGDIMRIPAGTPVYLINRDENEKLYIVKILRPVSVPGHFEAFHGSGGEDPESFYRAFSWEVLEAALKTPRDQLEKLFGKQTQGVIIKASKEQIRSMSKHEETTPRIWPFGGDSTHPFNLFHKRPSQSNQFGRLFETDPKECKQLQDLDLMVSFANITKGSMAGPYYNSRATKISVVIEGEGYFEMACPHLSSSGSRGQREGSGSSRRRSRSGPSYQQIRGRLRPGMVFVAPAGHPVAVIASRNKNLQVLCFDVNAQGNIRFPLAGKNNIVNEFEKEAKELAFNFPAREVEKIFRNQDQEFFFPGPSRQPEEGGRAFE

SEQ ID NO: 38 - protein, 591 aa, Macleaya cordata
MAFKTKLPLLFFLFSLILLSATLCLGYESSEDPQKEYHQCQRRCQQQERGQRQQCQQQCEQQRREREEKEEGRGNSHGRDPQKEYKQCQHRCEQQERGQRQQCQQQCEQQRREREEREEGRGNSHGRDPQKEYKQCQHRCEQQERGQRQQCQQQCEERRREQEREQEGRGGSGYTDWRRRSEEEQQQEQENNPYYYSDQHFRTRIRTSEGHVRLLQRFTEKSELLRGIDNYRVAILEANPNTFIIPNHQDADSLLYVVKGRGTISLMRQDNRETHNLERGDVMRIPAGAIVYLINRDNNEKLFIAKLLRPISVPGHVEAFFGPGGEDPESFYSSFSHEVLEAAFNTPMEKLQRLIGQQKRGAIIKASQEQIKALSPHGRWPFGSESNAPYNLLHHRPMHSNRYGQLYQVNPNHYKQLEDMDMAISFANISSGGMAGPYYNSRATKISVVLEGKGYFEMACPHLSSSSSAGGHQGGRGGSEQHHEQRSGRYQKVSARLSPGDVIVAPAGHPIVLVASRNQNLQIVCFEVNAHDNEKNLLAGRDSMLNQIEKEAKELAFNLPAREVEEIFNNQKETFFFPGPSHRQEQGRAFE

SEQ ID NO: 39 - protein, 592 aa, Erythranthe guttata
MRCFCLLFFALLLASATIALGYKEDPELKQCKHQCEAQKQFGEEKIRTCVQKCEDYVREKQRREHGGGSTTFFEEEDDDLNRRSPIERLRECTKGCDRSKQKERCQSRCQETYEKEKERYEGGGNPVDPEQEYQECRQECRRQSREGGSRRGTSQERCEERCQEQRREREREQPGRREGGGNMYEGREREEEEEERQQQKKQGRENNPYVFEERHFFTGMETQHGRLRILPKFTDRSKLLKGIENYRVAILEAEPQTFIVPNHWDADTLIFVANGRGAVSLMRQERRESFNIKQGDIFRINAGTTAYLINRDSNQKLVLAKLLQPVNTPGQFEAFFGAGGENPESFYTAFSNEILEAAFNTRRDRLQRLFGQQKQGVIIKASEEQVRAMSHHEEGGIWPFGGESKGTFNLYDQRPSHSNEYGQLFEVDSSQFRQLRDLDIAVSLANITQGAMIAPSYNSKSTKITVVVDGEGYFEMACPHISQQQQGAGQRGYEKVSSRLKRGTVVVVPAGHPFVAVASNNQNLQLLCFEVNAYNNEKFTLAGRRNVMNQLEREAKELAFGMPAREVDEIFRNQKEEFFFKGPRQQGGRSDE

SEQ ID NO: 40 - protein, 597 aa, Erythranthe guttata
MRCFCLLLFALLLASATIALGYKEDPELKQCKHQCEAQKQFGEEKIRTCIQQCEDYVREKQRREHGGEGGGSTTFFEEEDDDLNRRSPIERLRECTKGCDRSKQKERCQSRCQETYEKEKERYEGGGNPVDPEQEYQECRQECRRQSREGGSRQERCEERCQEQRREREREQPGRREGGGGMYMYEGREREEEEERRQQKQNPYVFEERHFFTGMETQHGRLRILPKFTDRSKLLKGIENYRVAILEAEPQTFIVPNHWDADTLIFVANGRGAVSLMRQERRESFNIKQGDIFRINAGTTAYLINRDSNQKLVLAKLLQPVNTPGQFEAFFGAGGENPESFYTAFSNDILEAAFNTRRDRLQRLFGQQKQGVIIKASEEQVRAMSHHEEGGIWPFGGESKGTFNLYDQRPSHSNEYGQLFEVDSSQFRQLRDLDIAISLANITQGAMIAPSYNSKSTKITVVVDGEGYFEMACPHISQSQSQGGRQQRQQQGAGQRGYEKVSSRLKRGTVVVVPAGHPFVAVASNNQNLQLLCFEVNAYNNEKFTLAGRRNVMNQLEREAKELAFGMPAREVDEIFRSQKEEFFFKGPRQQGGRSDE

SEQ ID NO: 41 - protein, 503 aa, Morus notabilis
MACLNKLSLVILSVLVLTASLASATQDPELKLKQCRHQCKHQRGFEEEQRQQCEQRCEEYIREKKHREREEEGYSRRSRESREEEEEKEESGERIEQNNPYLFEAEHFDTSVRTEEGRVHILKKFTERSKLLKGIENYRVGFLEANPHAFVAPVHFDADVIFFVASGRPTVTLLREEKRETFNLERGDVVHIPAGTPAYIVNRDEREKLFIVNLINPVYLPGEFESFHGPGGEDPESFYKAFSWKVLKAAFKRDRDEIERLFGQQKRGGITKASREQVEELSRHAEGGGGGGPIWPFKGETKGPFNLFRKHPVSSNRYGSLFEANTEDCKDLEDLNLMISFANITKGAMIAPNYNSKATKMSFVVDGEGWFEMACPHTPSEQGRQRGGGSAWQGEKSGKKFKRITGRLRRGMVFVVPAGHPVTAIASQNTNLQILCFEVNAKGNTRYPLAGRKNIVNNMDNVAKELAFGVPAREVERIFKSQNEEFFFPGPTQQEEGGHRAYM

SEQ ID NO: 42 - protein, 512 aa, Actinidia chinensis
MSLRGKLPLLGLLLALVFVFASVALGAAEWNDGSPEKRLRLRECQQGCERHERQEERGDCQRRCLEDYRREKEERGGHGRKYGERQRGEEETNPHHKGREEEEKESKEQQQGKPYVFEDQHFTTKLRTEHGRVQVLQKFTERSEILRGIENYRVAILEADPQTFIVPNHLDAETILYVAKGKGTVSMACPDKRESFNLKLGDVMRIPAGTTVYLINRSNNKKLVIVKLLQPVSTPGHFEAFFGPGGEDPESYYRAFSTELLEAALNTRKDKIQKVLGQQRRGVIIKASEEQIRAMSHQKEGGVWPFGGESKGPVNLFSKRAEQANQYGQLYEVDPPSECRELKELDLGVGFANITEGSMMGPFYNSRATKIAIVTKGQGYFEMACPHLSSSEFGGGKESQHEHQGKTGPRYQKVRAQLKRGMVYVVPAGHPIITVASNDQNLEIVCFDINAKNNKKFFLAGKTNVMNQLESEAKELGFGVPAREVDEVFRSQNEDLFFKGPRQQQHEGRADA

SEQ ID NO: 43 - protein, 501 aa, Parasponia andersonii
MAALKLSLLVLLLLSVVVLSSSVVWGKQDPELKLKQCQHQCRHQKGFDEEQKSQCERRCEDYIREKKERERRERERERGGGGGEEESYYGTREREEGERVTERGPYVFEEEHFETRVRTEEGRVHILRKFTEKSKLLKGIENFRVGFLEANPNAFVAPVHFDADTVFFVASGKPTVTLLREDKKDTFNLEFGDILNIPAGTPVYMVNRDEREKLFIIKLINPVSLPGHFEPFYGPGGENPETFYKAFSWEVLSAAFKRDRDEIERLFRQQKRGSIVKASRQQVQELSRHAEGGRGGGGTIWPFGGDTGGPFNLFRNRRPTHSNRFGSLFEADPSEYKQLEHLNLMISFANITKGAMTAPAYNSRATKISFVVDGEGFFEMACPHVSSEQGGGGRSRHSFERVTGRLRRGVVFVVPAGHPLTAITSGNTNLQILCFEVNAKGNVKYPLAGRKNIVNDMENVAKELSFSVPAREVDRIFRSQNEEFFFPGPTQQEQGGHRAYI

SEQ ID NO: 44 - protein, 504 aa, Trema orientalis
MAALKLSLLVLLLLSVVVLSSSVVWGKQDPELKLKQCQHQCRQQRGFDEEQKSQCERRCEDYIREKKERERRERESERGGGGGGGEEESYYGTKEREEGERVTERGPYVFEEEHFETRVRTEEGRVHILRKFTEKSKLLKGIENFRVGFLEANPNAFVAPVHFDADTVFFVASGKPTVTLHREDKKDTFNLEYGDILNIPAGTPVYMVNRDEREKLFIIKLINPVFLPGHFEPFYGPGGENPETFYKAFSWEVLSAAFKRDRDEIERLFRQQKRGSIVKASRQQVQEVSRHAEGGRGGGGTIWPFGGDTGGPFNLFRNRRPTHSNRFGSLFEADASEYKQLEDLNLMISFANITKGAMTAPIYNSRATKISIVVDGEGFFEMACPHVSSEQGGGGRSSRHSRQRVTGRLRRGVVFVVPAGHPLTAIASGNTNLQILCFEVNAKGNVKYPLAGRKNIVNDMENVAKELSFSVPAREVDRIFRSQNEEFFFPGPTQQEQGGHRAYI

SEQ ID NO: 45 - protein, 582 aa, Punica granatum
MAIKGLKFSLAVVLLTSLVVLSALSAATAKTDPELKQCQHQCKHQERFMERQQEQCEQRCGDYYRQKERERDSEEGGSSSGRGKESSRGDERKQLRECQRQCERQQGSSQQKQACRNQCQKRFGEKETDPWSREVEKREIGNRREEEQEEESWEEEQEEEISEENNPYVFQQEHFSLKVHTEHGRVRLLPRFTKRSKLLRGIENYRLGVLEANPQTFVVPNHWDADGVLFVISGRGTLTMVQENKRNSINVGKGDIIRVPAGTPVYLANRDENEQLHIANFLQPVNTPGQFEVYHATGGKNPEAFYTAFSWEVLEAAFKTGRDKLERMFRQQQQGAIVKASKEQIEAMSQADEEGSHWPFGGDEELGRLWPTRGSRGSHSYSFNIFNKRPSESNQYGQLYEVDQDDFKHLKDLDLMVSFANITRGSMTTLYYNSRATKIAMVVDGEGWLEMACPHLSSRQQGRRHSTRGQRRTGSPTTYQKVQARLNRGTVFIVPAGHPATSIASRNSNLQVVCFEVNAKNNVRYTLAGRRNIVGQMHREAKELAFNFPARDIDRIFKNQDEEFFFPGPARQEEVIRRYGDA

SEQ ID NO: 46 - protein, 486 aa, Arabidopsis thaliana
MAITNKLIITLLLLISIAVVHCLSFRVEIDEFEPPQQGEQEGPRRRPGGGSGEGWEEESTNHPYHFRKRSFSDWFQSKEGFVRVLPKFTKHAPALFRGIENYRFSLVEMEPTTFFVPHHLDADAVFIVLQGKGVIEFVTDKTKESFHITKGDVVRIPSGVTNFITNTNQTVPLRLAQITVPVNNPGNYKDYFPAASQFQQSYFNGFTKEVLSTSFNVPEELLGRLVTRSKEIGQGIIRRISPDQIKELAEHATSPSNKHKAKKEKEEDKDLRTLWTPFNLFAIDPIYSNDFGHFHEAHPKNYNQLQDLHIAAAWANMTQGSLFLPHFNSKTTFVTFVENGCARFEMATPYKFQRGQQQWPGQGQEEEEDMSENVHKVVSRVCKGEVFIVPAGHPFTILSQDQDFIAVGFGIYATNSKRTFLAGEENLLSNLNPAATRVTFGVGSKVAEKLFTSQNYSYFAPTSRSQQQIPEKHKPSFQSILDFAGF

SEQ ID NO: 47 - protein, 562 aa, Oryza sativa
MATRARATILLLLAAVLFAAAAAASGEDRRRETSLRRCLQRCEQDRPPYERARCVQECKDQQQQQQERRREHGGHDDDRRDRDRRGEGSSEEEDEGRERGSRRRPYVFGRRSFRQVVRSDQGSVRLLPPFHQASSLLRGIKNYRVAVLEANPRSFVMPTHTDAHCICYVAQGEGVVAIIENGEKWSYAIRQGDVFVAPAGTINYLANTDGRRKLIVTKILHTISVPGQIQFFFAPGGRNPESFLSSFSKGVQRAAFKISEEKLEKLLGKQDKGVIIRASEEQVRELRRHASEGGHGPHWPLPPFGESSRGPFNILEQRPRFANRHGRLYEADARSFHDLAEHDIRVAVVNITAGSMNAPFYNTRSVKVAYVLDGEGEAEIVCPHLSRGGRGGESEERRRERGKGKWREEEEEEEEQQKGQEEEEEEQVGQGYETIRARLSRGTVFVVPSGHPIVVTSSRDSTLQIVCFDVHANNNERMYLAGMNSVLKKLDPQAKELAFAASAREVDELLNAQQESAFLAGPEKSGRRGEESEDEDRRRRRSHRGRGDEAVETLLRMAAAAV

SEQ ID NO: 48 - protein, 574 aa, Zea mays
MVSARIVVLLAVLLCAAAAVASSWEDDNHHHHGGHKSGRCVRRCEDRPWHQRPRCLEQQCREEEREKRQERSRHEADDRSGEGSSEDEREREQEKEEKQKDRRPYVFDRRSFRRVVRSEQGSLRVLRPFDEVSRLLRGIRDYRVAVLEANPRSFVVPSHTDAHCIGYVAEGEGVVTTIENGERRSYTIKQGHVFVAPAGAVTYLANTDGRKKLVITKILHTISVPGEFQFFFGPGGRNPESFLSSFSKSIQRAAYKTSSDRLERLFGRHGQDKGIIVRATEEQTRELRRHASEGGHGPHWPLPPFGESRGPYSLLDQRPSIANQHGQLYEADARSFHDLAEHDVSVSFANITAGSMSAPLYNTRSFKIAYVPNGKGYAEIVCPHRQSQGGESERERGKGRRSEEEEESSEEQEEVGQGYHTIRARLSPGTAFVVPAGHPFVAVASRDSNLQIVCFEVHADRNEKVFLAGADNVLQKLDRVAKALSFASKAEEVDEVLGSRREKGFLPGPKESGGHEEREQEEEEREERHGGRGERERHGREEREKEEEEREGRHGRGRREEVAETLLRMVTARM

SEQ ID NO: 49 - protein, 523 aa, Amborella trichopoda
MASSWILTALLFLFVVVSPLLALSKRDPERERQERQCKSECERSREREREVRQCKQECEERYRRQKGQGRNIEERERESESESEGRRRGVTGNPYAFERDRFVRDISTEHGSFRTLPPFTERSELFRGIENNRICIMELKPNAFVLPHHKDADIIYYVANGDARLVTIGEERQDSHNLKKGDTATIPAGKTTYIINKDSQRELKIVALLQTISTPGQYEIFFGATGQNPPSYLRAFSDEILEASFNVDRSRLSRLLEQKGQGPIVRPSSEQLQGLIHGGSGGGKWPFGESEESERPFNLFSRRPRISNDHGELYELDETEYSPLREFDIAISFANISRGSLEVPFYNSRATEIYVILEGRARIEMACPHVSGGREQEGSRRGQGEQEGSRRGQREQEGSRREQEQEERGKGQGQEIKYHKIRSDLNPEDLFVAPPSHPIAIRASQEENLQIICFEINARRNRKYFLAGKNSILNQIERAAKEVSFNVPSREAEEVLRAQSDSVFVVGPRQQREGREGEGRAVA

SEQ ID NO: 50 - protein, 417 aa, Selaginella moellendorffii
MARKSLILCALALLIVSSSASEHDRHERHDRGDEEGDHHKQATSPFVLKEPVQVVSTEAGDIQVLPGPKELGALAENHIGLSIITIEPKGLLLPQYLDASQVLYVHKGRMTLGWAEQDYLNRQDLETGDIYALPGGFVFYVLNTDEGQRLRLYGMCDTSESLDAGHFQSFFVGGGVDPRTILSGFRKEAVAAALKVAPEDVSEILGSQTEGPIVYTSRAGYDFLKGDSAASNARGDAPKPFNLLKKAPDFKNENGWTIAVHGAEFSPLKEADVGVFAVSLKPGAMLAPHWNPRAAEVAFITKGNGRIQVSYPNGTNALDKELDESKVVFVPRYFPMCQIASRNGDFEFVGFSTSSRRNRPQFLAGSNSVFKAFSKDIMSKTFNNVDAKRLEAVLDNQRDAVILPGFIEEDGKRSSAM

SEQ ID NO: 51 - protein, 18 aa, Glycine max
PRRHKNKNPFHFNSKRFQ

SEQ ID NO: 52 - protein, 18 aa, Glycine max
LRRHKNKNPFLFGSNRFE

SEQ ID NO: 53 - protein, 19 aa, Glycine max
KNPQLRDLDVFLSVVDMNE

SEQ ID NO: 54 - protein, 19 aa, Glycine max
KNPQLRDFDILLNTVDINE

SEQ ID NO: 55 - protein, 19 aa, Glycine max
KNPQLRDLDIFLSIVDMNE

SEQ ID NO: 56 - DNA, 1883 bp, Glycine max
  source 1..1883: mol_type = genomic DNA
  unsure 388..487
acttttagcactttttcttaggcactagtttttttttttaacttaattttattctataagcacttaaaatcgccacaaatattctagatatatgtttttctatatgcaattttttacactcaacgcttggtctccaccctcactttcagatttggagtctccatatttgttgctttttaccaagaaaactcacatttgttgcattctcaatcttgtttgacatcttaaaaaaaatattgtttttgacaaccctcaataatatgtgatatttgaaatatatatatatatatatatatatatatatatatatatatatatatatatatatatatatatatatatatatatatatatatatatatatatatatatatatatatatatatannnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnncaactattatcgcatgatgatgtacgttaagtcatcatcatctttaactttatatattgttaaaagtagaaaaaataggtgatgcattataaaataattttataacatcatttaattataaattatttataataaatatttgagtttttatagtaattacctaaacaattatatcaagactaatgcctgattagttgacatgacgaaattaaactcataaaagtaaagatgtttatgtggaaaactcttatacaattgagcggacttttttccatggtagttcagttttcttctattcaatttatttttttggtttccgctcagaataagaataatttgataaattcatttttaggcaattaagaatatttatttgactaactttttaattgaaataaatttacaataaatactcaatttatctttcacaatcaaaagattgagatgttgtaagatctccgataatatacttatatcttttcatttattacgttttcaaatttgaattttaatgtgtgttgtaagtataaatttaaaataaaaataaaaacaattattatatcaaaatggcaaaaacatttaatacgtattatttaagaaaaaaatatgtaataatatatttatattttaatatctattcttatgtattttttaaaaatctattatatattgatcaactaaaatatttttatatctacacttattttgcatttttatcaattttcttgcgttttttggcatatttaataatgactattctttaataatcaatcattattcttacatggtacatattgttggaaccatatgaagtgtccattgcatttgactatgtggatagtgttttgatccaggcctccatttgccgcttattaattaatttggtaacagtccgtactaatcagttacttatccttcctccatcataattaatcttggtagtctcgaatgccacaacactgactagtctcttggatcataagaaaaagccaaggaacaaaagaagacaaaacacaatgagagtatcctttgcatagcaatgtctaagttcataaaattcaaacaaaaacgcaatcacacacagtggacatcacttatccactagctgatcaggatcgccgcgtcaagaaaaaaaaactggaccccaaaagccatgcacaacaacacgtactcacaaaggtgtcaatcgagcagcccaaaacattcaccaactcaacccatcatgagcccacacatttgttgtttctaacccaacctcaaactcgtattctcttccgccacctcatttttgtttatttcaacacccgtcaaactgcatgccaccccgtggccaaatgtccatgcatgttaacaagacctatgactataaatatctgcaatctcggcccaggttttc

SEQ ID NO: 57 - protein, 621 aa, Glycine max
MMRARFPLLLLGVVFLASVSVSFGIAYWEKQNPSHNKCLRSCNSEKDSYRNQACHARCNLLKVEEEEECEEGQIPRPRPQHPERERQQHGEKEEDEGEQPRPFPFPRPRQPHQEEEHEQKEEHEWHRKEEKHGGKGSEEEQDEREHPRPHQPHQKEEEKHEWQHKQEKHQGKESEEEEEDQDEDEEQDKESQESEGSESQREPRRHKNKNPFHFNSKRFQTLFKNQYGHVRVLQRFNKRSQQLQNLRDYRILEFNSKPNTLLLPHHADADYLIVILNGTAILTLVNNDDRDSYNLQSGDALRVPAGTTYYVVNPDNDENLRMITLAIPVNKPGRFESFFLSSTQAQQSYLQGFSKNILEASYDTKFEEINKVLFGREEGQQQGEERLQESVIVEISKKQIRELSKHAKSSSRKTISSEDKPFNLRSRDPIYSNKLGKLFEITPEKNPQLRDLDVFLSVVDMNEGALFLPHFNSKAIVVLVINEGEANIELVGIKEQQQRQQQEEQPLEVRKYRAELSEQDIFVIPAGYPVVVNATSDLNFFAFGINAENNQRNFLAGSKDNVISQIPSQVQELAFPGSAKDIENLIKSQSESYFVDAQPQQKEEGNKGRKGPLSSILRAFY

SEQ ID NO: 58 - DNA, 2730 bp, Glycine max
  source 1..2730: mol_type = genomic DNA
  5'UTR 1..58
  3'UTR 2549..2730
atcatcaagaaccagttcaatatcctagtacaccgtattaaagaatttaagatatactatgatgagagcgcggttcccattactgttgctgggagttgttttcctggcatcagtttctgtctcatttggcattgcgtattgggaaaagcagaaccccagtcacaacaagtgcctccgaagttgcaatagcgagaaagactcctacaggaaccaagcatgccacgctcgttgcaacctccttaaggtggaggaagaagaagaatgcgaagaaggtcaaattccacgaccacgaccacaacacccggagagggaacgtcagcaacacggtgagaaggaggaagacgaaggtgagcagccacgtccattcccattcccacgcccacgccaacctcatcaagaggaagagcacgagcagaaggaggaacacgaatggcatcgcaaggaggaaaaacacggaggaaagggaagtgaagaggaacaagatgaacgtgaacacccacgcccacaccaacctcatcaaaaggaagaggaaaagcacgaatggcaacacaagcaggaaaagcaccaaggaaaggaaagtgaagaagaagaagaagaccaagacgaggatgaggagcaagacaaagagagccaagaaagtgaaggttctgagtctcaaagagaaccacgaagacataagaataagaacccttttcacttcaactctaaaaggttccaaactctcttcaaaaaccaatatggccacgttcgcgtcctccagaggttcaacaaacgctcccaacagcttcagaatctccgagactaccgcattttggagttcaactccaaacccaacacccttcttctcccccaccatgctgacgctgattacctcatcgttatccttaacggttagtattcctttcctctcaaaataaaataaatgtcattattttatgcggattaagaaaaattattaaataaataaaaacaaaaccaacaattttataaaaactaatcttactatgacacttatttaattaattaataaacatcatcacatgcttttacttctcataaaattatgttattgaatttgttttgttaattacagggactgccattcttaccttggtgaacaacgacgaccgagactcttacaaccttcaatctggcgatgccctaagagtccctgcaggaaccacatactatgtggttaaccctgacaacgacgagaatctcagaatgataacactcgccatacccgttaacaaacccggtagatttgaggtactacttattctttataactaattaattaattattattctaatttgtttccaaatcttaagatcaatctttttcttttctctgcagagtttcttcctatctagcactcaagctcaacagtcctacttgcaagggttcagcaagaatattctagaggcctcatacgacgtaagcaaccaacacaccatctaatacgctagcaaattcaatattatcattatccttatatttgtttccgcgcttgattttatagaccaaattcgaggagataaacaaggttctgtttggtagagaggaggggcagcaacaaggggaggagaggctgcaagagagtgtgattgtggaaatctcaaagaaacaaattcgggaactgagcaaacatgccaaatctagttcaaggaaaaccatttcttctgaagataaacctttcaacttgagaagccgcgaccccatctattccaacaagcttggcaagttgtttgagattaccccagagaaaaaccctcagcttcgggacttggatgtcttcctcagtgttgtggatatgaacgaggtaagcagaaaaactgaacatgacaattgagttctattcactttcttcttagttagagaaacctattcttgagactttaaataatgatttacttttttctttgttcacaaatatagggagctctttttctgccacacttcaattcaaaggccatagtggtactagtgattaatgaaggagaagcaaacattgaacttgttggcattaaagaacaacaacagaggcagcaacaggaagagcaacctttggaagtgcggaaatatagagctgaattgtctgaacaagatatatttgtaatcccagcaggttatccagttgtggtcaacgctacctcagatctgaatttctttgcttttggtatcaatgccgagaacaaccagaggaacttccttgcaggtacatattttgtatatcacgatcaaatagaacatgctgttgaagtgttgttactttttttgttttattaattacatgtgaaacatagctgactgagctatttctaatcctttggtgattgaaaatttgaaggttcgaaagacaatgtgataagccagatacctagtcaagtgcaggagcttgcgttccctgggtctgcaaaagatattgagaacctaataaagagccaaagtgagtcctactttgtggatgctcagcctcagcagaaagaggaggggaacaagggaagaaagggtcctttgtcttcaattttgagggctttttactgaataagtatgtagtactaaaatgtatgctgtaatagctcatagtgagcgaggaaagtatcgggctatttaactatgacttgagctccatctatgaataaataaatcagcatatgatgcttttgttttgtgtacttcaactgtctgcttagctaatttgatatggttggcacttggcacgtata

SEQ ID NO: 59 - DNA, 1866 bp, Glycine max
  source 1..1866: mol_type = other DNA
atgatgagagcgcggttcccattactgttgctgggagttgttttcctggcatcagtttctgtctcatttggcattgcgtattgggaaaagcagaaccccagtcacaacaagtgcctccgaagttgcaatagcgagaaagactcctacaggaaccaagcatgccacgctcgttgcaacctccttaaggtggaggaagaagaagaatgcgaagaaggtcaaattccacgaccacgaccacaacacccggagagggaacgtcagcaacacggtgagaaggaggaagacgaaggtgagcagccacgtccattcccattcccacgcccacgccaacctcatcaagaggaagagcacgagcagaaggaggaacacgaatggcatcgcaaggaggaaaaacacggaggaaagggaagtgaagaggaacaagatgaacgtgaacacccacgcccacaccaacctcatcaaaaggaagaggaaaagcacgaatggcaacacaagcaggaaaagcaccaaggaaaggaaagtgaagaagaagaagaagaccaagacgaggatgaggagcaagacaaagagagccaagaaagtgaaggttctgagtctcaaagagaaccacgaagacataagaataagaacccttttcacttcaactctaaaaggttccaaactctcttcaaaaaccaatatggccacgttcgcgtcctccagaggttcaacaaacgctcccaacagcttcagaatctccgagactaccgcattttggagttcaactccaaacccaacacccttcttctcccccaccatgctgacgctgattacctcatcgttatccttaacgggactgccattcttaccttggtgaacaacgacgaccgagactcttacaaccttcaatctggcgatgccctaagagtccctgcaggaaccacatactatgtggttaaccctgacaacgacgagaatctcagaatgataacactcgccatacccgttaacaaacccggtagatttgagagtttcttcctatctagcactcaagctcaacagtcctacttgcaagggttcagcaagaatattctagaggcctcatacgacaccaaattcgaggagataaacaaggttctgtttggtagagaggaggggcagcaacaaggggaggagaggctgcaagagagtgtgattgtggaaatctcaaagaaacaaattcgggaactgagcaaacatgccaaatctagttcaaggaaaaccatttcttctgaagataaacctttcaacttgagaagccgcgaccccatctattccaacaagcttggcaagttgtttgagattaccccagagaaaaaccctcagcttcgggacttggatgtcttcctcagtgttgtggatatgaacgagggagctctttttctgccacacttcaattcaaaggccatagtggtactagtgattaatgaaggagaagcaaacattgaacttgttggcattaaagaacaacaacagaggcagcaacaggaagagcaacctttggaagtgcggaaatatagagctgaattgtctgaacaagatatatttgtaatcccagcaggttatccagttgtggtcaacgctacctcagatctgaatttctttgcttttggtatcaatgccgagaacaaccagaggaacttccttgcaggttcgaaagacaatgtgataagccagatacctagtcaagtgcaggagcttgcgttccctgggtctgcaaaagatattgagaacctaataaagagccaaagtgagtcctactttgtggatgctcagcctcagcagaaagaggaggggaacaagggaagaaagggtcctttgtcttcaattttgagggctttttactga

SEQ ID NO: 60 - DNA, 2000 bp, Glycine max
  source 1..2000: mol_type = genomic DNA
aattcacagagatacatttaaactttttttagaaacttgcttagtatcttatcctgtttgttattcatttttggcagttggtcctaaagatactcctatgaatcttgtgctagagaagacttacgatgctaaaacaggacggggcatgcctgaactttaaggagacgttgccctgttccacttccaattaggtaactgctatcgtgatgaacaaaaatttggtgagtttatcaccttgccctttgccatgattcaattaaaagcgtgtttggactttggaacctcattctaacaccaccctatgatgggttagacgcaaaatctagactgggtagtgtttaacgtgtatctgtgtgaacacagttacaaacgcattccttgtttaatgctaccatgcctaggagttgaatcatttgtaactttaccaatttagtcattactactagcattcttttccctattcaagttgatgttagctccagttagtgatggtcatttcactctataaactttaattgttagatgagtggaagaggaacctgtttgattgttatggttctagttctagtgatttttattaattgggttcgaccatattagtgtttgatttgagctatagatagttttttccccaaaagatcagtcttctctcatgtcagattcatgggttggtactctttttatccagttccaacaaacttgctgttcgaactacgaagtcagtcttacttattgggtaacatgtgggttttggtgtttaatggatctagaatactgtttgtagctaaacctatcttatcataaagggcctaaaaagtaaaattggttattacatttggaaaaaaagaaataatctaggcccactggcacactgagaaacgttttcaatgaataatttaatagtttttttttataaaaaaatattaataaaaaataatggagtttttaaaaatattacaacaatctgtttctctaaggttttttaatagttcagataattcatagcttagagcaatacgacatggttaggaagcataaaaaaaatatacgacatggttaggaatttttttttagtatgtctgacataattttttaaatgttttggcttcatatgaatttaacagtgcgtcatatgaacttacacactcattatattttttaaccttttaaatgattttaaaaaaaatatgacagatgcaatcttattttcactttttatactttcactactgcttcatatgacctaaagtcagagaaatattttaaaaagataaatacgataaagaatacgatgagaaagaaacctcacacaatgaatagaccaaattagacctatttattttccttagaaataaagaaaataattattttttattttttcacattacatttatatttttctatcactttctctatttaggtattgattggcatatgagtgtacatgaatttttttttttaaaaaaagcgtaaatattaattatattcatgcattatttgttttctgtctttcattttctatttaatcttacgttatcaataatttattattaaattttatagttgatgatgaatatataagagatataaataaaaaaaataattaattttataataaaaattaaaaaataattattttgagataaaaaattttaagagaacaattataaacggagagtattatatttagttttatgtaccgtgtacgtgtctactaacatggtgtctctccatcattttcgtaggaaaaaacattataggagtatgaaaaaagcaaaagttttgtctgtttatggttttgtatatacccagctctacttggcagcaattacccgtcttgcttgctacttacgagacacgtacattaacacttgtcctagctagtgcatgcaattgccaccccattcatcactcctcccttttccttctctttatatttatatatatatatatatatatatatatatatatatatatatatatatatataaacaagcaca

SEQ ID NO: 61 - protein, 504 aa, Glycine max
MAMRTKLSLAIFFFFLLALFSNLAFGKCKETEVEEEDPELVTCKHQCQQPQQYTEGDKRVCLQRCDRYHRMKQEREKQIQEETREKKEEESREREEEQQEQHEEQDENPYIFEEDKDFETRVETEGGRIRVLKKFTEKSKLLQGIENFRLAILEARAHTFVSPRHFDSEVVFFNIKGRAVLGLVSESETEKITLEPGDMIHIPAGTPLYIVNRDENDKLFLAMLHIPVSVSTPGKFEEFFGPGGRDPESVLSAFSWNVLQAALQTPKGKLEKLFDQQNEGSIFAISREQVRALAPTKKSSWWPFGGESKPQFNIFSKRPTISNGYGRLTEVGPDDDEKSWLQRLNLMLTFTNITQRSMSTIHYNSHATKIALVIDGRGHLQISCPHMSSRSSHSKHDKSSPSYHRISSDLKPGMVFVVPPGHPFVTIASNKENLLMICFEVNARDNKKFTFAGKDNIVSSLDNVAKELAFNYPSEMVNGVFDRKESFFFPFELPREERGRRADA

SEQ ID NO: 62 - DNA, 3245 bp, Glycine max
  source 1..3245: mol_type = genomic DNA
  5'UTR 1..55
  3'UTR 3000..3245
atgcatcatctcaaagaaattaagagagttttttttgttcctcactgaccaagccatggcgatgagaaccaaactttctttagctatctttttcttttttcttttagccttattttcaaacctagcctttggcaaatgtaaagaaaccgaggtagaagaagaagaccctgagctcgtaacatgcaaacaccaatgccaacagccacagcaatacactgagggtgacaagcgggtatgcttgcaaagatgtgacaggtatcatcgtatgaagcaagagcgagagaaacaaatccaagaggaaactcgtgagaagaaagaggaggaaagccgtgaaagggaagaagaacaacaagagcagcatgaggaacaagatgaaaatccctacatttttgaggaagataaggattttgagaccagagtcgaaactgaaggtggcagaattcgggttctcaagaagttcactgaaaaatccaagcttcttcagggcattgagaacttccgtttggccattttggaagctagagcacacacgttcgtgtctcctcgccactttgattccgaggttgtttttttcaacatcaagggtacgtaaatttaaattaagcaaaatatatattctttttagtttaagatatttgcatatgagttttgacttctgagtttaatgttgatgcatgcatttactaaccatttaaaataattttatacttttattcaatcataaaatattatttttataacttttaaaataattattatatttatataataataagttataattagacgtcatataaaattattttacactatattttttcttttatatatatatatatataatattaatttattgatgctttttttattaatttgttggtgattcacacatctactgttgatgctgtacaattttgtgaaatttccttacttcatttgttgtatttctatttatttactagggcgagccgtacttgggttggtgagtgaaagtgaaacagaaaaaatcacccttgaacctggggacatgatacacataccagctggcaccccactgtacatcgttaacagagatgagaatgataagctcttccttgccatgctccatatacctgtctctgtctctactcctggaaaatttgaggtaattgcttctaattatatatacatatggattagttaagtttttaataacatgcaaaagtctagagaactagaccaaacaaagttttgatcattaattaaaaatcaaatttcaaaatctttagaaactaaatcaatactcacaattgagttcacatgttaactaattaaagcaagtagaagatggtatatacgatatagttgaatattatattttatactgatcgaaggaaattgtggtgtaggaatttttcgggcctggaggacgagacccagaatcggtcctctcagcattcagctggaatgtgctgcaagctgcgctccaagtatatagacccttaatttctagctatcatgcatcaatattatattatagcataaagtaacattaaatgctactaatttggcttgtcaatttcattaacttggtttcttgtattttttaccaaaatggaaaataattacagaccccaaaaggaaagttagaaaagctttttgatcaacagaacgagggaagtattttcgcaataagcagagaacaggtgcgtgcgttggcccctaccaagaaaagctcttggtggccattcggtggcgaatccaagcctcaattcaatattttcagcaagcgtcccactatctccaacggatatggtcgtttaactgaagttggtcctgacgatgatgaaaagagttggcttcaaagactcaatctcatgcttacctttaccaacatcacccaggtatattatcaacacgcttatcttatctttatttatttatctcattaactgatgagtaagttactagtttttgaactgttatcaactctaatgtagactacgtttattaatttcaaacaataattctcttaattggttgtcatttttttatcagtttggttaatatcttggttgccatctatagtaaatagaacttaaattcttggtaatctttcaagaaaaaacagtacatgtatatatagtgtaaagatcattgacattatcaaccaatttgatattgttcaatggtagtctttttggctttatcataattaaatttttcttaacatgaaaacaagccttgtgactgataacattatattatttccataagatattgtcatggatggtagctagttttttcttacttttataataattaacttattctttaagatgataacgtgatctgggactgatcttgacagtttaaaatttactgcatagatttgaactcaattcttaataatgatttgcatgttctcctataatttaatagagatctatgagtactattcactacaactcacatgcaacgaagatagcactggtgatcgatggcagagggcatcttcaaatatcatgtccacacatgtcatcaaggtcgtcacactcgaagcatgataagagtagcccttcataccatagaatcagttccgatttgaagcctggaatggtgtttgttgtccctcccggtcatcccttcgtcaccatagcttccaataaagagaatctcctcatgatttgcttcgaggttaacgctcgagacaacaagaagtttacatttgcaggtaggcacacacacataaacccttagttatgccatgcatacattttttttatgcatgcatgttcatttggtttttaattagtttattgcaaagttgaagctagttattatatatattgtgtgtaacagggaaggacaacattgtgagttctctggacaacgttgctaaggagcttgcctttaactatccttctgagatggtgaacggagtattcgaccgaaaggagagtttctttttcccttttgagttgccgagagaggagcgtggtcgtcgcgctgatgcgtgagaggtttagaacaatcaagaaaaggtgtgcatgtggctgaagatcacggggaatgtattaagcttcagagactctttaaattaaattttctgtattttgtgttatatgttactagttctttaaattagccagatggagtttatgtgtatctaaatgcagggatgctaatggaataaaatggccacttgtattgttagctatctcttatggtagcagaataagacgtaaactggttctttgctccaa

SEQ ID NO: 63 - DNA, 1515 bp, Glycine max
  source 1..1515: mol_type = other DNA
atggcgatgagaaccaaactttctttagctatctttttcttttttcttttagccttattttcaaacctagcctttggcaaatgtaaagaaaccgaggtagaagaagaagaccctgagctcgtaacatgcaaacaccaatgccaacagccacagcaatacactgagggtgacaagcgggtatgcttgcaaagatgtgacaggtatcatcgtatgaagcaagagcgagagaaacaaatccaagaggaaactcgtgagaagaaagaggaggaaagccgtgaaagggaagaagaacaacaagagcagcatgaggaacaagatgaaaatccctacatttttgaggaagataaggattttgagaccagagtcgaaactgaaggtggcagaattcgggttctcaagaagttcactgaaaaatccaagcttcttcagggcattgagaacttccgtttggccattttggaagctagagcacacacgttcgtgtctcctcgccactttgattccgaggttgtttttttcaacatcaaggggcgagccgtacttgggttggtgagtgaaagtgaaacagaaaaaatcacccttgaacctggggacatgatacacataccagctggcaccccactgtacatcgttaacagagatgagaatgataagctcttccttgccatgctccatatacctgtctctgtctctactcctggaaaatttgaggaatttttcgggcctggaggacgagacccagaatcggtcctctcagcattcagctggaatgtgctgcaagctgcgctccaaaccccaaaaggaaagttagaaaagctttttgatcaacagaacgagggaagtattttcgcaataagcagagaacaggtgcgtgcgttggcccctaccaagaaaagctcttggtggccattcggtggcgaatccaagcctcaattcaatattttcagcaagcgtcccactatctccaacggatatggtcgtttaactgaagttggtcctgacgatgatgaaaagagttggcttcaaagactcaatctcatgcttacctttaccaacatcacccagagatctatgagtactattcactacaactcacatgcaacgaagatagcactggtgatcgatggcagagggcatcttcaaatatcatgtccacacatgtcatcaaggtcgtcacactcgaagcatgataagagtagcccttcataccatagaatcagttccgatttgaagcctggaatggtgtttgttgtccctcccggtcatcccttcgtcaccatagcttccaataaagagaatctcctcatgatttgcttcgaggttaacgctcgagacaacaagaagtttacatttgcagggaaggacaacattgtgagttctctggacaacgttgctaaggagcttgcctttaactatccttctgagatggtgaacggagtattcgaccgaaaggagagtttctttttcccttttgagttgccgagagaggagcgtggtcgtcgcgctgatgcgtga

SEQ ID NO: 64 - DNA, 2000 bp, Glycine max
  source 1..2000: mol_type = genomic DNA
acatgatttcccccccttggtatcaactaaagcatgttggacttgcgctccatatgcagaaactcaaattaaaaacatcatttgtaatgtatagtaagtgtatatataacattgtaagttgtcgatcaaagttatttggattaatggatttaagtcttctataatattccattgagagccagaagccaggtccaaaggaataggtaattcgcatgaattcattctcttgcttctatacagctatttttccatcttagtgttgcgggaaactacttcagttcttgcagatgtgcaaaacttgtagggatccatgtagttcagtgaaacccatgctttcttaattgacagagatacattaaaactttttacagaattgagaaacccaagccttgttaattctcaaagatacatttaaacttttttcagaaacgtgctgagtattttatcctgtttgttattcatttttggcagttggtcctaaaaatactcctatgaatcttgtgctagagaagacttgcaatgctaaaacaggacggggcatgcctgaactttaaggagacgttgccttgttccgattaggtaattgctatcgtgatgaacaaaaatttggtgtgaatttatctccttgccctttgccatgattcaattaaagacgtgtttggaaccacattctaacaccactttatgatgggttagacgcaaaatctagattgggtagtgtttacacacagttacaaacacattccttgtttaatgttatcatgcctaggagttgaataacttgtaactttaccaattagacattactactagcattctttttcctattcaagttgatgttatctccagttagtgatggtcatttcattccataaacttcaattgttaaaatgagtgaaaagggaaaaaggaacccgtttgattgttatggttctagtgatttttattaattgggtttgtccattagtgttgatttgagctaaatagtttcccccccccaaaagatcagtcttctcacatgtcatattcatgcgctggtacccttttcatccagttccaacaaacttgctgtacgaagtcaggttgcatgaaaatagtcaaattttctttaagggggatattatacgtaaataaataacgtaacccaaaagtcttacttgttgggtaacgtgggttttggtgtttgatggacctagaacactgtttgttgctcttatatgcttacaaagtaaaaatggttatcacatttggggaaaaaatgtaggcccacttatgatatttcgacctaaatgcaaaatggtttatcaatttttttatacttagtataataaaactccttttttttttccactggcatactatttctctaagactttttaatagttccgataattcttagcttaaagaaatacgacaaggttaggaatatttttttattatgtgacattattttttaaatattttgcttcatatgaatttatacaatcattataatttgaccttttaaatgacttttaaaaatgatcagacctaaaatttgagtcttctgattgagatgcaaacttatttctttttatattttatattttatactcatttgtttctctttctattatatttcttttttttcttctctttatgcaaaaacgtatgacgttgattggtgtctttggcaatctttttatgacgctcaaaagtgaaaataaatattgttcactttcacctcacgctggccttccgctgatggtggttgtacgcacttatttgatttttttttcttccacatttaatgaggtgaatcagttagagaaatattaaaaaaaataaataaataaaagaagacgactaatacaataaagaatacgaaactcacaatgaatagacccaattggaaccatttattttccttacaaattaaagaaaacatttttttaacaatatatcacattatcatctattatatttttatttatattttttataactttctctatctaggt

SEQ ID NO: 65 - protein, 506 aa, Glycine max; identical to SEQ ID NO: 17

SEQ ID NO: 66 - DNA, 4055 bp, Glycine max
  source 1..4055: mol_type = genomic DNA
  5'UTR 1..162
  3'UTR 3808..4055
gtagattgacatgagtatacgcacgcacacccagctctacttagcagcaattacccgttttacttgctacttaagagacacgtacattaacacttgtccttgtgcatgcaattgccaccacattcctcactccacccttttctttatatataaacaaacacaatggatcatctcaaaccaagagtgagtttgttgttcctcactgacctcaccatggcgaccagagccaagctttctttagctatcttccttttctttcttttagccttgatttcaaacctagccttgggcaaacttaaagaaaccgaggtcgaagaagatcccgagctcgtaacatgcaaacaccagtgccaacagcaacggcaatacactgagagtgacaagcgaacatgcttgcaacaatgtgacagtatgaagcaagagcgagagaaacaagtcgaagaggaaactcgcgagaaggaagaagaacatcaagagcagcatgaggaggaggaagacgaaaatccctacgtttttgaagaagataaggatttttcgaccagagtcgaaacagaaggtggcagcattcgggttctcaagaagttcactgagaaatccaagcttcttcaaggcattgagaatttccgtttggccatcttagaagctagagcacacacgttcgtgtccccacgccactttgattccgaggttgtcttgttcaacattaagggtgcgtatatttgaattaaacaaaaaatacatttttttttgttagtttcagttatgtacccgagtttaatagtgattacactctaattttatatcattattcaatcatacattaatatttatatgatttttagaataattattttttttaatgaaactttatgataggtttttgtcttagatgacaatatatcaattaatttattgatgccttttttattattaatttgttggttattcacacatctactgctcatgctgtacaattttgtgaaatttcctcctttcattattagttgtattactatttatttactagggagagccgtacttgggttggtgagggaaagtgaaacagaaaaaatcaccctagaacctggagacatgatacacataccagcaggcacaccactgtacatcgttaacagagatgagaatgagaagctcctccttgccatgctccatatacctgtctctactcctggaaaatttgaggtaattatatatacataagtgctagttatgcttttagtttttaaatttttatccgttaagatttttttctgaacaaaagtttgactttgacttcagtctacaattttttaaaaatttgatttttaatttatgaagagaagtttgaatccttttattttcatttgtttaacagatttttttagatgaaaatcgaatttaaaaaaaaaagtttaagactgcttaaacttttgttcaagaacgaacaatcttaataggaaaaaattaaaaaaactagaccaatcaatttttttttactaagaaatgaatttaaaaaatctttaaaaactaaagacttagttaatcttgtacacacatacatgtacatcaactttacaatttacaaacttaattcagtacatttatgaatatacatgtatgtatatgtgtgtgtgtgtatatatatatataaactttacaatttacaaacttaattcagattgactaaatcaatactcacgattgagttcacacatgttaactaattaaacaaagtagaagatagtatatagttgaatattatatttaaatttatactgatggaagaaaattgtggtgtaggaatttttcgggcctggaggacgagacccagaatcggtcctctcagcattcagctggaatgtgctgcaagctgcgctccaagtatatagacccttaatttcttgctatcatgcatcattaacattaaatgctactaattaatttggctttgtcaatttgatttctttttattttgggaaattaattacagaccccaaaaggaaagttagaaaggctttttaatcaacagaacgagggaagtattttcaaaataagcagagaacgggtgcgtgcgttggcccccaccaagaaaagctcttggtggccattcggcggcgaatccaaggctcaattcaatattttcagcaagcgtcccactttctccaacggatatggccgtttaactgaagttggtcctgatgatgaaaagagttggcttcaaagactcaacctcatgcttacctttaccaacatcacccaggtatatatattaccaacatgcttatcttaattttttttacttgatagtcataaggaaaagattgtgagtttgatttcatccgctaataacatactaataatttaataactaatatttactgattaattaaaaaaatgtttattaacttcaaagaaaatttatcttggttgtcatttttttatcagttcagtttggtttatatattggttgtcatctagtacataaaacttaaattctttgtaatttttcaagagaaaacagtacatgtatagtgtaattaagtagcatttacattatcaaccaataagatatttcttattgtatggatggtatagtctttttgactttataataattaactttttcataacatgataacattatttgtgactgatagatagaataattacaataagatatcgttttagatggttgtctttttgaataataattaacttattcttaacatcataacatgatctgtgactttaaaatttactgcatagatttgaactcaattcttaattgataactaataataatgatgttacatgttatcctaattaaatagagatctatgagtactattcactacaactcacatgcaacgaagatagcactggtgatggatggtagagggcatcttcaaatatcatgtccacacatgtcatcaaggtcagactcaaagcatgataagagtagcccctcataccatagaatcagtgcggacttgaagcctggaatggtgtttgttgtccctcctggtcatcccttcgtcactatagcttccaataaagagaatctcctcataatttgcttcgaggttaacgttcgagacaacaagaagtttacgtttgcaggtaggagtacacataaatccttaataatgccatgcatacatttttttttaacgtgtagggatcaaagaataattcacgtatctttttaaccaattaaattaaaaaagatatattataacaattgaactattgtgaagcataaaagaacaaaattcttatttagttttaaagcaataaaaatatgcaaataattgaaaaggtgaaagaaagagagaaaaatatataatgatataaaattctaatatatatctaaataaaaatatacagataaatgaaaaggtgaaagaaagagagaaaaatatatcaatctaaattaatttatatctatttatatatgaattaatttataacttaaacaaattaagaggtctgaatcaataaaaattcaaaattgagaatattgattaaaaaaaacaaatgttaattttacgaactgaatttaaccctcttatttgtcatgtgtcatgcatgcatgttgatttggttcataacgagttgaagctaattatatatattgtgtgtggaacagggaaggacaacattgtgagctctctggacaacgtagctaaggagctggcctttaactatccttctgagatggtgaacggagtcttcgaaagaaaggagagtctctttttccccttcgagttgccgagcgaggagcgtggtcgtcgcgctgttgcgtgagaagcagtgtggaggtggctgataacggggaatgtatttagctttgagagtctttaaattttctgtatttgttgtaatgttagtagttccttaaattggccagatggagtttatgtgtttgtaaatgcagggatgctaacggaataaaatggccacttgtattgctatcccttgtgctagcataatagaaagggttgaatcgagctaatccgagtttcattcagtttaactcgagttttttttaaaat

SEQ ID NO: 67 - DNA, 1521 bp, Glycine max
  source 1..1521: mol_type = other DNA
atggatcatctcaaaccaagagtgagtttgttgttcctcactgacctcaccatggcgaccagagccaagctttctttagctatcttccttttctttcttttagccttgatttcaaacctagccttgggcaaacttaaagaaaccgaggtcgaagaagatcccgagctcgtaacatgcaaacaccagtgccaacagcaacggcaatacactgagagtgacaagcgaacatgcttgcaacaatgtgacagtatgaagcaagagcgagagaaacaagtcgaagaggaaactcgcgagaaggaagaagaacatcaagagcagcatgaggaggaggaagacgaaaatccctacgtttttgaagaagataaggatttttcgaccagagtcgaaacagaaggtggcagcattcgggttctcaagaagttcactgagaaatccaagcttcttcaaggcattgagaatttccgtttggccatcttagaagctagagcacacacgttcgtgtccccacgccactttgattccgaggttgtcttgttcaacattaaggggagagccgtacttgggttggtgagggaaagtgaaacagaaaaaatcaccctagaacctggagacatgatacacataccagcaggcacaccactgtacatcgttaacagagatgagaatgagaagctcctccttgccatgctccatatacctgtctctactcctggaaaatttgaggaatttttcgggcctggaggacgagacccagaatcggtcctctcagcattcagctggaatgtgctgcaagctgcgctccaaaccccaaaaggaaagttagaaaggctttttaatcaacagaacgagggaagtattttcaaaataagcagagaacgggtgcgtgcgttggcccccaccaagaaaagctcttggtggccattcggcggcgaatccaaggctcaattcaatattttcagcaagcgtcccactttctccaacggatatggccgtttaactgaagttggtcctgatgatgaaaagagttggcttcaaagactcaacctcatgcttacctttaccaacatcacccagagatctatgagtactattcactacaactcacatgcaacgaagatagcactggtgatggatggtagagggcatcttcaaatatcatgtccacacatgtcatcaaggtcagactcaaagcatgataagagtagcccctcataccatagaatcagtgcggacttgaagcctggaatggtgtttgttgtccctcctggtcatcccttcgtcactatagcttccaataaagagaatctcctcataatttgcttcgaggttaacgttcgagacaacaagaagtttacgtttgcagggaaggacaacattgtgagctctctggacaacgtagctaaggagctggcctttaactatccttctgagatggtgaacggagtcttcgaaagaaaggagagtctctttttccccttcgagttgccgagcgaggagcgtggtcgtcgcgctgttgcgtga

SEQ ID NO: 68 - DNA, 2000 bp, Glycine max
  source 1..2000: mol_type = genomic DNA
tgactaacgtcagctgaaaaatattttcggtatattttgacaaaaaaaaaaccctatttgatgtcgatcgaaaaatagtcatggttaatgttggtttaaaaacaccactgcttgtgatcagacaaaacaaccatagttaaaattatcaatattttgtatttttaaattattaaaaattgaaaaatattttttaattaatatttcaaaattacattgtgtttgttttctataaaatttaatattaaaatttcatctatttaaaatataaaattaaaattttacatatgaagaaaaatgaattatcctattcaaataaagaatttaaaattgaaaaaatttaaattaatttatttaaataaaacttttaaaaaatgaaagaaattaaaattaaaacaattcaaattctataaacatttcaaaattgcttatagagacacaagattactatctaccagaaaagcaatcaactttgttttcaattcttcaactgctcaattctgtgcttaattactttgatacgcatgctggttggcacggaattcaattaaaaaatgtttttttattacaattaaattaatacgaaaattttatgagaacaaaattaatatcttttaaaattctacaaaatatcaaatatattttattcataaatatttcaccatgtttgtaattaaacacgaaaatttgaactttaattaaataacatgctatcacttgcagttgggaaaataatcaactaattacgtttataaaattctagattgagtcctccttggttcttaccaagtcaggtaaacttgttttctccttacttagatccggcacgggcttaaaaagtaaagtataacatgaatcaagataaatctcttgcggcaaattaagttaagatactttaaattagtgataatttttttttaaaatatttgaaaaatagatcactcgtacaatcaagaatgtggatctattattgagagaattgaatcatgacttagtgatggtgaaggtctgagtagtttgcacaaatttttaagttctaaatttttttatcatcattgtaaaaacaatcatcattgagaaaattaaaagttttagatgaaaagcgtaattaaaattttaataatttatttttttaagaaaaatgaagagattcataattgggttatccatttaaatatcaaatgataatgaacaacattttactttaagagggggcggtgcctgcagatgtccttggacttgcccctgctctggctgccgaccaccgcaacctgagggaggtcgatggcggagcggctcccctcgcggctgaagatgtcctggagtgtagagagagaagaagcgtcgagaaaaagtgagcaacgtaaggtctcaatgtgaacatcaaggggtatgtaagatagagtaaatctaacaaaccaatttttattgtttttgaaataagggtaaaacaactttaaatctaacaacacaaattttgtcaaacacgtttttacagactgactgacaaccacccataggtactgttaagcaaccgcataacaccctacttcacttcacttcacttatccactaactgttcaagatcgccgcatccgtacgtatatatctaaaactaaaaggactggaccccaaatgccatgcacaacaacacgtattcaccaatgtgtccaccttgccgctcaaatcgctcataacctcatcccacgagtccaatgcgaaccaaccctaaataaataactaaaataagacccgtattcttttcaaccaccccaattttgttcaacaattcaacacacgtcactctgcatgcccatcctgtggccaaattttcatgcaagttaaccaaagtcgctttttctcttctgtaactatatatatatatatatatatatatatatatatagctctaatctcacaccgtgttttcatcatcacacaagagcgagcgcaacattattcgagtacgtagcttaacaatattttaagcaattgctgctaca

SEQ ID NO: 69 - protein, 596 aa, Glycine max; identical to SEQ ID NO: 18

SEQ ID NO: 70 - DNA, 2324 bp, Glycine max
  source 1..2324: mol_type = genomic DNA
atgatagcgcggtttcctttgttgccgttgctgttgctgggaattgtttttctagcatcagtttctgtctcattacaggtgaagccgccgagtcaccacgagtgcctccagagttgcgagagtgaaacggatccatacaagcggaaagcatgcaagcttagttgcaagttcgttcctgagcacggggagcaacaacatgaagaagaagaaagagaaagagaacatccacaaccacacccaccacacgaggagagaggacgtagagaaagaggacgccaggaaggtgagaaggaggaaaaggaacaagacgagcgtgaattacaaccacgtccatttccacgaccagaggagcatgaaggagaacagtggaaaggaaggcatcgtcatgaagacccggaagagagggcgagaatgagactgagggaagcagaaagaatagagagggaaagggaggaacagagggtgagggaggaggaagagaaactccggcgacgtgagaagaaggaacaacgggaagagaaagaagaagaaggtcaaggttcggaagactcacactcaaaaagacagaacaacccttttcacttcagctctaacaggttccaaacgctcttcaagaaccaacatggtcaccttcgcgtcctccagagattcgaccaacgctccccacaacttgagaaccttcgagactaccgtgttgtagagctcatggctaaacccaacaccctctttctcccccaccacgctgacgctgattttctcctccttgtccttagcggttagtacacttaactaattactactactaattaaacatcgatcatcactttgcttctgctcttagtactagttaatggtaatcaattttactaattacagggagagccttaattaacttggtgaaacccgacgacagagacccctactaccttgaccgtggttatgcacaaagaatccctgcaggaaccactgtctatttggttaacccggacaagaagaaagatctcagagtgattaaactcgccatacccgttaacaaacctggcaattttgaggtactgcatgcctttttcattatgacagattagttaattaattcttctaattaatttatttccaaatgtgaagtgaatatcaatctttgtccttttctctttctctaggatttcttcctatctagcactcaagaccagcaatcctacttgcaaggcttcagcgagaatattctggaggcttccttcaatgtaagcaagcaacacatgcaccatctaatttgcatttccgtttagtggtttgtctaaatgtcatactaattaaatattatcattatatttgcttgtgtgcgcaattggttttatagaccaaattcgaggagataaacagggttttgtttggtggagaggggaggcggcaccagcaagagggagtgattttggaactatcaaaggaacagattcgggaactgagcaagcgtgccaaatctagttcaaggagtactaattccttcgattatgaaccattctacttgagaggctcccaaatctcttccaacaactttggaaagttctatgagatcacccctgagaagaacccccagcttcgggactttgatatcctcctcaatactgtggatatcaacgaggtaagtgcaaaaaccatgtagatctagacaaagtgaactgcactattgagttctattcactctcttcttaattagttagttatactaattattgagactaaacaaatacagggaggtcttcttctgccacactacaattcaaaggccatagtgatactaatggttactgaaggagaagcaaacattgaactcgttggcctaaaagaacagcaacagggagaggaaacaagggaagtgcgaaagtatagagccgagttgtctgaagacgatatatttgtaatcccagcagcttatccatttgtggtcaacgctacctcaaacctcaatttcgttgcttttggtatcaatgctgagaacaaccagaggaacttccttgcaggtactatacatatataatattgattattgaataccctgttgttactttttgttgttttatggcatgtggaagcatgctgattgggttatttaattatttccaattcttggtgcgtgtgattgaaaatttgaaggttcgaaagacaatgtgataagacagatacaaaaacaggtgaaggagcttgcgttccctgctgggtctgcacaagatatcgagaatctaataaagaaccagagggaatcctattttgctgatgctcagcctttgcaaaaggaggaagggaagaaagggtcctttgtcttttga

SEQ ID NO: 71 - DNA, 1791 bp, Glycine max
  source 1..1791: mol_type = other DNA
atgatagcgcggtttcctttgttgccgttgctgttgctgggaattgtttttctagcatcagtttctgtctcattacaggtgaagccgccgagtcaccacgagtgcctccagagttgcgagagtgaaacggatccatacaagcggaaagcatgcaagcttagttgcaagttcgttcctgagcacggggagcaacaacatgaagaagaagaaagagaaagagaacatccacaaccacacccaccacacgaggagagaggacgtagagaaagaggacgccaggaaggtgagaaggaggaaaaggaacaagacgagcgtgaattacaaccacgtccatttccacgaccagaggagcatgaaggagaacagtggaaaggaaggcatcgtcatgaagacccggaagagagggcgagaatgagactgagggaagcagaaagaatagagagggaaagggaggaacagagggtgagggaggaggaagagaaactccggcgacgtgagaagaaggaacaacgggaagagaaagaagaagaaggtcaaggttcggaagactcacactcaaaaagacagaacaacccttttcacttcagctctaacaggttccaaacgctcttcaagaaccaacatggtcaccttcgcgtcctccagagattcgaccaacgctccccacaacttgagaaccttcgagactaccgtgttgtagagctcatggctaaacccaacaccctctttctcccccaccacgctgacgctgattttctcctccttgtccttagcgggagagccttaattaacttggtgaaacccgacgacagagacccctactaccttgaccgtggttatgcacaaagaatccctgcaggaaccactgtctatttggttaacccggacaagaagaaagatctcagagtgattaaactcgccatacccgttaacaaacctggcaattttgaggatttcttcctatctagcactcaagaccagcaatcctacttgcaaggcttcagcgagaatattctggaggcttccttcaataccaaattcgaggagataaacagggttttgtttggtggagaggggaggcggcaccagcaagagggagtgattttggaactatcaaaggaacagattcgggaactgagcaagcgtgccaaatctagttcaaggagtactaattccttcgattatgaaccattctacttgagaggctcccaaatctcttccaacaactttggaaagttctatgagatcacccctgagaagaacccccagcttcgggactttgatatcctcctcaatactgtggatatcaacgagttagttatactaattattgagactaaacaaatacagggaggtcttcttctgccacactacaattcaaaggccatagtgatactaatggttactgaaggagaagcaaacattgaactcgttggcctaaaagaacagcaacagggagaggaaacaagggaagtgcgaaagtatagagccgagttgtctgaagacgatatatttgtaatcccagcagcttatccatttgtggtcaacgctacctcaaacctcaatttcgttgcttttggtatcaatgctgagaacaaccagaggaacttccttgcaggttcgaaagacaatgtgataagacagatacaaaaacaggtgaaggagcttgcgttccctgctgggtctgcacaagatatcgagaatctaataaagaaccagagggaatcctattttgctgatgctcagcctttgcaaaaggaggaagggaagaaagggtcctttgtcttttga

(19) United States
(12) Patent Application Publication
(10) Pub. No.: US 2023/0151378 A1
(43) Pub. Date: May 18, 2023

(54) Soybean Lines with High Seed Protein and Steady to High Oil Content

(71) Applicant: Board of Trustees of Southern Illinois University, Carbondale, IL (US)

(72) Inventors: Khalid Meksem, Carbondale, IL (US); Naoufal Lakhssassi, Carbondale, IL (US)

(73) Assignee: Board of Trustees of Southern Illinois University, Carbondale, IL (US)

(21) Appl. No.: 18/050,658

(22) Filed: Oct. 28, 2022

Related U.S. Application Data
(60) Provisional application No. 63/273,028, filed on Oct. 28, 2021.

(51) Int. Cl. C12N 15/82

(52) U.S. Cl. CPC C12N 15/8234; C12N 15/8251; C12N 15/8247

(57) ABSTRACT

The present invention is directed to a transgenic soybean plant having increased seed protein content and/or increased seed oil content comprising a polynucleotide encoding a β-ConGlycinin soybean seed storage promoter that functions in the soybean plant operably linked to a polynucleotide encoding a soybean seed storage polypeptide having β-ConGlycinin activity. The invention is further directed to a method of increasing seed protein content and/or increasing seed oil content of a soybean plant comprising transforming the soybean plant with a polynucleotide encoding a β-ConGlycinin soybean seed storage promoter that functions in the soybean plant operably linked to a polynucleotide encoding a soybean seed storage polypeptide having β-ConGlycinin activity.

### CROSS-REFERENCE TO RELATED APPLICATIONS

This application claims the benefit of U.S. Provisional Application No. 63/273,028, filed Oct. 28, 2021, the contents of which are incorporated herein in their entirety.

### INCORPORATION OF SEQUENCE LISTING XML

A computer readable form of the Sequence Listing XML containing the file named “3512490.004501 Sequence Listing.xml,” which is 117,534 bytes in size (as measured in MICROSOFT WINDOWS® EXPLORER) and was created on Oct. 26, 2022, is provided herein and is herein incorporated by reference. This Sequence Listing consists of SEQ ID NOs: 1-71.

### FIELD OF THE INVENTION

The present invention generally relates to compositions and methods to increase protein and oil content in soybeans.

### BACKGROUND OF THE INVENTION

Soybean production and the marketplace for soybeans is heavily driven by the value of protein in poultry and livestock feeding. Soyfood products like tofu and soymilk have increasingly became the popular choice in the vegetarian diet due to nutritional value and health benefits of their plant-based protein source. Nevertheless, the negative correlation between seed protein and oil content and between protein content and yield has severely hampered the development of high-yield soybean cultivars with elevated protein and oil content, for example, where a 1% reduction in total oil content may lead to a 2% increase in total protein content (FIG. 1). Although the relationship (i.e. negative correlation) between protein content and yield could be alleviated through the multiple crossing strategy, the development of high-protein and high-yield soybean lines is still a key desire of the field. A concerted effort has been made to better understand the mechanisms controlling protein content in soybean seed and underline the essence of protein source in the soybean industry.

Several QTLs controlling protein content in soybean were previously reported. In a population developed from a cross between a G. max line A81-356022 and a G. soja accession PI468916, two major QTLs on chromosomes 20 and 15, controlling seed protein content, have been identified using restriction fragment length polymorphism (RFLP) markers. These two QTLs for protein content were then given confirmed designations as cqPRO-003 (chr 20 QTL) and cqPRO-001 (chr 15 QTL) (http://soybase.org/). Localization of the QTL on chromosome 20 were fine mapped to a 3 centimorgan (cM) interval between Satt239 and ACG9b markers. Another QTL on chromosome 15 from the high protein line PI407788A was identified and mapped in a 535 kb interval. Furthermore, due to soybean genome duplication events, it has been revealed that unique genes within the duplicated genomic regions might also contribute to seed protein content. The soybean β-conglycinin gene family has been characterized with at least 15 members, due to duplication, structural variations, and the fact that their gene expression were under transcriptional and posttranscriptional regulations. The evolution of two multi-subunit seed storage protein gene families in soybean, glycinin and β-conglycinin, have been further studied to disclose the gain and loss of function of duplicated genes. Multiple QTLs have been mapped to control glycinin and β-conglycinin content in soybean seed storage protein, including loci containing CG4 and Gy4. The soybean plant introduction line, PI605781 B, containing natural spontaneous mutations in both Gy4 and Gy1, has been discovered to exhibit reduced glycinin content but an unchanged total seed protein content. It was previously unclear if mutations on the β-conglycinin result in unchanged protein content or have a positive impact on the protein content in soybean. Recently, a deletion on chromosome 12 was associated with elevated protein content using a fast neutron (FN) induced soybean mutant. To improve the quality of soybean protein, a recent study has been conducted to identify QTLs on chromosomes 1, 6, 8, 9, 10, 17, and 20 associated with levels of essential amino acids.

From the genomic scale, 40 SNPs in 17 different genomic regions have been identified to associate with seed protein content in a genome-wide association study (GWAS), including previously reported QTL controlling seed protein content in soybean. Mutants with a protein content range of 35.73-49.31% have been discovered from a large-scale soybean fast neutron mutant population. However, chromosomal large deletions related to fast neutron mutagenesis usually impact soybean agronomic performance. A study of mechanism of QQS (Qua-Quine Starch; At3g30720) modulating carbon and nitrogen partitioning exploited the potential to develop a nontransgenic high protein soybean line while maintaining oil content and yield.

Accordingly, there remains a need in the art to develop improved methods to increase protein and oil content in soybeans. There also remains a need in the art for development of transgenic soybean plants having increased seed protein content and/or increased seed oil content as compared to non-modified soybean plants.

### BRIEF SUMMARY OF THE INVENTION

One aspect of the present invention is directed to a transgenic soybean plant having increased seed protein content and/or increased seed oil content. The transgenic soybean plant comprises a polynucleotide encoding a β-ConGlycinin soybean seed storage promoter that functions in the soybean plant operably linked to a polynucleotide encoding a soybean seed storage polypeptide having β-ConGlycinin activity.

Another aspect of the present invention is directed to an agronomically elite soybean variety with increased seed protein content and/or increased seed oil content. The agronomically elite soybean variety comprises a polynucleotide encoding a β-ConGlycinin soybean seed storage promoter that functions in the soybean plant operably linked to a polynucleotide encoding a soybean seed storage polypeptide having β-ConGlycinin activity.

A further aspect of the present invention is directed to a DNA construct comprising a polynucleotide encoding a β-ConGlycinin soybean seed storage promoter that functions in the soybean plant operably linked to a polynucleotide encoding a soybean seed storage polypeptide having β-ConGlycinin activity.

A still further aspect of the present invention is directed to a method of increasing seed protein content and/or increasing seed oil content of a soybean plant. The method comprises transforming the soybean plant with a polynucleotide encoding a β-ConGlycinin soybean seed storage promoter that functions in the soybean plant operably linked to a polynucleotide encoding a soybean seed storage polypeptide having β-ConGlycinin activity.

Other objects and features will be in part apparent and in part pointed out hereinafter.

### DESCRIPTION OF THE DRAWINGS

The patent or application file contains at least one drawing executed in color. Copies of this patent or patent application publication with color drawing(s) will be provided by the Office upon request and payment of the necessary fee.

The following drawings form part of the present specification and are included to further demonstrate certain aspects of the present invention. The present invention may be better understood by reference to one or more of these drawings in combination with the detailed description of specific embodiments presented herein. However, those of skill in the art will understand that the drawings, described below, are for illustrative purposes only. The drawings are not intended to limit the scope of the present teachings in any way.

FIG. 1 is directed to a schematic illustrating the correlations among different seed components and the yield in soybean. Positive correlation is represented by (+) and negative correlation is represented by (−).

FIG. 2 is directed to a chart showing the protein sequence alignment of the β-ConGlycinin members from different plant sequenced species (SEQ ID NOs: 1-50).

FIG. 3 is directed to a diagram depicting the phylogenetic tree of the β-Conglycinin subunits from 21 plant species including six model plants; A. trichopoda (angiosperm), S. moellendorfii (lycophyte), M truncatula (leguminous), O. sativa (monocots), Z. mays (monocots), and A. thaliana (dicots), in addition to G. max (soybean) highlighted.

FIG. 4 is directed to a table showing the Ggene ID and expression of each subunit of the soybean β-conglycinin genes.

FIG. 5 is directed to a chart showing the physical positions corresponding to the nine Conglicinins on the four chromosomes carrying the genome of Soybean (Glicine Max). Glyma.10g246300 (Chr10:47,483,367); Glyma.10g028300 (Chr10:2,452,488); Glyma.02g145700 (Chr02:15,053,070); Glyma.10g246500 (Chr10:47,495,824); Glyma.20g148200 (Chr20:38,664,718); Glyma.20g146200 (Chr20:38,469,449); Glyma.20g148300 (Chr20:38,678,525); Glyma.20g148400 (Chr20:38,687,127); Glyma.13G328400 (Chr13:42,306,016).

FIGS. 6(a)-6(e) is directed to a series of schematic representations of the nine Conglycinin family members containing duplicated segments identified in the soybean genome. FIG. 6(a) reports a summary of segment and tandem duplications of the β-conglycinin gene family. FIG. 6(b) illustrates that CoGy2 and CoGy3 belong to a duplicated segment of 249 conserved genes between Chr2 and Chr10. FIG. 6(c) illustrates that CoGy9 and Glyma.15G045300 in chr13 and chr15 belongs to a duplicated segment with 759 conserved genes or anchors. FIG. 6(d) illustrates that the presence of possible tandem duplication between CoGy5, CoGy7, and CoGy8 in Chr20. FIG. 6(e) illustrates that the presence of other possible tandem duplications between CoGy6 and Glyma.20g146300 in Chr20, and between CoGy1 and CoGy4 in Chr10.

FIG. 7 is directed to a table showing the seed meal phenotypes of the selected mutant lines (M3 and M4 generations) and ‘Forrest’ wild-type.

FIG. 8 is directed to a table showing the designed probes used for TILLING by Target Capture Sequencing to target the four highly expressed ConGlycinin genes in soybean seeds.

FIG. 9 is directed to a table showing SNP mutations, indels, and mutation density for the GmCoGy1, GmCoGy2, GmCoGy3, and GmCoGy4 genes.

FIGS. 10A and 10B are directed to bar graphs illustrating the expression pattern of the nine soybean β-ConGlycinin CoGy, Glyma.20g146300, and Glyma.15g045300 genes in planta based on Soybase resource available from RNAsequencing data (http://www.soybase.org/soyseq). CoGy1 Glyma10g39150), CoGy2 (Glyma10g03390), CoGy3 (Glyma02g16440), and CoGy4 (Glyma10g39170) encode the seed specific isoforms in both Williams 82 (FIG. 10A) and Forrest (FIG. 10B) cultivars.

FIG. 11 is directed to a table showing the list of selected mutations in the conglycinin genes identified by TbyS⁺ and their corresponding protein and oil content.

FIG. 12 is directed to a three dimensional drawing showing the identification of missense β-ConGlycinin CoGy1 (Glyma.10g246300) mutants using TILLING by Target Capture Sequencing. Homology modeling of the β-ConGlycinin CoGy1 protein from Williams 82 is also illustrated, with important mutated CoGy1_{R231C}, CoGy1_{R247*}, and CoGy1_{R510Q} residue.

FIG. 13 is directed to a three dimensional drawing showing the identification of missense β-ConGlycinin CoGy2 (Glyma.10g028300) mutants using TILLING by Target Capture Sequencing. Homology modeling of the β-ConGlycinin CoGy2 protein from Williams 82 with important mutated CoGy2_{S255N} and CoGy2_{R287K} residues.

FIG. 14 is directed to a three dimensional drawing showing the identification of missense β-ConGlycinin CoGy4 (Glyma.10g246500) mutants using TILLING by Target Capture Sequencing. Homology modeling of the β-ConGlycinin CoGy4 protein from Williams 82 with important mutated CoGy4_{D249D}, CoGy4_{D296N}, CoGy4_{K461K}, and CoGy4_{T368M} residues.

FIG. 15 is directed to a three dimensional drawing showing the identification of missense β-ConGlycinin GmCoGy3 (Glyma.02g145700) mutants using TILLING by Target Capture Sequencing. Homology modeling of the β-ConGlycinin CoGy3 protein from Williams 82 with mutated GmCoGy3K224K, GmCoGy3L225F, and GmCoGy3S368L residues. The three different subunits constituting the β-ConGlycinin GmCoGy3 trimer are highlighted.

FIGS. 16A, 16B, 16C and 16D are directed to a series of photographs showing the soybean seed crude extract (SE) separated by two-dimensional electrophoresis (2D-PAGE). Proteins of the extract were separated using a pH gradient from 3.0 to 10.0 in the first dimension and 12.5% (w/v) gels in the second polyacrylamide gel. 2D-PAGE gels were stained with Coomassie Blue G250 for 4 min then distaining for 30 min. FIG. 16A is directed to Forrest-WT, FIG. 16B is directed to a CoGy1 mutant, FIG. 16C is directed to a CoGy2 mutant, and FIG. 16D is directed to a CoGy4 mutant. White circles show the newly detected proteins on the CoGy TILLING mutants when compared to the Forrest-WT.

FIG. 17 is a table showing the amino acid composition of the conglycinin mutants identified by TbyS⁺.

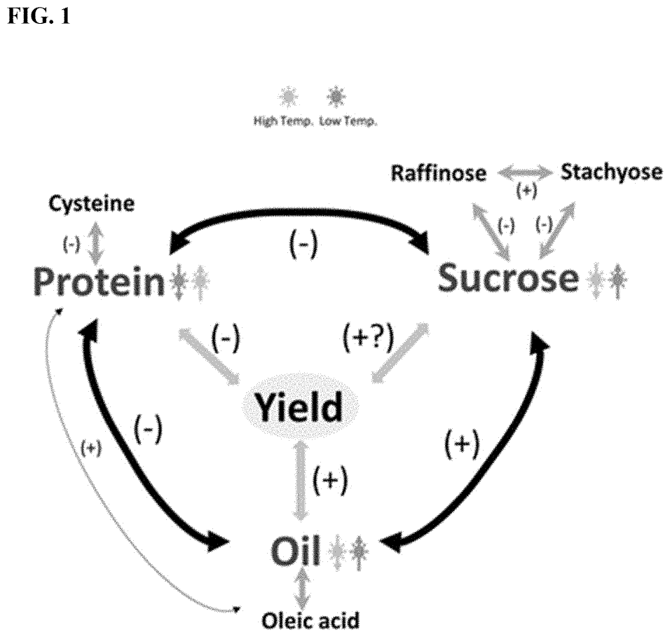

### DETAILED DESCRIPTION OF THE INVENTION

Transgenic Soybean Plants

One embodiment of the present invention is directed to a transgenic soybean plant with increased seed protein content and/or increased seed oil content comprising a polynucleotide encoding a soybean seed storage related promoter that functions in the soybean plant operably linked to a polynucleotide encoding a soybean seed storage polypeptide.

The polynucleotide encoding a soybean seed storage related promoter may comprise any wild type soybean seed storage promoter sequence, or a sequence at least 95% identical thereto, or a full length complement thereof, or a functional fragment thereof.

The soybean seed storage polypeptide may comprise any wild type soybean seed storage sequence, or a sequence at least 95% identical thereto, or a full-length complement thereof, or a functional fragment thereof.

The polynucleotide encoding a soybean seed storage polypeptide may comprise any wild type soybean seed storage genomic or coding sequence, or a sequence at least 95% identical thereto, or a full-length complement thereof, or a functional fragment thereof.

The polynucleotide encoding a soybean seed storage related promoter may comprise any wild type β-Conglycinin CoGy1 promoter sequence, or a sequence at least 95% identical thereto, or a full length complement thereof, or a functional fragment thereof. The soybean seed storage polynucleotide may comprise the wild type “Williams 82” β-Conglycinin CoGy1 promoter sequence (SEQ ID NO: 56), or a sequence at least 95% identical thereto, or a full-length complement thereof, or a functional fragment thereof.

The soybean seed storage polypeptide may comprise the wild type “Forrest” β-Conglycinin CoGy1 sequence, or a sequence at least 95% identical thereto, or a full-length complement thereof, or a functional fragment thereof, and may further comprise one or more mutations of the wild type “Forrest” β-Conglycinin CoGy1 sequence selected from the group consisting of: A4T, W28*, R231C, R247*, and R510Q. The soybean seed storage polypeptide may comprise the wild type “Williams 82” β-Conglycinin CoGy1 sequence (SEQ ID NO: 57), or a sequence at least 95% identical thereto, or a full-length complement thereof, or a functional fragment thereof, and may further comprise one or more mutations of the wild type “Williams 82” 3-Conglycinin CoGy1 sequence selected from the group consisting of: A4T, W28*, R231C, R247*, and R510Q.

The polynucleotide encoding a soybean seed storage related polypeptide may comprise any wild type β-Conglycinin CoGy1 genomic or coding sequence, or a sequence at least 95% identical thereto, or a full length complement thereof, or a functional fragment thereof. The soybean seed storage polynucleotide may comprise the wild type “Williams 82” β-Conglycinin CoGy1 genomic (SEQ ID NO: 58) or coding (SEQ ID NO: 59) sequence, or a sequence at least 95% identical thereto, or a full-length complement thereof, or a functional fragment thereof, and may further comprise one or more mutations of the wild type “Williams 82” β-Conglycinin CoGy1 genomic sequence (SEQ ID NO: 58) selected from the group consisting of: G10A, G84A, C691T, C739T, and G1529A.

The polynucleotide encoding a soybean seed storage related promoter may comprise any wild type β-Conglycinin CoGy2 promoter sequence, or a sequence at least 95% identical thereto, or a full length complement thereof, or a functional fragment thereof. The soybean seed storage polynucleotide may comprise the wild type “Williams 82” β-Conglycinin CoGy2 promoter sequence (SEQ ID NO: 60), or a sequence at least 95% identical thereto, or a full-length complement thereof, or a functional fragment thereof.

In certain embodiment, the soybean seed storage polypeptide comprises the wild type “Forrest” β-Conglycinin CoGy2 sequence, or a sequence at least 95% identical thereto, or a full-length complement thereof, or a functional fragment thereof, and may further comprise one or more mutations of the wild type “Forrest” β-Conglycinin CoGy2 sequence selected from the group consisting of: S255N and R287K. The soybean seed storage polypeptide may comprise the wild type “Williams 82” β-Conglycinin CoGy2 sequence (SEQ ID NO: 61), or a sequence at least 95% identical thereto, or a full-length complement thereof, or a functional fragment thereof, and may further comprise one or more mutations of the wild type “Williams 82” β-Conglycinin CoGy2 sequence selected from the group consisting of: S255N and R287K.

In some embodiments, the polynucleotide encoding a soybean seed storage related polypeptide comprises any wild type β-Conglycinin CoGy2 genomic or coding sequence, or a sequence at least 95% identical thereto, or a full length complement thereof, or a functional fragment thereof. In various embodiment, the soybean seed storage polynucleotide comprises the wild type “Williams 82” β-Conglycinin CoGy2 genomic (SEQ ID NO: 62) or coding (SEQ ID NO: 63) sequence, or a sequence at least 95% identical thereto, or a full-length complement thereof, or a functional fragment thereof, and may further comprise one or more mutations of the wild type “Williams 82” β-Conglycinin CoGy2 genomic sequence (SEQ ID NO: 62) selected from the group consisting of: G764A and G860A.

In other embodiment, the polynucleotide encoding a soybean seed storage related promoter comprises any wild type β-Conglycinin CoGy3 promoter sequence, or a sequence at least 95% identical thereto, or a full length complement thereof, or a functional fragment thereof. In certain embodiments, the soybean seed storage polynucleotide comprises the wild type “Williams 82” β-Conglycinin CoGy3 promoter sequence (SEQ ID NO: 64), or a sequence at least 95% identical thereto, or a full-length complement thereof, or a functional fragment thereof.

In one embodiment, the soybean seed storage polypeptide comprises the wild type “Forrest” β-Conglycinin CoGy3 sequence, or a sequence at least 95% identical thereto, or a full-length complement thereof, or a functional fragment thereof, and may further comprise one or more mutations of the wild type “Forrest” β-Conglycinin CoGy3 sequence. in some embodiments, the soybean seed storage polypeptide comprises the wild type “Williams 82” β-Conglycinin CoGy3 sequence (SEQ ID NO: 65), or a sequence at least 95% identical thereto, or a full-length complement thereof, or a functional fragment thereof, and may further comprise one or more mutations of the wild type “Williams 82” β-Conglycinin CoGy3 sequence.

In various embodiments, the polynucleotide encoding a soybean seed storage related polypeptide comprises any wild type β-Conglycinin CoGy3 genomic or coding sequence, or a sequence at least 95% identical thereto, or a full length complement thereof, or a functional fragment thereof. In other embodiments, the soybean seed storage polynucleotide comprises the wild type “Williams 82” β-Conglycinin CoGy3 genomic (SEQ ID NO: 66) or coding (SEQ ID NO: 67) sequence, or a sequence at least 95% identical thereto, or a full-length complement thereof, or a functional fragment thereof, and may further comprise one or more mutations of the wild type “Williams 82” β-Conglycinin CoGy3 genomic sequence (SEQ ID NO: 66).

In certain embodiments, the polynucleotide encoding a soybean seed storage related promoter comprises any wild type β-Conglycinin CoGy4 promoter sequence, or a sequence at least 95% identical thereto, or a full length complement thereof, or a functional fragment thereof. In one embodiment, the soybean seed storage polynucleotide comprises the wild type “Williams 82” β-Conglycinin CoGy4 promoter sequence (SEQ ID NO: 68), or a sequence at least 95% identical thereto, or a full-length complement thereof, or a functional fragment thereof.

In some embodiments, the soybean seed storage polypeptide comprises the wild type “Forrest” β-Conglycinin CoGy4 sequence, or a sequence at least 95% identical thereto, or a full-length complement thereof, or a functional fragment thereof, and may further comprise one or more mutations of the wild type “Forrest” Conglycinin α′-Subunit β-Conglycinin CoGy4 sequence selected from the group consisting of: C39Y, D249D, D296N, and K461K. In other embodiments, the soybean seed storage polypeptide comprises the wild type “Williams 82” β-Conglycinin CoGy4 sequence (SEQ ID NO: 69), or a sequence at least 95% identical thereto, or a full-length complement thereof, or a functional fragment thereof, and may further comprise one or more mutations of the wild type “Williams 82” β-Conglycinin CoGy4 sequence selected from the group consisting of: C39Y, D249D, D296N, and K461K.

In one embodiment, the polynucleotide encoding a soybean seed storage related polypeptide comprised any wild type β-Conglycinin CoGy4 genomic or coding sequence, or a sequence at least 95% identical thereto, or a full length complement thereof, or a functional fragment thereof. In certain embodiments, the soybean seed storage polynucleotide comprises the wild type “Williams 82” β-Conglycinin CoGy4 genomic (SEQ ID NO: 70) or coding (SEQ ID NO: 71) sequence, or a sequence at least 95% identical thereto, or a full-length complement thereof, or a functional fragment thereof, and may further comprise one or more mutations of the wild type “Williams 82” β-Conglycinin CoGy4 genomic sequence (SEQ ID NO: 70) selected from the group consisting of: G116A, C747T, G886A, and G1383A.

In other embodiments, the transgenic soybean plant with increased seed protein content and/or increased seed oil content comprises more than one polynucleotide encoding a soybean seed storage related promoter that functions in the soybean plant, provided that each polynucleotide encoding a soybean seed storage related promoter that function in the soybean plant is operably linked to a polynucleotide encoding a soybean seed storage polypeptide.

In some embodiments, the more than one polynucleotide encoding a soybean seed storage related promoter may be selected from the group consisting of: (i) any wild type β-Conglycinin CoGy1 promoter sequence, or a sequence at least 95% identical thereto, or a full length complement thereof, or a functional fragment thereof, wherein the soybean seed storage polypeptide comprises the wild type “Forrest” β-Conglycinin CoGy1 sequence or the wild type “Williams 82” β-Conglycinin CoGy1 sequence (SEQ ID NO: 57), or a sequence at least 95% identical thereto, or a full-length complement thereof, or a functional fragment thereof, and further comprises one or more mutations of the wild type “Forrest” β-Conglycinin CoGy1 sequence or the wild type “Williams 82” β-Conglycinin CoGy1 sequence (SEQ ID NO: 57) selected from the group consisting of: A4T, W28*, R231C, R247*, and R510Q; (ii) any wild type β-Conglycinin CoGy2 promoter sequence, or a sequence at least 95% identical thereto, or a full length complement thereof, or a functional fragment thereof, wherein the soybean seed storage polypeptide comprises the wild type “Forrest” β-Conglycinin CoGy2 sequence or the wild type “Williams 82” β-Conglycinin CoGy2 sequence (SEQ ID NO: 61), or a sequence at least 95% identical thereto, or a full-length complement thereof, or a functional fragment thereof, and further comprises one or more mutations of the wild type “Forrest” β-Conglycinin CoGy2 sequence or the wild type “Williams 82” β-Conglycinin CoGy2 sequence (SEQ ID NO: 61) selected from the group consisting of: S255N and R287K; (iii) any wild type β-Conglycinin CoGy3 promoter sequence, or a sequence at least 95% identical thereto, or a full length complement thereof, or a functional fragment thereof, wherein the soybean seed storage polypeptide comprises the wild type “Forrest” 3-Conglycinin CoGy3 sequence or the wild type “Williams 82” β-Conglycinin CoGy3 sequence (SEQ ID NO: 65), or a sequence at least 95% identical thereto, or a full-length complement thereof, or a functional fragment thereof, and further comprises one or more mutations of the wild type “Forrest” β-Conglycinin CoGy3 sequence or the wild type “Williams 82” β-Conglycinin CoGy3 sequence (SEQ ID NO: 65); and (iv) any wild type Conglycinin CoGy4 promoter sequence, or a sequence at least 95% identical thereto, or a full length complement thereof, or a functional fragment thereof, wherein the soybean seed storage polypeptide comprises the wild type “Forrest” Conglycinin CoGy4 sequence or the wild type “Williams 82” β-Conglycinin CoGy4 sequence (SEQ ID NO: 69), or a sequence at least 95% identical thereto, or a full-length complement thereof, or a functional fragment thereof, and further comprises one or more mutations of the wild type “Forrest” Conglycinin CoGy4 sequence or the wild type “Williams 82” β-Conglycinin CoGy4 sequence (SEQ ID NO: 69) selected from the group consisting of: C39Y, D249D, D296N, and K461K.

In one embodiment, the transgenic soybean plant may have increased seed protein content as compared to a control soybean plant lacking the polynucleotide encoding a soybean seed storage polynucleotide as described herein.

In another embodiment, the transgenic soybean plant may have increased seed protein content as compared to a control soybean plant lacking the polynucleotide encoding a soybean seed storage polynucleotide comprising one or more mutation as described herein.

In some embodiments, the transgenic soybean plant may have increased seed oil content as compared to a control soybean plant lacking the polynucleotide encoding a soybean seed storage polynucleotide as described herein.

In other embodiments, the transgenic soybean plant may have increased seed oil content as compared to a control soybean plant lacking the polynucleotide encoding a soybean seed storage polynucleotide comprising one or more mutation as described herein.

In still further embodiment, the transgenic soybean plant may have both increased seed protein content as compared to a control soybean plant lacking the polynucleotide encoding a soybean seed storage polynucleotide as described above and increased seed oil content as compared to a control soybean plant lacking the polynucleotide encoding a soybean seed storage polynucleotide as described herein.

In another embodiment, the transgenic soybean plant may have both increased seed protein content as compared to a control soybean plant lacking the polynucleotide encoding a soybean seed storage polynucleotide as described above and increased seed oil content as compared to a control soybean plant lacking the polynucleotide encoding a soybean seed storage polynucleotide comprising one or more mutation as described herein.

In various embodiment, the increased seed protein content of the plant of the present invention represents an at least about 1%, at least about 2%, at least about 3%, at least about 4%, at least about 5%, at least about 6%, at least about 7%, at least about 8%, at least about 9%, at least about 10%, at least about 20%, at least about 30%, at least about 40%, at least about 50%, at least about 60%, at least about 70%, at least about 80%, at least about 90%, or at least about 100% increase in seed protein content as compared to the control soybean plant lacking the polynucleotide encoding a soybean seed storage polynucleotide as described herein. In other embodiments, the increased seed protein content of the plant of the present invention represents an at least about 1%, at least about 2%, at least about 3%, at least about 4%, at least about 5%, at least about 6%, at least about 7%, at least about 8%, at least about 9%, at least about 10%, at least about 20%, at least about 30%, at least about 40%, at least about 50%, at least about 60%, at least about 70%, at least about 80%, at least about 90%, or at least about 100% increase in seed protein content as compared to the control soybean plant lacking the polynucleotide encoding a soybean seed storage polynucleotide comprising one or more mutation as described herein.

In some embodiments, the increased seed oil content of the plant of the present invention represents an at least about 1%, at least about 2%, at least about 3%, at least about 4%, at least about 5%, at least about 6%, at least about 7%, at least about 8%, at least about 9%, at least about 10%, at least about 20%, at least about 30%, at least about 40%, at least about 50%, at least about 60%, at least about 70%, at least about 80%, at least about 90%, or at least about 100% increase in seed oil content as compared to the control soybean plant lacking the polynucleotide encoding a soybean seed storage polynucleotide as described herein. In further embodiments, the increased seed oil content of the plant of the present invention represents an at least about 1%, at least about 2%, at least about 3%, at least about 4%, at least about 5%, at least about 6%, at least about 7%, at least about 8%, at least about 9%, at least about 10%, at least about 20%, at least about 30%, at least about 40%, at least about 50%, at least about 60%, at least about 70%, at least about 80%, at least about 90%, or at least about 100% increase in seed oil content as compared to the control soybean plant lacking the polynucleotide encoding a soybean seed storage polynucleotide comprising one or more mutation as described herein.

An additional embodiment of the disclosed technology is a plant part of any of the transgenic soybean plants described above.

Agronomically Elite Soybean Varieties

Another embodiment of the present invention is a plant of an agronomically elite soybean variety with increased seed protein content and/or increased seed oil content comprising a polynucleotide encoding a soybean seed storage related promoter that functions in the soybean plant operably linked to a polynucleotide encoding a soybean seed storage polypeptide.

The polynucleotide encoding a soybean seed storage related promoter may comprise any wild type soybean seed storage promoter sequence, or a sequence at least 95% identical thereto, or a full length complement thereof, or a functional fragment thereof.

The soybean seed storage polypeptide may comprise any wild type soybean seed storage sequence, or a sequence at least 95% identical thereto, or a full-length complement thereof, or a functional fragment thereof.

In certain embodiments, the present invention is directed to a plant of an agronomically elite soybean variety with increased seed protein content and/or increased seed oil content comprising a polynucleotide encoding a β-ConGlycinin soybean seed storage promoter that functions in the soybean plant operably linked to a polynucleotide encoding a soybean seed storage polypeptide having β-ConGlycinin activity.

The polynucleotide encoding a soybean seed storage related promoter may comprise any wild type β-Conglycinin CoGy1 promoter sequence, or a sequence at least 95% identical thereto, or a full length complement thereof, or a functional fragment thereof. The soybean seed storage polynucleotide may comprise the wild type “Williams 82” β-Conglycinin CoGy1 promoter sequence (SEQ ID NO: 56) or “Forrest” β-Conglycinin CoGy1 promoter sequence, or a sequence at least 95% identical thereto, or a full-length complement thereof, or a functional fragment thereof.

The soybean seed storage polypeptide may comprise the wild type “Forrest” β-Conglycinin CoGy1 sequence, or a sequence at least 95% identical thereto, or a full-length complement thereof, or a functional fragment thereof, and may further comprise one or more mutations of the wild type “Forrest” β-Conglycinin CoGy1 sequence selected from the group consisting of: A4T, W28*, R231C, R247*, and R510Q. The soybean seed storage polypeptide may comprise the wild type “Williams 82” β-Conglycinin CoGy1 sequence (SEQ ID NO: 57), or a sequence at least 95% identical thereto, or a full-length complement thereof, or a functional fragment thereof, and may further comprise one or more mutations of the wild type “Williams 82” 3-Conglycinin CoGy1 sequence selected from the group consisting of: A4T, W28*, R231C, R247*, and R510Q.

The polynucleotide encoding a soybean seed storage related polypeptide may comprise any wild type β-Conglycinin CoGy1 genomic or coding sequence, or a sequence at least 95% identical thereto, or a full length complement thereof, or a functional fragment thereof. The soybean seed storage polynucleotide may comprise the wild type “Williams 82” β-Conglycinin CoGy1 genomic (SEQ ID NO: 58) or coding (SEQ ID NO: 59) sequence, or a sequence at least 95% identical thereto, or a full-length complement thereof, or a functional fragment thereof, and may further comprise one or more mutations of the wild type “Williams 82” β-Conglycinin CoGy1 genomic sequence (SEQ ID NO: 58) selected from the group consisting of: G10A, G84A, C691T, C739T, and G1529A. The soybean seed storage polynucleotide may comprise the wild type “Forrest” β-Conglycinin CoGy1 genomic or coding sequence, or a sequence at least 95% identical thereto, or a full-length complement thereof, or a functional fragment thereof, and may further comprise one or more mutations of the wild type “Forrest” β-Conglycinin CoGy1 genomic sequence selected from the group consisting of: G10A, G84A, C691T, C739T, and G1529A.

The polynucleotide encoding a soybean seed storage related promoter may comprise any wild type β-Conglycinin CoGy2 promoter sequence, or a sequence at least 95% identical thereto, or a full length complement thereof, or a functional fragment thereof. The soybean seed storage polynucleotide may comprise the wild type “Williams 82” β-Conglycinin CoGy2 promoter sequence (SEQ ID NO: 60) or “Forrest” β-Conglycinin CoGy2 promoter sequence, or a sequence at least 95% identical thereto, or a full-length complement thereof, or a functional fragment thereof.

The soybean seed storage polypeptide may comprise the wild type “Forrest” β-Conglycinin CoGy2 sequence, or a sequence at least 95% identical thereto, or a full-length complement thereof, or a functional fragment thereof, and may further comprise one or more mutations of the wild type “Forrest” β-Conglycinin CoGy2 sequence selected from the group consisting of: S255N and R287K. The soybean seed storage polypeptide may comprise the wild type “Williams 82” β-Conglycinin CoGy2 sequence (SEQ ID NO: 61), or a sequence at least 95% identical thereto, or a full-length complement thereof, or a functional fragment thereof, and may further comprise one or more mutations of the wild type “Williams 82” β-Conglycinin CoGy2 sequence selected from the group consisting of: S255N and R287K.

The polynucleotide encoding a soybean seed storage related polypeptide may comprise any wild type β-Conglycinin CoGy2 genomic or coding sequence, or a sequence at least 95% identical thereto, or a full length complement thereof, or a functional fragment thereof. The soybean seed storage polynucleotide may comprise the wild type “Williams 82” β-Conglycinin CoGy2 genomic (SEQ ID NO: 62) or coding (SEQ ID NO: 63) sequence, or a sequence at least 95% identical thereto, or a full-length complement thereof, or a functional fragment thereof, and may further comprise one or more mutations of the wild type “Williams 82” β-Conglycinin CoGy2 genomic sequence (SEQ ID NO: 62) selected from the group consisting of: G764A and G860A.

The polynucleotide encoding a soybean seed storage related promoter may comprise any wild type β-Conglycinin CoGy3 promoter sequence, or a sequence at least 95% identical thereto, or a full length complement thereof, or a functional fragment thereof. The soybean seed storage polynucleotide may comprise the wild type “Williams 82” β-Conglycinin CoGy3 promoter sequence (SEQ ID NO: 64) or “Forrest” β-Conglycinin CoGy3 promoter sequence, or a sequence at least 95% identical thereto, or a full-length complement thereof, or a functional fragment thereof.

The soybean seed storage polypeptide may comprise the wild type “Forrest” β-Conglycinin CoGy3 sequence, or a sequence at least 95% identical thereto, or a full-length complement thereof, or a functional fragment thereof, and may further comprise one or more mutations of the wild type “Forrest” β-Conglycinin CoGy3 sequence. The soybean seed storage polypeptide may comprise the wild type “Williams 82” β-Conglycinin CoGy3 sequence (SEQ ID NO: 65), or a sequence at least 95% identical thereto, or a full-length complement thereof, or a functional fragment thereof, and may further comprise one or more mutations of the wild type “Williams 82” β-Conglycinin CoGy3 sequence.

The polynucleotide encoding a soybean seed storage related polypeptide may comprise any wild type β-Conglycinin CoGy3 genomic or coding sequence, or a sequence at least 95% identical thereto, or a full length complement thereof, or a functional fragment thereof. The soybean seed storage polynucleotide may comprise the wild type “Williams 82” β-Conglycinin CoGy3 genomic (SEQ ID NO: 66) or coding (SEQ ID NO: 67) sequence, or a sequence at least 95% identical thereto, or a full-length complement thereof, or a functional fragment thereof, and may further comprise one or more mutations of the wild type “Williams 82” β-Conglycinin CoGy3 genomic sequence (SEQ ID NO: 66).

The polynucleotide encoding a soybean seed storage related promoter may comprise any wild type β-Conglycinin CoGy4 promoter sequence, or a sequence at least 95% identical thereto, or a full length complement thereof, or a functional fragment thereof. The soybean seed storage polynucleotide may comprise the wild type “Williams 82” β-Conglycinin CoGy4 promoter sequence (SEQ ID NO: 68) or “Forrest” β-Conglycinin CoGy4 promoter sequence, or a sequence at least 95% identical thereto, or a full-length complement thereof, or a functional fragment thereof.

The soybean seed storage polypeptide may comprise the wild type “Forrest” β-Conglycinin CoGy4 sequence, or a sequence at least 95% identical thereto, or a full-length complement thereof, or a functional fragment thereof, and may further comprise one or more mutations of the wild type “Forrest” Conglycinin α′-Subunit β-Conglycinin CoGy4 sequence selected from the group consisting of: C39Y, D249D, D296N, and K461K. The soybean seed storage polypeptide may comprise the wild type “Williams 82” β-Conglycinin CoGy4 sequence (SEQ ID NO: 69), or a sequence at least 95% identical thereto, or a full-length complement thereof, or a functional fragment thereof, and may further comprise one or more mutations of the wild type “Williams 82” β-Conglycinin CoGy4 sequence selected from the group consisting of: C39Y, D249D, D296N, and K461K.

The polynucleotide encoding a soybean seed storage related polypeptide may comprise any wild type β-Conglycinin CoGy4 genomic or coding sequence, or a sequence at least 95% identical thereto, or a full length complement thereof, or a functional fragment thereof. The soybean seed storage polynucleotide may comprise the wild type “Williams 82” β-Conglycinin CoGy4 genomic (SEQ ID NO: 70) or coding (SEQ ID NO: 71) sequence, or a sequence at least 95% identical thereto, or a full-length complement thereof, or a functional fragment thereof, and may further comprise one or more mutations of the wild type “Williams 82” β-Conglycinin CoGy4 genomic sequence (SEQ ID NO: 70) selected from the group consisting of: G116A, C747T, G886A, and G1383A. The soybean seed storage polynucleotide may comprise the wild type “Forrest” β-Conglycinin CoGy4 genomic or coding sequence, or a sequence at least 95% identical thereto, or a full-length complement thereof, or a functional fragment thereof, and may further comprise one or more mutations of the wild type “Forrest” β-Conglycinin CoGy4 genomic sequence selected from the group consisting of: G116A, C747T, G886A, and G1383A.

The plant with increased seed protein content and/or increased seed oil content may comprise more than one polynucleotide encoding a soybean seed storage related promoter that functions in the soybean plant, provided that each polynucleotide encoding a soybean seed storage related promoter that function in the soybean plant is operably linked to a polynucleotide encoding a soybean seed storage polypeptide.

The more than one polynucleotide encoding a soybean seed storage related promoter may be selected from the group consisting of: (i) any wild type β-Conglycinin CoGy1 promoter sequence, or a sequence at least 95% identical thereto, or a full length complement thereof, or a functional fragment thereof, wherein the soybean seed storage polypeptide comprises the wild type “Forrest” β-Conglycinin CoGy1 sequence or the wild type “Williams 82” β-Conglycinin CoGy1 sequence (SEQ ID NO: 57), or a sequence at least 95% identical thereto, or a full-length complement thereof, or a functional fragment thereof, and further comprises one or more mutations of the wild type “Forrest” β-Conglycinin CoGy1 sequence or the wild type “Williams 82” β-Conglycinin CoGy1 sequence (SEQ ID NO: 57) selected from the group consisting of: A4T, W28*, R231C, R247*, and R510Q; (ii) any wild type β-Conglycinin CoGy2 promoter sequence, or a sequence at least 95% identical thereto, or a full length complement thereof, or a functional fragment thereof, wherein the soybean seed storage polypeptide comprises the wild type “Forrest” β-Conglycinin CoGy2 sequence or the wild type “Williams 82” β-Conglycinin CoGy2 sequence (SEQ ID NO: 61), or a sequence at least 95% identical thereto, or a full-length complement thereof, or a functional fragment thereof, and further comprises one or more mutations of the wild type “Forrest” β-Conglycinin CoGy2 sequence or the wild type “Williams 82” β-Conglycinin CoGy2 sequence (SEQ ID NO: 61) selected from the group consisting of: S255N and R287K; (iii) any wild type β-Conglycinin CoGy3 promoter sequence, or a sequence at least 95% identical thereto, or a full length complement thereof, or a functional fragment thereof, wherein the soybean seed storage polypeptide comprises the wild type “Forrest” 3-Conglycinin CoGy3 sequence or the wild type “Williams 82” β-Conglycinin CoGy3 sequence (SEQ ID NO: 65), or a sequence at least 95% identical thereto, or a full-length complement thereof, or a functional fragment thereof, and further comprises one or more mutations of the wild type “Forrest” β-Conglycinin CoGy3 sequence or the wild type “Forrest” β-Conglycinin CoGy3 sequence (SEQ ID NO: 65); and (iv) any wild type Conglycinin CoGy4 promoter sequence, or a sequence at least 95% identical thereto, or a full length complement thereof, or a functional fragment thereof, wherein the soybean seed storage polypeptide comprises the wild type “Forrest” Conglycinin CoGy4 sequence or the wild type “Williams 82” β-Conglycinin CoGy4 sequence (SEQ ID NO: 69), or a sequence at least 95% identical thereto, or a full-length complement thereof, or a functional fragment thereof, and further comprises one or more mutations of the wild type “Forrest” Conglycinin CoGy4 sequence or the wild type “Williams 82” β-Conglycinin CoGy4 sequence (SEQ ID NO: 69) selected from the group consisting of: C39Y, D249D, D296N, and K461K.

The plant may have increased seed protein content as compared to a control soybean plant lacking the polynucleotide encoding a soybean seed storage polynucleotide as described above.

The plant may have increased seed oil content as compared to a control soybean plant lacking the polynucleotide encoding a soybean seed storage polynucleotide as described above.

The plant may have both increased seed protein content as compared to a control soybean plant lacking the polynucleotide encoding a soybean seed storage polynucleotide as described above and increased seed oil content as compared to a control soybean plant lacking the polynucleotide encoding a soybean seed storage polynucleotide as described above.

The increased seed protein content may comprise at least about 1%, at least about 2%, at least about 3%, at least about 4%, at least about 5%, at least about 6%, at least about 7%, at least about 8%, at least about 9%, at least about 10%, at least about 20%, at least about 30%, at least about 40%, at least about 50%, at least about 60%, at least about 70%, at least about 80%, at least about 90%, or at least about 100% increase in seed protein content as compared to the control soybean plant lacking the polynucleotide encoding a soybean seed storage polynucleotide as described above.

The increased seed oil content may comprise at least about 1%, at least about 2%, at least about 3%, at least about 4%, at least about 5%, at least about 6%, at least about 7%, at least about 8%, at least about 9%, at least about 10%, at least about 20%, at least about 30%, at least about 40%, at least about 50%, at least about 60%, at least about 70%, at least about 80%, at least about 90%, or at least about 100% increase in seed oil content as compared to the control soybean plant lacking the polynucleotide encoding a soybean seed storage polynucleotide as described above.

An additional embodiment of the disclosed technology is a plant part of any of the plants described above.

Methods of Increasing Seed Protein Content and/or Increasing Seed Oil Content

Another embodiment of the present invention is a method of increasing seed protein content and/or increasing seed oil content of a soybean plant comprising transforming the soybean plant with a polynucleotide encoding a soybean seed storage related promoter that functions in the soybean plant operably linked to a polynucleotide encoding a soybean seed storage polypeptide.

The polynucleotide encoding a soybean seed storage related promoter may comprise any wild type soybean seed storage promoter sequence, or a sequence at least 95% identical thereto, or a full length complement thereof, or a functional fragment thereof.

The soybean seed storage polypeptide may comprise any wild type soybean seed storage sequence, or a sequence at least 95% identical thereto, or a full-length complement thereof, or a functional fragment thereof.

The polynucleotide encoding a soybean seed storage related promoter may comprise any wild type β-Conglycinin CoGy1 promoter sequence, or a sequence at least 95% identical thereto, or a full length complement thereof, or a functional fragment thereof. The soybean seed storage polynucleotide may comprise the wild type “Williams 82” β-Conglycinin CoGy1 promoter sequence (SEQ ID NO: 56), or a sequence at least 95% identical thereto, or a full-length complement thereof, or a functional fragment thereof.

The soybean seed storage polypeptide may comprise the wild type “Forrest” β-Conglycinin CoGy1 sequence, or a sequence at least 95% identical thereto, or a full-length complement thereof, or a functional fragment thereof, and may further comprise one or more mutations of the wild type “Forrest” β-Conglycinin CoGy1 sequence selected from the group consisting of: A4T, W28*, R231C, R247*, and R510Q. The soybean seed storage polypeptide may comprise the wild type “Williams 82” β-Conglycinin CoGy1 sequence (SEQ ID NO: 57), or a sequence at least 95% identical thereto, or a full-length complement thereof, or a functional fragment thereof, and may further comprise one or more mutations of the wild type “Williams 82” 3-Conglycinin CoGy1 sequence selected from the group consisting of: A4T, W28*, R231C, R247*, and R510Q.

The polynucleotide encoding a soybean seed storage related polypeptide may comprise any wild type β-Conglycinin CoGy1 genomic or coding sequence, or a sequence at least 95% identical thereto, or a full length complement thereof, or a functional fragment thereof. The soybean seed storage polynucleotide may comprise the wild type “Williams 82” β-Conglycinin CoGy1 genomic (SEQ ID NO: 58) or coding (SEQ ID NO: 59) sequence, or a sequence at least 95% identical thereto, or a full-length complement thereof, or a functional fragment thereof, and may further comprise one or more mutations of the wild type “Williams 82” β-Conglycinin CoGy1 genomic sequence (SEQ ID NO: 58) selected from the group consisting of: G10A, G84A, C691T, C739T, and G1529A.

The polynucleotide encoding a soybean seed storage related promoter may comprise any wild type β-Conglycinin CoGy2 promoter sequence, or a sequence at least 95% identical thereto, or a full length complement thereof, or a functional fragment thereof. The soybean seed storage polynucleotide may comprise the wild type “Williams 82” β-Conglycinin CoGy2 promoter sequence (SEQ ID NO: 60), or a sequence at least 95% identical thereto, or a full-length complement thereof, or a functional fragment thereof.

The soybean seed storage polypeptide may comprise the wild type “Forrest” β-Conglycinin CoGy2 sequence, or a sequence at least 95% identical thereto, or a full-length complement thereof, or a functional fragment thereof, and may further comprise one or more mutations of the wild type “Forrest” β-Conglycinin CoGy2 sequence selected from the group consisting of: S255N and R287K. The soybean seed storage polypeptide may comprise the wild type “Williams 82” β-Conglycinin CoGy2 sequence (SEQ ID NO: 61), or a sequence at least 95% identical thereto, or a full-length complement thereof, or a functional fragment thereof, and may further comprise one or more mutations of the wild type “Williams 82” β-Conglycinin CoGy2 sequence selected from the group consisting of: S255N and R287K.

The polynucleotide encoding a soybean seed storage related polypeptide may comprise any wild type β-Conglycinin CoGy2 genomic or coding sequence, or a sequence at least 95% identical thereto, or a full length complement thereof, or a functional fragment thereof. The soybean seed storage polynucleotide may comprise the wild type “Williams 82” β-Conglycinin CoGy2 genomic (SEQ ID NO: 62) or coding (SEQ ID NO: 63) sequence, or a sequence at least 95% identical thereto, or a full-length complement thereof, or a functional fragment thereof, and may further comprise one or more mutations of the wild type “Williams 82” β-Conglycinin CoGy2 genomic sequence (SEQ ID NO: 62) selected from the group consisting of: G764A and G860A.

The polynucleotide encoding a soybean seed storage related promoter may comprise any wild type β-Conglycinin CoGy3 promoter sequence, or a sequence at least 95% identical thereto, or a full length complement thereof, or a functional fragment thereof. The soybean seed storage polynucleotide may comprise the wild type “Williams 82” β-Conglycinin CoGy3 promoter sequence (SEQ ID NO: 64), or a sequence at least 95% identical thereto, or a full-length complement thereof, or a functional fragment thereof.

The soybean seed storage polypeptide may comprise the wild type “Forrest” β-Conglycinin CoGy3 sequence, or a sequence at least 95% identical thereto, or a full-length complement thereof, or a functional fragment thereof, and may further comprise one or more mutations of the wild type “Forrest” β-Conglycinin CoGy3 sequence. The soybean seed storage polypeptide may comprise the wild type “Williams 82” β-Conglycinin CoGy3 sequence (SEQ ID NO: 65), or a sequence at least 95% identical thereto, or a full-length complement thereof, or a functional fragment thereof, and may further comprise one or more mutations of the wild type “Williams 82” β-Conglycinin CoGy3 sequence.

The polynucleotide encoding a soybean seed storage related polypeptide may comprise any wild type β-Conglycinin CoGy3 genomic or coding sequence, or a sequence at least 95% identical thereto, or a full length complement thereof, or a functional fragment thereof. The soybean seed storage polynucleotide may comprise the wild type “Williams 82” β-Conglycinin CoGy3 genomic (SEQ ID NO: 66) or coding (SEQ ID NO: 67) sequence, or a sequence at least 95% identical thereto, or a full-length complement thereof, or a functional fragment thereof, and may further comprise one or more mutations of the wild type “Williams 82” β-Conglycinin CoGy3 genomic sequence (SEQ ID NO: 66).

The polynucleotide encoding a soybean seed storage related promoter may comprise any wild type β-Conglycinin CoGy4 promoter sequence, or a sequence at least 95% identical thereto, or a full length complement thereof, or a functional fragment thereof. The soybean seed storage polynucleotide may comprise the wild type “Williams 82” β-Conglycinin CoGy4 promoter sequence (SEQ ID NO: 68), or a sequence at least 95% identical thereto, or a full-length complement thereof, or a functional fragment thereof.

The soybean seed storage polypeptide may comprise the wild type “Forrest” (3-Conglycinin CoGy4 sequence, or a sequence at least 95% identical thereto, or a full-length complement thereof, or a functional fragment thereof, and may further comprise one or more mutations of the wild type “Forrest” Conglycinin α′-Subunit β-Conglycinin CoGy4 sequence selected from the group consisting of: C39Y, D249D, D296N, and K461K. The soybean seed storage polypeptide may comprise the wild type “Williams 82” β-Conglycinin CoGy4 sequence (SEQ ID NO: 69), or a sequence at least 95% identical thereto, or a full-length complement thereof, or a functional fragment thereof, and may further comprise one or more mutations of the wild type “Williams 82” β-Conglycinin CoGy4 sequence selected from the group consisting of: C39Y, D249D, D296N, and K461K.

The polynucleotide encoding a soybean seed storage related polypeptide may comprise any wild type β-Conglycinin CoGy4 genomic or coding sequence, or a sequence at least 95% identical thereto, or a full length complement thereof, or a functional fragment thereof. The soybean seed storage polynucleotide may comprise the wild type “Williams 82” β-Conglycinin CoGy4 genomic (SEQ ID NO: 70) or coding (SEQ ID NO: 71) sequence, or a sequence at least 95% identical thereto, or a full-length complement thereof, or a functional fragment thereof, and may further comprise one or more mutations of the wild type “Williams 82” β-Conglycinin CoGy4 genomic sequence (SEQ ID NO: 70) selected from the group consisting of: G116A, C747T, G886A, and G1383A.

The method of increasing seed protein content and/or increasing seed oil content of a soybean plant may comprise transforming the soybean plant with more than one polynucleotide encoding a soybean seed storage related promoter that functions in the soybean plant, provided that each polynucleotide encoding a soybean seed storage related promoter that function in the soybean plant is operably linked to a polynucleotide encoding a soybean seed storage polypeptide.

The more than one polynucleotide encoding a soybean seed storage related promoter may be selected from the group consisting of: (i) any wild type β-Conglycinin CoGy1 promoter sequence, or a sequence at least 95% identical thereto, or a full length complement thereof, or a functional fragment thereof, wherein the soybean seed storage polypeptide comprises the wild type “Forrest” β-Conglycinin CoGy1 sequence or the wild type “Williams 82” (3-Conglycinin CoGy1 sequence (SEQ ID NO: 57), or a sequence at least 95% identical thereto, or a full-length complement thereof, or a functional fragment thereof, and further comprises one or more mutations of the wild type “Forrest” β-Conglycinin CoGy1 sequence or the wild type “Williams 82” β-Conglycinin CoGy1 sequence (SEQ ID NO: 57) selected from the group consisting of: A4T, W28*, R231C, R247*, and R510Q; (ii) any wild type β-Conglycinin CoGy2 promoter sequence, or a sequence at least 95% identical thereto, or a full length complement thereof, or a functional fragment thereof, wherein the soybean seed storage polypeptide comprises the wild type “Forrest” β-Conglycinin CoGy2 sequence or the wild type “Williams 82” β-Conglycinin CoGy2 sequence (SEQ ID NO: 61), or a sequence at least 95% identical thereto, or a full-length complement thereof, or a functional fragment thereof, and further comprises one or more mutations of the wild type “Forrest” β-Conglycinin CoGy2 sequence or the wild type “Williams 82” β-Conglycinin CoGy2 sequence (SEQ ID NO: 61) selected from the group consisting of: S255N and R287K; (iii) any wild type β-Conglycinin CoGy3 promoter sequence, or a sequence at least 95% identical thereto, or a full length complement thereof, or a functional fragment thereof, wherein the soybean seed storage polypeptide comprises the wild type “Forrest” 3-Conglycinin CoGy3 sequence or the wild type “Williams 82” β-Conglycinin CoGy3 sequence (SEQ ID NO: 65), or a sequence at least 95% identical thereto, or a full-length complement thereof, or a functional fragment thereof, and further comprises one or more mutations of the wild type “Forrest” β-Conglycinin CoGy3 sequence or the wild type “Forrest” β-Conglycinin CoGy3 sequence (SEQ ID NO: 65); and (iv) any wild type Conglycinin CoGy4 promoter sequence, or a sequence at least 95% identical thereto, or a full length complement thereof, or a functional fragment thereof, wherein the soybean seed storage polypeptide comprises the wild type “Forrest” Conglycinin CoGy4 sequence or the wild type “Williams 82” β-Conglycinin CoGy4 sequence (SEQ ID NO: 69), or a sequence at least 95% identical thereto, or a full-length complement thereof, or a functional fragment thereof, and further comprises one or more mutations of the wild type “Forrest” Conglycinin CoGy4 sequence or the wild type “Williams 82” β-Conglycinin CoGy4 sequence (SEQ ID NO: 69) selected from the group consisting of: C39Y, D249D, D296N, and K461K.

The transformed soybean plant may have increased seed protein content as compared to a control soybean plant lacking the polynucleotide encoding a soybean seed storage polynucleotide as described above.

The transformed soybean plant may have increased seed oil content as compared to a control soybean plant lacking the polynucleotide encoding a soybean seed storage polynucleotide as described above.

The transformed soybean plant may have both increased seed protein content as compared to a control soybean plant lacking the polynucleotide encoding a soybean seed storage polynucleotide as described above and increased seed oil content as compared to a control soybean plant lacking the polynucleotide encoding a soybean seed storage polynucleotide as described above.

The increased seed protein content may comprise at least about 1%, at least about 2%, at least about 3%, at least about 4%, at least about 5%, at least about 6%, at least about 7%, at least about 8%, at least about 9%, at least about 10%, at least about 20%, at least about 30%, at least about 40%, at least about 50%, at least about 60%, at least about 70%, at least about 80%, at least about 90%, or at least about 100% increase in seed protein content as compared to the control soybean plant lacking the polynucleotide encoding a soybean seed storage polynucleotide as described above.

The increased seed oil content may comprise at least about 1%, at least about 2%, at least about 3%, at least about 4%, at least about 5%, at least about 6%, at least about 7%, at least about 8%, at least about 9%, at least about 10%, at least about 20%, at least about 30%, at least about 40%, at least about 50%, at least about 60%, at least about 70%, at least about 80%, at least about 90%, or at least about 100% increase in seed oil content as compared to the control soybean plant lacking the polynucleotide encoding a soybean seed storage polynucleotide as described above.

DNA Constructs

Another embodiment of the present invention is a DNA construct comprising a polynucleotide encoding a soybean seed storage related promoter that functions in the soybean plant operably linked to a polynucleotide encoding a soybean seed storage polypeptide.

The polynucleotide encoding a soybean seed storage related promoter may comprise any wild type soybean seed storage promoter sequence, or a sequence at least 95% identical thereto, or a full length complement thereof, or a functional fragment thereof.

The soybean seed storage polypeptide may comprise any wild type soybean seed storage sequence, or a sequence at least 95% identical thereto, or a full-length complement thereof, or a functional fragment thereof.

The polynucleotide encoding a soybean seed storage related promoter may comprise any wild type β-Conglycinin CoGy1 promoter sequence, or a sequence at least 95% identical thereto, or a full length complement thereof, or a functional fragment thereof. The soybean seed storage polynucleotide may comprise the wild type “Williams 82” β-Conglycinin CoGy1 promoter sequence (SEQ ID NO: 56), or a sequence at least 95% identical thereto, or a full-length complement thereof, or a functional fragment thereof.

The soybean seed storage polypeptide may comprise the wild type “Forrest” 3-Conglycinin CoGy1 sequence, or a sequence at least 95% identical thereto, or a full-length complement thereof, or a functional fragment thereof, and may further comprise one or more mutations of the wild type “Forrest” β-Conglycinin CoGy1 sequence selected from the group consisting of: A4T, W28*, R231C, R247*, and R510Q. The soybean seed storage polypeptide may comprise the wild type “Williams 82” β-Conglycinin CoGy1 sequence (SEQ ID NO: 57), or a sequence at least 95% identical thereto, or a full-length complement thereof, or a functional fragment thereof, and may further comprise one or more mutations of the wild type “Williams 82” 3-Conglycinin CoGy1 sequence selected from the group consisting of: A4T, W28*, R231C, R247*, and R510Q.

The polynucleotide encoding a soybean seed storage related polypeptide may comprise any wild type β-Conglycinin CoGy1 genomic or coding sequence, or a sequence at least 95% identical thereto, or a full length complement thereof, or a functional fragment thereof. The soybean seed storage polynucleotide may comprise the wild type “Williams 82” β-Conglycinin CoGy1 genomic (SEQ ID NO: 58) or coding (SEQ ID NO: 59) sequence, or a sequence at least 95% identical thereto, or a full-length complement thereof, or a functional fragment thereof, and may further comprise one or more mutations of the wild type “Williams 82” 3-Conglycinin CoGy1 genomic sequence (SEQ ID NO: 58) selected from the group consisting of: G10A, G84A, C691T, C739T, and G1529A.

The polynucleotide encoding a soybean seed storage related promoter may comprise any wild type β-Conglycinin CoGy2 promoter sequence, or a sequence at least 95% identical thereto, or a full length complement thereof, or a functional fragment thereof. The soybean seed storage polynucleotide may comprise the wild type “Williams 82” β-Conglycinin CoGy2 promoter sequence (SEQ ID NO: 60), or a sequence at least 95% identical thereto, or a full-length complement thereof, or a functional fragment thereof.

The soybean seed storage polypeptide may comprise the wild type “Forrest” 3-Conglycinin CoGy2 sequence, or a sequence at least 95% identical thereto, or a full-length complement thereof, or a functional fragment thereof, and may further comprise one or more mutations of the wild type “Forrest” β-Conglycinin CoGy2 sequence selected from the group consisting of: S255N and R287K. The soybean seed storage polypeptide may comprise the wild type “Williams 82” β-Conglycinin CoGy2 sequence (SEQ ID NO: 61), or a sequence at least 95% identical thereto, or a full-length complement thereof, or a functional fragment thereof, and may further comprise one or more mutations of the wild type “Williams 82” β-Conglycinin CoGy2 sequence selected from the group consisting of: S255N and R287K.

The polynucleotide encoding a soybean seed storage related polypeptide may comprise any wild type β-Conglycinin CoGy2 genomic or coding sequence, or a sequence at least 95% identical thereto, or a full length complement thereof, or a functional fragment thereof. The soybean seed storage polynucleotide may comprise the wild type “Williams 82” β-Conglycinin CoGy2 genomic (SEQ ID NO: 62) or coding (SEQ ID NO: 63) sequence, or a sequence at least 95% identical thereto, or a full-length complement thereof, or a functional fragment thereof, and may further comprise one or more mutations of the wild type “Williams 82” 3-Conglycinin CoGy2 genomic sequence (SEQ ID NO: 62) selected from the group consisting of: G764A and G860A.

The polynucleotide encoding a soybean seed storage related promoter may comprise any wild type β-Conglycinin CoGy3 promoter sequence, or a sequence at least 95% identical thereto, or a full length complement thereof, or a functional fragment thereof. The soybean seed storage polynucleotide may comprise the wild type “Williams 82” β-Conglycinin CoGy3 promoter sequence (SEQ ID NO: 64), or a sequence at least 95% identical thereto, or a full-length complement thereof, or a functional fragment thereof.

The soybean seed storage polypeptide may comprise the wild type “Forrest” 3-Conglycinin CoGy3 sequence, or a sequence at least 95% identical thereto, or a full-length complement thereof, or a functional fragment thereof, and may further comprise one or more mutations of the wild type “Forrest” β-Conglycinin CoGy3 sequence. The soybean seed storage polypeptide may comprise the wild type “Williams 82” β-Conglycinin CoGy3 sequence (SEQ ID NO: 65), or a sequence at least 95% identical thereto, or a full-length complement thereof, or a functional fragment thereof, and may further comprise one or more mutations of the wild type “Williams 82” β-Conglycinin CoGy3 sequence.

The polynucleotide encoding a soybean seed storage related polypeptide may comprise any wild type β-Conglycinin CoGy3 genomic or coding sequence, or a sequence at least 95% identical thereto, or a full length complement thereof, or a functional fragment thereof. The soybean seed storage polynucleotide may comprise the wild type “Williams 82” β-Conglycinin CoGy3 genomic (SEQ ID NO: 66) or coding (SEQ ID NO: 67) sequence, or a sequence at least 95% identical thereto, or a full-length complement thereof, or a functional fragment thereof, and may further comprise one or more mutations of the wild type “Williams 82” β-Conglycinin CoGy3 genomic sequence (SEQ ID NO: 66).

The polynucleotide encoding a soybean seed storage related promoter may comprise any wild type β-Conglycinin CoGy4 promoter sequence, or a sequence at least 95% identical thereto, or a full length complement thereof, or a functional fragment thereof. The soybean seed storage polynucleotide may comprise the wild type “Williams 82” β-Conglycinin CoGy4 promoter sequence (SEQ ID NO: 68), or a sequence at least 95% identical thereto, or a full-length complement thereof, or a functional fragment thereof.

The soybean seed storage polypeptide may comprise the wild type “Forrest” 3-Conglycinin CoGy4 sequence, or a sequence at least 95% identical thereto, or a full-length complement thereof, or a functional fragment thereof, and may further comprise one or more mutations of the wild type “Forrest” Conglycinin α′-Subunit β-Conglycinin CoGy4 sequence selected from the group consisting of: C39Y, D249D, D296N, and K461K. The soybean seed storage polypeptide may comprise the wild type “Williams 82” β-Conglycinin CoGy4 sequence (SEQ ID NO: 69), or a sequence at least 95% identical thereto, or a full-length complement thereof, or a functional fragment thereof, and may further comprise one or more mutations of the wild type “Williams 82” β-Conglycinin CoGy4 sequence selected from the group consisting of: C39Y, D249D, D296N, and K461K.

The polynucleotide encoding a soybean seed storage related polypeptide may comprise any wild type β-Conglycinin CoGy4 genomic or coding sequence, or a sequence at least 95% identical thereto, or a full length complement thereof, or a functional fragment thereof. The soybean seed storage polynucleotide may comprise the wild type “Williams 82” β-Conglycinin CoGy4 genomic (SEQ ID NO: 70) or coding (SEQ ID NO: 71) sequence, or a sequence at least 95% identical thereto, or a full-length complement thereof, or a functional fragment thereof, and may further comprise one or more mutations of the wild type “Williams 82” 3-Conglycinin CoGy4 genomic sequence (SEQ ID NO: 70) selected from the group consisting of: G116A, C747T, G886A, and G1383A.

The DNA construct may comprise more than one polynucleotide encoding a soybean seed storage related promoter that functions in the soybean plant, provided that each polynucleotide encoding a soybean seed storage related promoter that function in the soybean plant is operably linked to a polynucleotide encoding a soybean seed storage polypeptide.

The more than one polynucleotide encoding a soybean seed storage related promoter may be selected from the group consisting of: (i) any wild type β-Conglycinin CoGy1 promoter sequence, or a sequence at least 95% identical thereto, or a full length complement thereof, or a functional fragment thereof, wherein the soybean seed storage polypeptide comprises the wild type “Forrest” β-Conglycinin CoGy1 sequence or the wild type “Williams 82” β-Conglycinin CoGy1 sequence (SEQ ID NO: 57), or a sequence at least 95% identical thereto, or a full-length complement thereof, or a functional fragment thereof, and further comprises one or more mutations of the wild type “Forrest” β-Conglycinin CoGy1 sequence or the wild type “Williams 82” β-Conglycinin CoGy1 sequence (SEQ ID NO: 57) selected from the group consisting of: A4T, W28*, R231C, R247*, and R510Q; (ii) any wild type β-Conglycinin CoGy2 promoter sequence, or a sequence at least 95% identical thereto, or a full length complement thereof, or a functional fragment thereof, wherein the soybean seed storage polypeptide comprises the wild type “Forrest” β-Conglycinin CoGy2 sequence or the wild type “Williams 82” β-Conglycinin CoGy2 sequence (SEQ ID NO: 61), or a sequence at least 95% identical thereto, or a full-length complement thereof, or a functional fragment thereof, and further comprises one or more mutations of the wild type “Forrest” β-Conglycinin CoGy2 sequence or the wild type “Williams 82” β-Conglycinin CoGy2 sequence (SEQ ID NO: 61) selected from the group consisting of: S255N and R287K; (iii) any wild type β-Conglycinin CoGy3 promoter sequence, or a sequence at least 95% identical thereto, or a full length complement thereof, or a functional fragment thereof, wherein the soybean seed storage polypeptide comprises the wild type “Forrest” 3-Conglycinin CoGy3 sequence or the wild type “Williams 82” β-Conglycinin CoGy3 sequence (SEQ ID NO: 65), or a sequence at least 95% identical thereto, or a full-length complement thereof, or a functional fragment thereof, and further comprises one or more mutations of the wild type “Forrest” β-Conglycinin CoGy3 sequence or the wild type “Williams 82” β-Conglycinin CoGy3 sequence (SEQ ID NO: 65); and (iv) any wild type Conglycinin CoGy4 promoter sequence, or a sequence at least 95% identical thereto, or a full length complement thereof, or a functional fragment thereof, wherein the soybean seed storage polypeptide comprises the wild type “Forrest” Conglycinin CoGy4 sequence or the wild type “Williams 82” β-Conglycinin CoGy4 sequence (SEQ ID NO: 69), or a sequence at least 95% identical thereto, or a full-length complement thereof, or a functional fragment thereof, and further comprises one or more mutations of the wild type “Forrest” Conglycinin CoGy4 sequence or the wild type “Williams 82” β-Conglycinin CoGy4 sequence (SEQ ID NO: 69) selected from the group consisting of: C39Y, D249D, D296N, and K461K.

Sequences and Mutations

The amino acid sequences and nucleic acid sequences described herein may contain various mutations. Mutations may include insertions, substitutions, and deletions. Insertions are written as follows: (+)(amino acid/nucleic acid sequence position number)(inserted amino acid/nucleic acid base). For example, +287A would mean an insertion of an alanine residue after position 287 in the corresponding amino acid sequence. Substitutions are written as follows: (amino acid/nucleic acid base to be replaced)(amino acid/nucleic acid sequence position number)(substituted amino acid/nucleic acid base). For example, C1082A would mean a substitution of an adenine base instead of a cytosine base at position 1082 in the corresponding nucleic acid sequence. Deletions are written as follows: (amino acid/nucleic acid base to be deleted)(amino acid/nucleic acid sequence position number)(−). For example, C970− would mean a deletion of the cytosine base normally located at position 970 in the corresponding nucleic acid sequence. “*” can also be used to indicate a deletion or premature stop.

The amino acid sequences and nucleic acid sequences described herein may contain mutations at various sequence positions. Sequence positions may be written a variety a ways for convenience. More specifically, sequence positions may be written from either the beginning of the sequence as a positive position number, or from the end of the sequence as a negative number. Sequence positions may be converted easily between a positive notation and a negative notation by comparing to the sequence length and either adding or subtracting the sequence length. For example, a promoter containing 10 nucleic acid bases with a mutation from cytosine to adenine at the second position from the start of the sequence may be written as C2A. Alternatively, this mutation may be written as C(−9)A, −9C/A, or in a similar fashion denoting the negative position number.

### Definitions and Alternate Embodiments

The following definitions and methods are provided to better define the present invention and to guide those of ordinary skill in the art in the practice of the present invention. Unless otherwise noted, terms are to be understood according to conventional usage by those of ordinary skill in the relevant art.

The term “agronomically elite” refers to a genotype that has a culmination of many distinguishable traits such as emergence, vigor, vegetative vigor, disease resistance, seed set, standability, and threshability, which allows a producer to harvest a product of commercial significance.

An “allele” refers to one of two or more alternative forms of a genomic sequence at a given locus on a chromosome.

The term “chimeric” is understood to refer to the product of the fusion of portions of two or more different polynucleotide molecules. “Chimeric promoter” is understood to refer to a promoter produced through the manipulation of known promoters or other polynucleotide molecules. Such chimeric promoters can combine enhancer domains that can confer or modulate gene expression from one or more promoters or regulatory elements, for example, by fusing a heterologous enhancer domain from a first promoter to a second promoter with its own partial or complete regulatory elements. Thus, the design, construction, and use of chimeric promoters according to the methods disclosed herein for modulating the expression of operably linked polynucleotide sequences are encompassed by the present invention.

Novel chimeric promoters can be designed or engineered by a number of methods. For example, a chimeric promoter may be produced by fusing an enhancer domain from a first promoter to a second promoter. The resultant chimeric promoter may have novel expression properties relative to the first or second promoters. Novel chimeric promoters can be constructed such that the enhancer domain from a first promoter is fused at the 5′ end, at the 3′ end, or at any position internal to the second promoter.

A “construct” is generally understood as any recombinant nucleic acid molecule such as a plasmid, cosmid, virus, autonomously replicating nucleic acid molecule, phage, or linear or circular single-stranded or double-stranded DNA or RNA nucleic acid molecule, derived from any source, capable of genomic integration or autonomous replication, comprising a nucleic acid molecule where one or more nucleic acid molecule has been operably linked.

A construct of the present invention can contain a promoter operably linked to a transcribable nucleic acid molecule operably linked to a 3′ transcription termination nucleic acid molecule. In addition, constructs can include but are not limited to additional regulatory nucleic acid molecules from, e.g., the 3′-untranslated region (3′ UTR). Constructs can include but are not limited to the 5′ untranslated regions (5′ UTR) of an mRNA nucleic acid molecule, which can play an important role in translation initiation and can also be a genetic component in an expression construct. These additional upstream and downstream regulatory nucleic acid molecules may be derived from a source that is native or heterologous with respect to the other elements present on the promoter construct.

“Expression vector”, “vector”, “expression construct”, “vector construct”, “plasmid”, or “recombinant DNA construct” is generally understood to refer to a nucleic acid that has been generated via human intervention, including by recombinant means or direct chemical synthesis, with a series of specified nucleic acid elements that permit transcription or translation of a particular nucleic acid in, for example, a host cell. The expression vector can be part of a plasmid, virus, or nucleic acid fragment. Typically, the expression vector can include a nucleic acid to be transcribed operably linked to a promoter.

The term “genotype” means the specific allelic makeup of a plant.

The terms “heterologous DNA sequence”, “exogenous DNA segment” or “heterologous nucleic acid,” as used herein, each refer to a sequence that originates from a source foreign to the particular host cell or, if from the same source, is modified from its original form. Thus, a heterologous gene in a host cell includes a gene that is endogenous to the particular host cell but has been modified through, for example, the use of DNA shuffling. The terms also include non-naturally occurring multiple copies of a naturally occurring DNA sequence. Thus, the terms refer to a DNA segment that is foreign or heterologous to the cell, or homologous to the cell but in a position within the host cell nucleic acid in which the element is not ordinarily found. Exogenous DNA segments are expressed to yield exogenous polypeptides. A “homologous” DNA sequence is a DNA sequence that is naturally associated with a host cell into which it is introduced.

“Highly stringent hybridization conditions” are defined as hybridization at 65° C. in a 6×SSC buffer (i.e., 0.9 M sodium chloride and 0.09 M sodium citrate). Given these conditions, a determination can be made as to whether a given set of sequences will hybridize by calculating the melting temperature (Tₘ) of a DNA duplex between the two sequences. If a particular duplex has a melting temperature lower than 65° C. in the salt conditions of a 6×SSC, then the two sequences will not hybridize. On the other hand, if the melting temperature is above 65° C. in the same salt conditions, then the sequences will hybridize. In general, the melting temperature for any hybridized DNA:DNA sequence can be determined using the following formula: Tₘ=81.5° C.+16.6(log₁₀[Na^{+]})+0.41(fraction G/C content)−0.63(% formamide)−(600/1). Furthermore, the Tₘ of a DNA:DNA hybrid is decreased by 1-1.5° C. for every 1% decrease in nucleotide identity.

The term “introgressed,” when used in reference to a genetic locus, refers to a genetic locus that has been introduced into a new genetic background. Introgression of a genetic locus can thus be achieved through plant breeding methods and/or by molecular genetic methods. Such molecular genetic methods include, but are not limited to, various plant transformation techniques and/or methods that provide for homologous recombination, non-homologous recombination, site-specific recombination, and/or genomic modifications that provide for locus substitution or locus conversion.

The term “linked,” when used in the context of nucleic acid markers and/or genomic regions, means that the markers and/or genomic regions are located on the same linkage group or chromosome.

A “marker” means a detectable characteristic that can be used to discriminate between organisms. Examples of such characteristics include, but are not limited to, genetic markers, biochemical markers, metabolites, morphological characteristics, and agronomic characteristics.

A “marker gene” refers to any transcribable nucleic acid molecule whose expression can be screened for or scored in some way.

Certain genetic markers useful in the present invention include “dominant” or “codominant” markers. “Codominant” markers reveal the presence of two or more alleles (two per diploid individual). “Dominant” markers reveal the presence of only a single allele. The presence of the dominant marker phenotype (e.g., a band of DNA) is an indication that one allele is present in either the homozygous or heterozygous condition. The absence of the dominant marker phenotype (e.g., absence of a DNA band) is merely evidence that “some other” undefined allele is present. In the case of populations where individuals are predominantly homozygous and loci are predominantly dimorphic, dominant and codominant markers can be equally valuable. As populations become more heterozygous and multiallelic, codominant markers often become more informative of the genotype than dominant markers.

“Operably-linked” or “functionally linked” refers preferably to the association of nucleic acid sequences on a single nucleic acid fragment so that the function of one is affected by the other. For example, a regulatory DNA sequence is said to be “operably linked to” or “associated with” a DNA sequence that codes for an RNA or a polypeptide if the two sequences are situated such that the regulatory DNA sequence affects expression of the coding DNA sequence (i.e., that the coding sequence or functional RNA is under the transcriptional control of the promoter). Coding sequences can be operably-linked to regulatory sequences in sense or antisense orientation. The two nucleic acid molecules may be part of a single contiguous nucleic acid molecule and may be adjacent. For example, a promoter is operably linked to a gene of interest if the promoter regulates or mediates transcription of the gene of interest in a cell.

The term “phenotype” means the detectable characteristics of a cell or organism that can be influenced by gene expression.

The term “plant” can include plant cells, plant protoplasts, plant cells of tissue culture from which a plant can be regenerated, plant calli, plant clumps and plant cells that are intact in plants or parts of plants such as pollen, flowers, seeds, leaves, stems, and the like. Each of these terms can apply to a soybean “plant”. Plant parts (e.g., soybean parts) include, but are not limited to, pollen, an ovule and a cell.

The term “population” means a genetically heterogeneous collection of plants that share a common parental derivation.

A “promoter” is generally understood as a nucleic acid control sequence that directs transcription of a nucleic acid. An inducible promoter is generally understood as a promoter that mediates transcription of an operably linked gene in response to a particular stimulus. A promoter can include necessary nucleic acid sequences near the transcription start site, such as, in the case of a polymerase II type promoter, a TATA element. A promoter can optionally include distal enhancer or repressor elements, which can be located as much as several thousand base pairs from the start site of transcription.

A “quantitative trait locus (QTL)” is a chromosomal location that encodes for alleles that affect the expressivity of a phenotype.

A “transcribable nucleic acid molecule” as used herein refers to any nucleic acid molecule capable of being transcribed into a RNA molecule. Methods are known for introducing constructs into a cell in such a manner that the transcribable nucleic acid molecule is transcribed into a functional mRNA molecule that is translated and therefore expressed as a protein product. Constructs may also be constructed to be capable of expressing antisense RNA molecules, in order to inhibit translation of a specific RNA molecule of interest. For the practice of the present invention, conventional compositions and methods for preparing and using constructs and host cells may be used.

The “transcription start site” or “initiation site” is the position surrounding a nucleotide that is part of the transcribed sequence, which is also defined as position+1. With respect to this site all other sequences of the gene and its controlling regions can be numbered. Downstream sequences (i.e., further protein encoding sequences in the 3′ direction) can be denominated positive, while upstream sequences (mostly of the controlling regions in the 5′ direction) can be denominated as negative.

The term “transformation” refers to the transfer of a nucleic acid fragment into the genome of a host cell, resulting in genetically stable inheritance. Host cells containing the transformed nucleic acid fragments are referred to as “transgenic” cells, and organisms comprising transgenic cells are referred to as “transgenic organisms”.

“Transformed,” “transgenic,” and “recombinant” refer to a host cell or organism such as a plant into which a heterologous nucleic acid molecule has been introduced. The nucleic acid molecule can be stably integrated into the genome as generally known in the art. Known methods of PCR include, but are not limited to, methods using paired primers, nested primers, single specific primers, degenerate primers, gene-specific primers, vector-specific primers, partially mismatched primers, and the like. The term “untransformed” refers to normal cells that have not been through the transformation process.

The terms “variety” and “cultivar” mean a group of similar plants that by their genetic pedigrees and performance can be identified from other varieties within the same species.

“Wild-type” refers to a virus or organism found in nature without any known mutation.

In some embodiments, numbers expressing quantities of ingredients, properties such as molecular weight, reaction conditions, and so forth, used to describe and claim certain embodiments of the present invention are to be understood as being modified in some instances by the term “about.” In some embodiments, the term “about” is used to indicate that a value includes the standard deviation of the mean for the device or method being employed to determine the value. In some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the present invention are approximations, the numerical values set forth in the specific examples are reported as precisely as practicable. The numerical values presented in some embodiments of the present invention may contain certain errors necessarily resulting from the standard deviation found in their respective testing measurements. The recitation of ranges of values herein is merely intended to serve as a shorthand method of referring individually to each separate value falling within the range. Unless otherwise indicated herein, each individual value is incorporated into the specification as if it were individually recited herein.

Nucleotide and/or amino acid sequence identity percent (%) is understood as the percentage of nucleotide or amino acid residues that are identical with nucleotide or amino acid residues in a candidate sequence in comparison to a reference sequence when the two sequences are aligned. To determine percent identity, sequences are aligned and if necessary, gaps are introduced to achieve the maximum percent sequence identity. Sequence alignment procedures to determine percent identity are well known to those of skill in the art. Often publicly available computer software such as BLAST, BLAST2, ALIGN2 or Megalign (DNASTAR) software is used to align sequences. Those skilled in the art can determine appropriate parameters for measuring alignment, including any algorithms needed to achieve maximal alignment over the full-length of the sequences being compared. When sequences are aligned, the percent sequence identity of a given sequence A to, with, or against a given sequence B (which can alternatively be phrased as a given sequence A that has or comprises a certain percent sequence identity to, with, or against a given sequence B) can be calculated as: percent sequence identity=X/Y100, where X is the number of residues scored as identical matches by the sequence alignment program's or algorithm's alignment of A and B and Y is the total number of residues in B. If the length of sequence A is not equal to the length of sequence B, the percent sequence identity of A to B will not equal the percent sequence identity of B to A.

In some embodiments, the terms “a” and “an” and “the” and similar references used in the context of describing a particular embodiment (especially in the context of certain of the following claims) can be construed to cover both the singular and the plural, unless specifically noted otherwise. When used in conjunction with the word “comprising” or other open language in the claims, the words “a” and “an” denote “one or more,” unless specifically noted.

In some embodiments, the term “or” as used herein, including the claims, is used to mean “and/or” unless explicitly indicated to refer to alternatives only or the alternatives are mutually exclusive.

The terms “comprise,” “have” and “include” are open-ended linking verbs. Any forms or tenses of one or more of these verbs, such as “comprises,” “comprising,” “has,” “having,” “includes” and “including,” are also open-ended. For example, any method that “comprises,” “has” or “includes” one or more steps is not limited to possessing only those one or more steps and can also cover other unlisted steps. Similarly, any composition or device that “comprises,” “has” or “includes” one or more features is not limited to possessing only those one or more features and can cover other unlisted features.

All methods described herein can be performed in any suitable order unless otherwise indicated herein or otherwise clearly contradicted by context. The use of any and all examples, or exemplary language (e.g. “such as”) provided with respect to certain embodiments herein is intended merely to better illuminate the present invention and does not pose a limitation on the scope of the present invention otherwise claimed. No language in the specification should be construed as indicating any non-claimed element essential to the practice of the present invention.

Groupings of alternative elements or embodiments of the present invention disclosed herein are not to be construed as limitations. Each group member can be referred to and claimed individually or in any combination with other members of the group or other elements found herein. One or more members of a group can be included in, or deleted from, a group for reasons of convenience or patentability. When any such inclusion or deletion occurs, the specification is herein deemed to contain the group as modified thus fulfilling the written description of all Markush groups used in the appended claims.

All publications, patents, patent applications, and other references cited in this application are incorporated herein by reference in their entirety for all purposes to the same extent as if each individual publication, patent, patent application or other reference was specifically and individually indicated to be incorporated by reference in its entirety for all purposes. Citation of a reference herein shall not be construed as an admission that such is prior art to the present invention.

Having described the present invention in detail, it will be apparent that all of the compositions and methods disclosed and claimed herein can be made and executed without undue experimentation in light of the present invention. While the compositions and methods of this invention have been described in terms of preferred embodiments, it will be apparent to those of skill in the art that variations may be applied to the compositions and methods and in the steps or in the sequence of steps of the methods described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims. Furthermore, it should be appreciated that all examples in the present invention are provided as non-limiting examples.

### EXAMPLES

The following non-limiting examples are provided to further illustrate the present invention. It should be appreciated by those of skill in the art that the techniques disclosed in the examples that follow represent approaches the inventors have found function well in the practice of the present invention, and this can be considered to constitute examples of modes for its practice. However, those of skill in the art should, in light of the present invention, appreciate that many changes can be made in the specific embodiments that are disclosed and still obtain a like or similar result without departing from the spirit and scope of the present invention.

### Example 1. Development of an EMS Mutagenized Forrest Population

The soybean cv. “Forrest” seed was obtained from Southern Illinois University Carbondale Agricultural Research Center, and used to develop an EMS mutagenized population. The wild type “Forrest” seeds were mutagenized with 0.6% EMS, and planted to harvest 4032 M2 families, which was successively advanced to the M3 generation at Southern Illinois University Carbondale.

### Example 2. TILLING by Target Capture Sequencing

A two-dimensional pooling strategy was implemented to reduce the number of pools. Samples were vertically pooled into pools of 24 samples and horizontally into pools of 48 samples. Next, specific probes were designed to target regions of interest. After library preparation and probe design, the developed TILLING by Target Capture Sequencing workflow (based on capture-seq enrichment and target recovery technology) uses magnetic beads for targeting desired genes with higher efficiency and specificity before proceeding with next generation sequencing of the pooled DNA. The obtained result was saved as FASTQ file, data were then cleaned and filtered, and the resulting VCF (Variant Call Format) files were analyzed.

From next-generation sequencing, the data were transformed into VCF files through a bioinformatic process. Using the VCF files, the SNPs between Forrest and mutants within the mutant population were found. An R script was developed to select data based on the desired thresholds and conditions. This reduced the time to analyze the data as well as human errors involved in manual processing. After the identification of the desired mutants carrying the mutations, their seeds were phenotyped for the different seed composition traits.

### Example 3. Multiple Sequence Alignment

The β-Conglycinin sequences used in the current study were retrieved from Phytozome. Only β-Conglycinins containing two Cupin domains were selected. A consensus sequence was constructed using Cons tool from EMBOSS suite based on the four highly expressed β-Conglycinins. The resulting sequence was blasted in the Uniprot database to retrieve ortholog sequences from other species. Twenty-five sequences were collected from 20 species including model plants; Arabidopsis thaliana (eudicot), Oryza sativa (monocot), Amborella trichopoda (Basel Angiosperm), Selaginella moellendorfii (Lycophyte). The Multiple Sequence Alignment was done on 40 sequences including six β-Conglycinins EMS mutants and nine β-Conglycinins wyld-type sequences retrieved from Soybean genome. The alignment was conducted using MUSCLE algorithm included in the Jalview Software.

### Example 4. Phylogenetic Tree

Multiple sequence alignments of the retrieved Conglycinins from eudicots, monocots, basel angiosperm, and lycophyte were performed using the MEGA4 software package and the ClustalW algorithm, and calculated using the neighbor-joining method. The tree bootstrap values are indicated at the nodes (n=5000).

### Example 5. Syntenic Analysis

Syntenic Analysis was performed using Persephone software on the soybean genome. The syntenic chromosomes were selected based on a syntenic matrix showing the chromosomes with duplicated fragments. The chromosome maps were constructed and information about the homologous genes and the duplicated regions were collected.

### Example 6. Homology Modeling of GmCoGy Protein and Mutational Analysis

Homology modeling of putative CoGy1 and CoGy2 protein structures was conducted with Deepview and Swiss Model Workspace software using the protein sequence from “Williams 82” and an available crystal structure as a template; PDB accession 1UIK.1.A for CoGy1, 614C.1.A for CoGy2 and CoGy3, and 2ea7.1.A for CoGy4. All residues were modeled against the two templates with a sequence identity of 100%, 50.97%, 50.61%, 65.12% for CoGy1, CoGy2, CoGy3, and CoGy4 sequences, respectively. Mutation mapping and visualizations were performed using the UCSF Chimera package.

### Example 7. Seed Protein Extraction

For seed protein extraction, five seeds from each line were ground. 100 mg of seed powder was then homogenized with 5 ml of a solution containing 10% (w/v) trichloroacetic acid (TCA) in acetone with 0.07% (v/v) 2-mercaptoethanol to precipitate total protein for 1 h (or overnight) at −20° C. The extract was then centrifuged for 20 min at 20800 g and 4° C. Next, the pellet was washed three times with acetone containing 0.07% (v/v) 2-mercaptoethanol and then dried under vacuum for 30 min. The dry powder was resuspended in 1 ml of lysis Buffer (9M urea, 1% CHAPS, 1% [w/v] ampholytes [pH 3-10], 1% DTT), then shacked on ice for 30 min. The insoluble material was removed by centrifugation for 20 min at 20800 g and 4° C. The final supernatant was used for 2D-PAGE analysis.

### Example 8. Two-Dimensional Electrophoresis Conditions

Two-dimensional polyacrylamide gel electrophoresis was performed using the Bio-Rad IsoElectric focusing (IEF) System and protocol. 30 μg of protein extract was diluted in 200 μl of rehydration buffer and applied on a 3-10 pH gradient strip. The stripes were actively rehydrated at 50V for 12 h followed by a rapid increase to 250V for 15 min then a gradual increase to 4000V for 2 h. The value of 4000V was maintained for 9.5 hours. The strips were reduced with equilibration buffer I (Containing DTT) and alkylated with equilibration buffer II (containing iodoacetamide). The strips were sealed to 3-15% acrylamide gradient gels, then a regular electrophoresis at 12.5% polyacrylamide gel was performed at 200V for 35 min, and the gels were stained using Coomassie Blue for 4 min, before distaining for 30 min.

### Example 9. Forrest RNA-Seq Library Preparation and Analysis

Four plant soybean tissues were used for RNA-seq including seed, leaf, root, flower and pods. Total RNA of each sample was extracted from 100 mg of frozen grounded samples using RNeasy QIAGEN KIT (Cat. No./ID: 74004). Total RNA was treated with DNase I (Invitrogen, Carlsbad, Calif., USA). RNA-seq libraries preparation and sequencing were performed at Novogene INC. using Illumina NovaSeq 6000. The four libraries were multiplexed and sequenced in two different lanes generating 20 million raw pair end reads per sample (150 bp). Quality assessment of sequenced reads was performed using fastqc, version 0.11.9. After removing the low-quality reads and adapters with trimmomatic, version V0.39, the remaining high-quality reads were mapped to the soybean reference genome Wm82.a2.v1 using STAR, version v2.7.9. Uniquely mapped reads were counted using Python package HTseq v0.13.5. Read count normalization and differential gene expression analysis were conducted using the Deseq2 package v1.30.1 integrated in the OmicsBox platform from BioBam (Valencia, Spain).

### Example 10. Duplication of β-ConGlycinin Genes in Soybean Genome

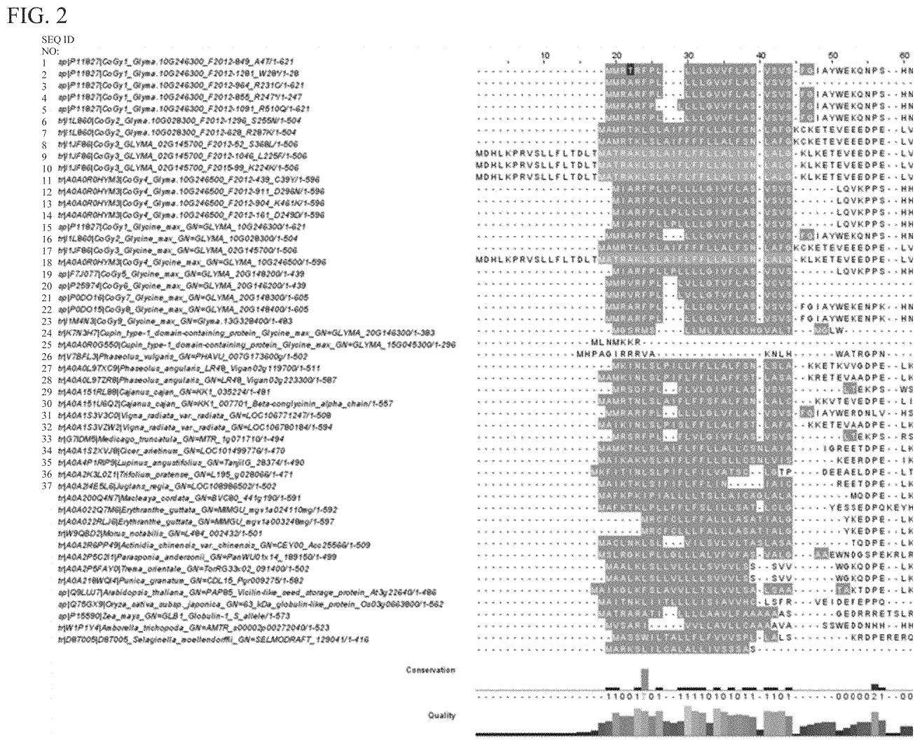

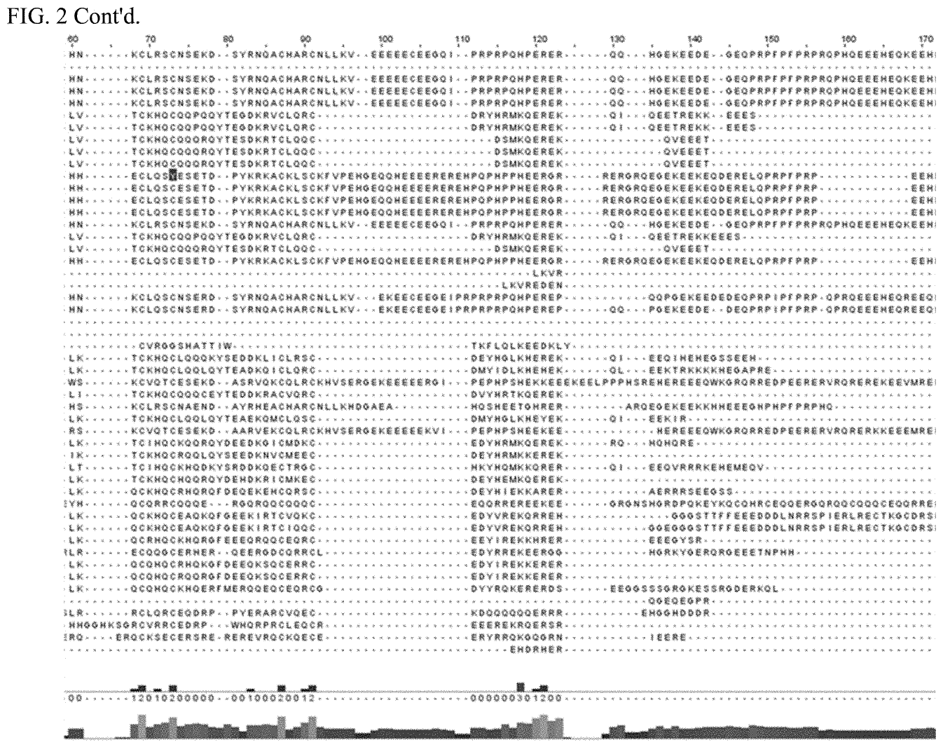

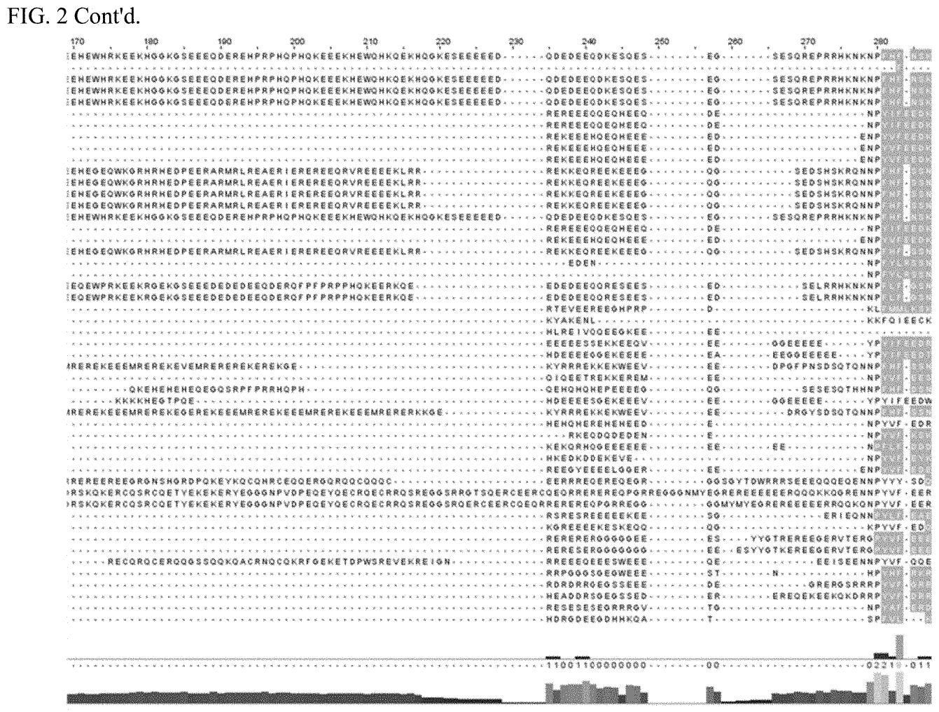

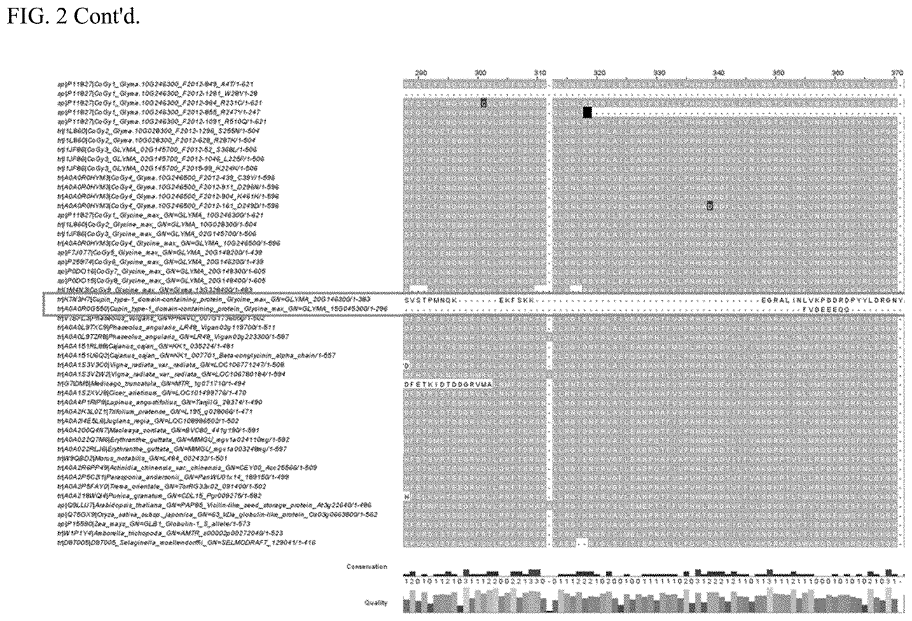

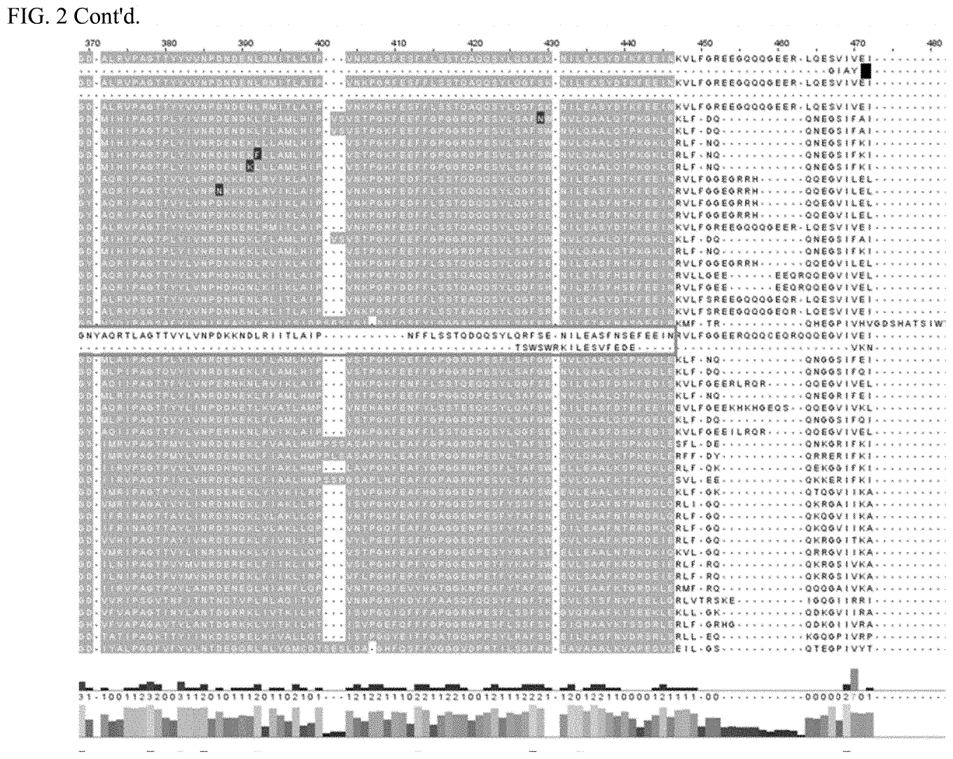

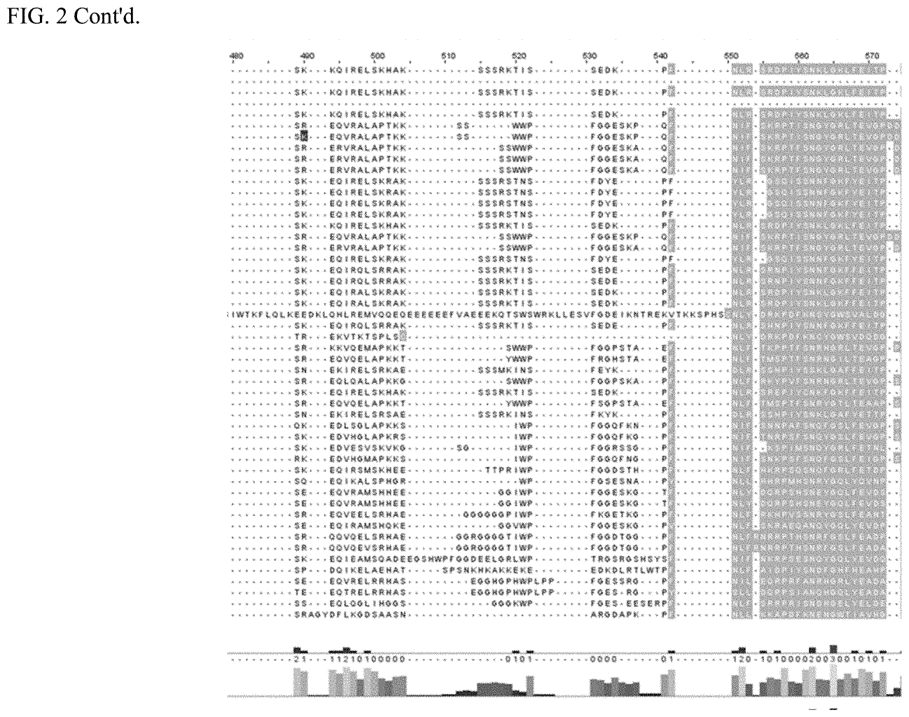

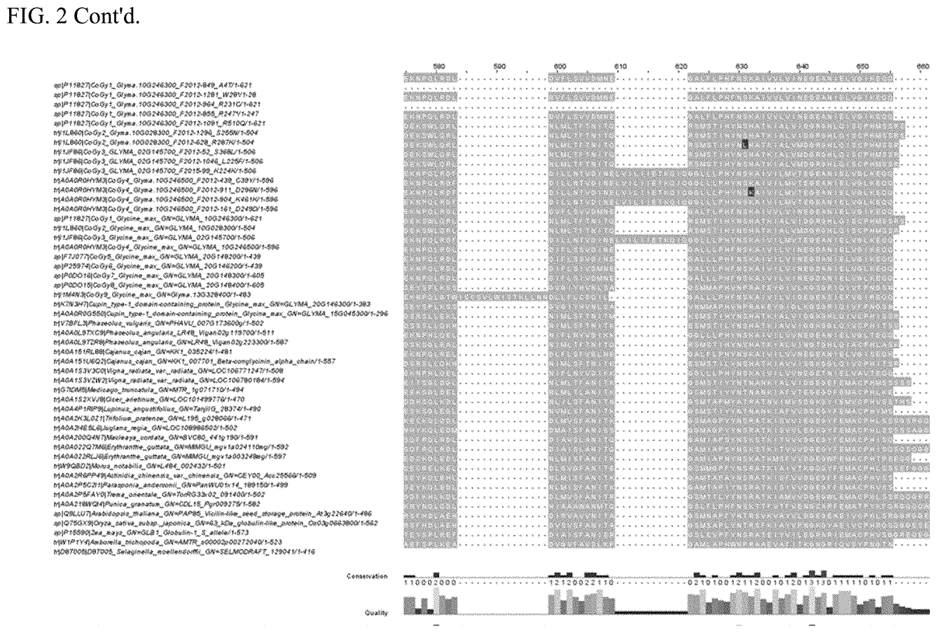

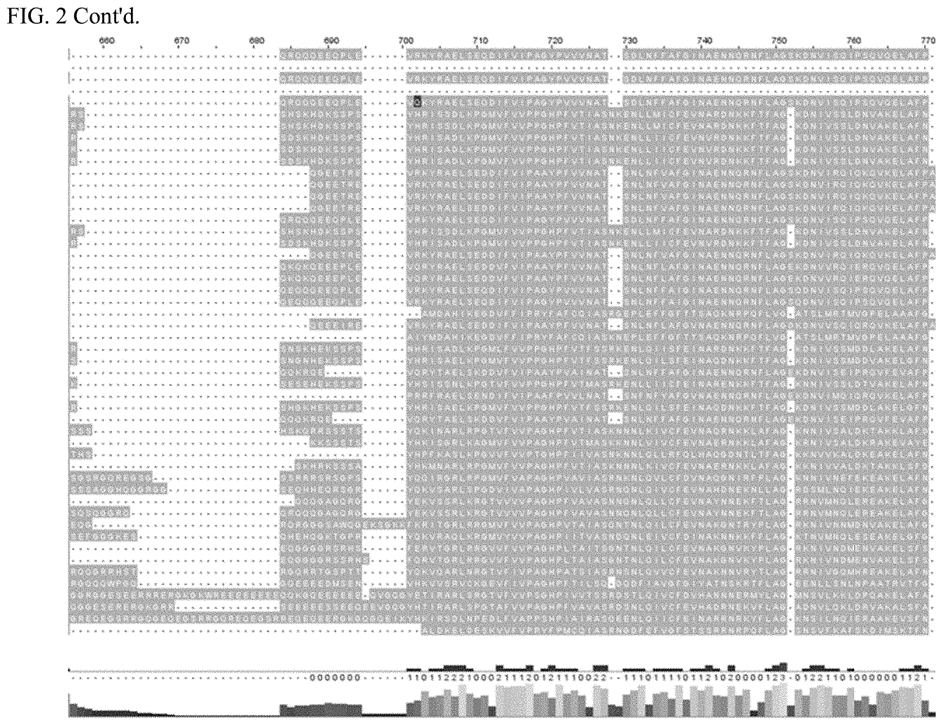

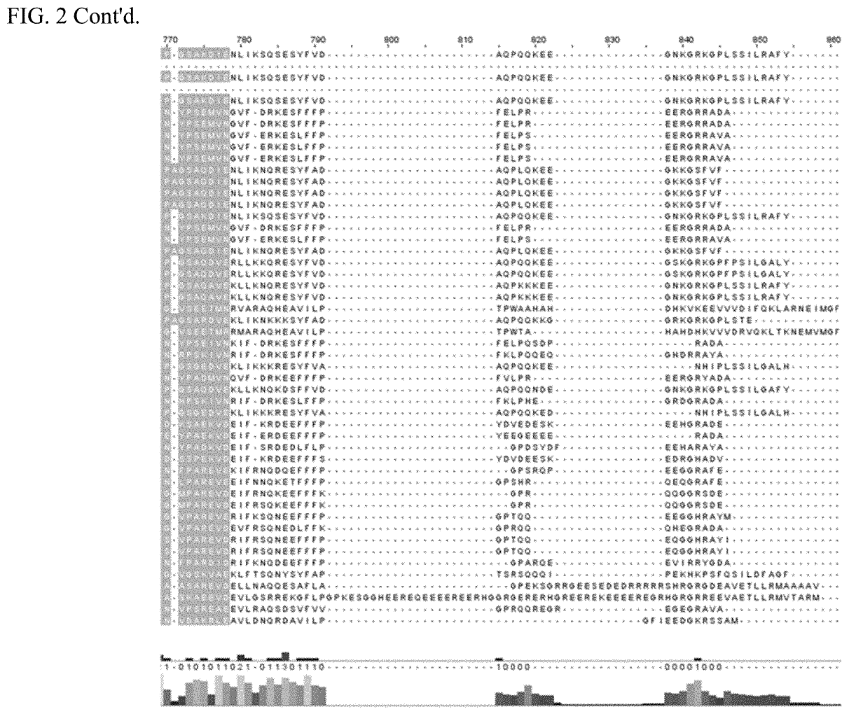

β-ConGlycinin constitute a family of proteins with a common modular architecture containing two conserved Cupin domains and distributed in specific positions throughout the β-ConGlycinin sequence and are involved in protein storage function (see FIG. 2). Extensive searches employing a variety of sequenced genomes using the typical distribution of the two Cupin domains of the soybean β-ConGlycinin failed to identify members of this protein family in primitive species including green algae, mosses, or seedless plants. However, β-ConGlycinins were found in all plant genomes analyzed, including lycophyte (Selaginella moellendorfii), basal angiosperms (Amborella trichopoda), monocots, and eudicots.

FIG. 3 shows a diagram depicting the phylogenetic tree of the β-Conglycinin subunits from 21 plant species including six model plants; A. trichopoda (angiosperm), S. moellendorfii (lycophyte), M. truncatula (leguminous), O. sativa (monocots), Z. mays (monocots), and A. thaliana (dicots), in addition to G. max (soybean) highlighted. The phylogenetic tree was generated using the MEGA4 software package and the ClustalW algorithm, and calculated using the neighbor-joining method. The tree bootstrap values are indicated at the nodes (n=5000). The analysis separately grouped monocot and dicot, and as expected, the ancestral β-ConGlycinins from the most primitive plant species Selaginella moellendorfii was out-grouped. Within the Eudicot clade, the eight expressed β-ConGlycinins were regrouped together and imbedded in two subclades containing β-ConGlycinins from other leguminous including pigeon pea, red bean, and mung bean. Because β-ConGlycinins appears to be commonly present in most seed plants analyzed, but absent in seedless plants, most likely, it's believed that β-ConGlycinins were part of seed speciation during the evolution to assure protein storage process that are needed to supply the embryo with the nutrition supply that are mobilized during seed germination. CoGy9 and Glyma.15g045300 were regrouped separately from the eudicot and monocot clades.

Investigation of the Williams 82 soybean genome indicated that the β-ConGlycinin gene family is composed of 9 members (see FIG. 4). Three are located on chromosome 10 named GmCoGy1, GmCoGy2, and GmCoGy4, one on chromosome 02 named GmCoGy3, four on chromosome 20 named GmCoGy5, GmCoGy6, GmCoGy7, and GmCoGy8, and one on chromosome 13 named GmCoGy9 (see FIG. 5), encoding proteins between 439 and 621 amino acid length (see FIG. 4).

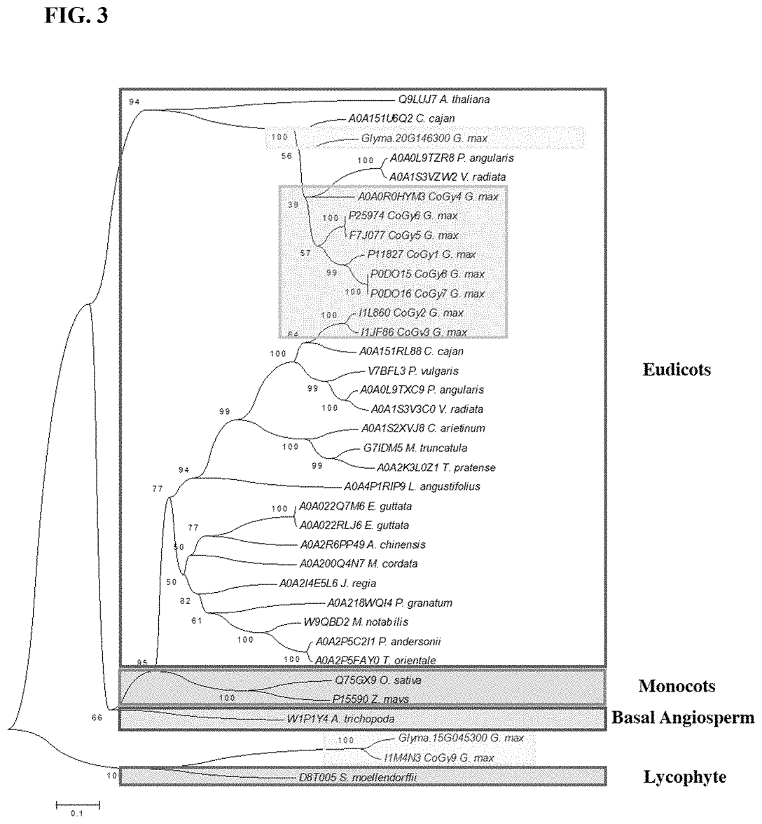

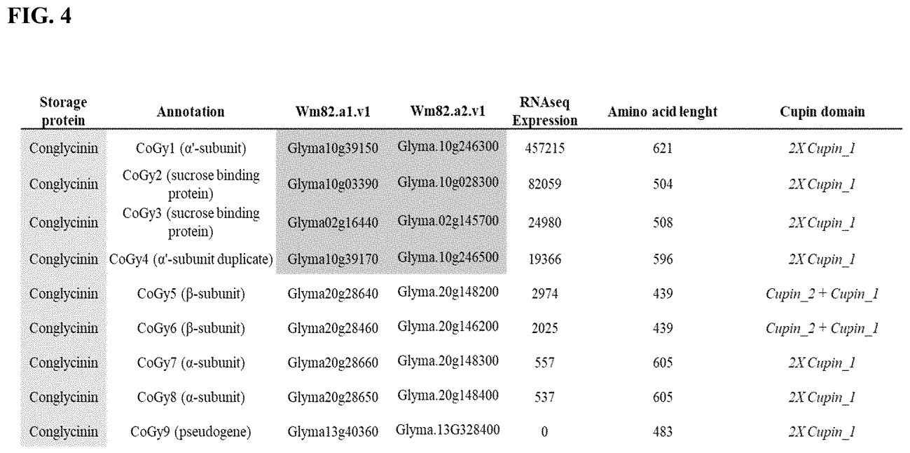

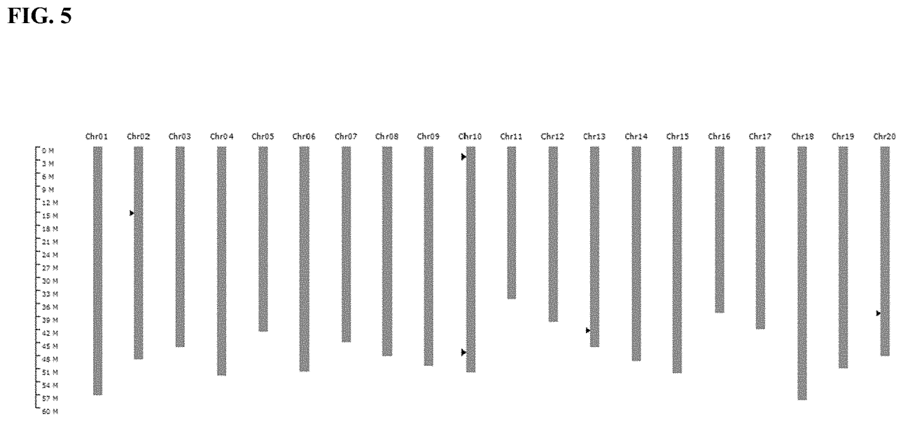

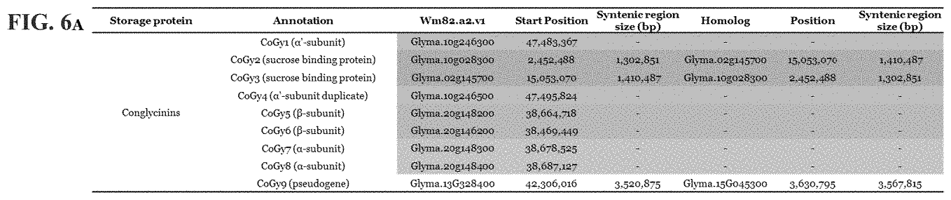

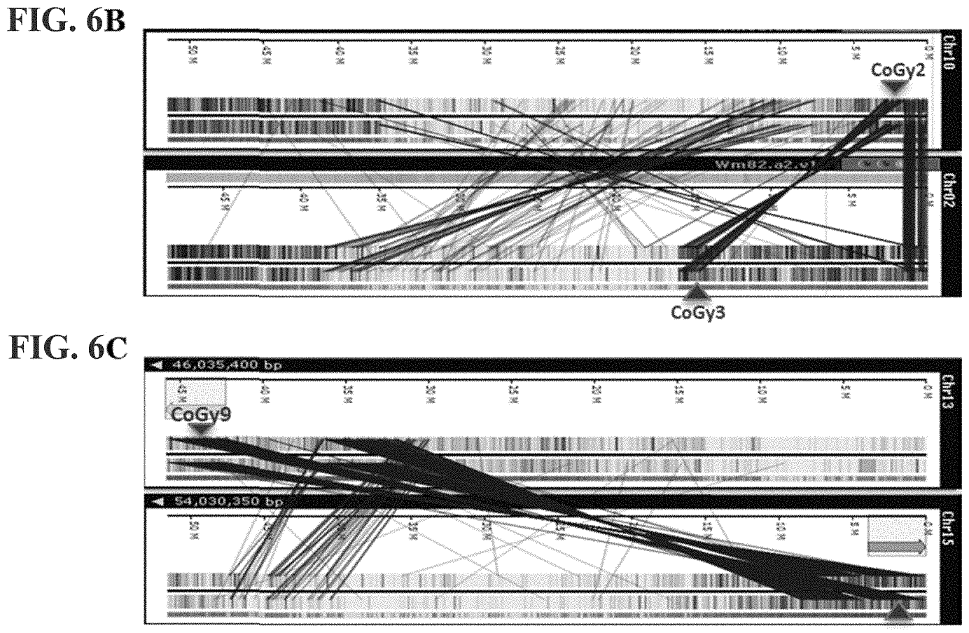

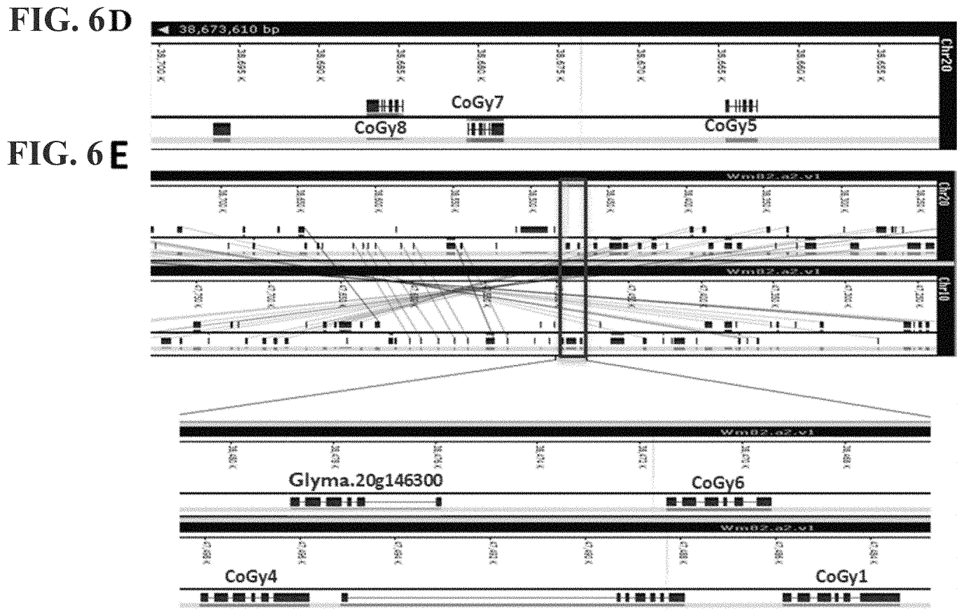

In order to test the contribution of the soybean duplication events in the number of β-ConGlycinin genes, the soybean genome was analyzed for duplicated chromosomal segments containing β-ConGlycinin. Syntenic analysis revealed the presence of two different tandem duplications within chr10 involving GmCoGy1 (Glyma.10g246300) and GmCoGy4 (Glyma.10g246500), in addition to the presence of another tandem duplication within chr20 involving GmCoGy5 (Glyma.20g148200), GmCoGy7 (Glyma.20g148300), and GmCoGy8 (Glyma.20g148400) (see FIG. 6(a)-6(e)). The syntenic relationships were calculated using the Persephone 4.3 software.

The other four GmCoGy genes belong to two different segment duplications. A segment duplication (−1.4 Mb) was found between chr02 and chr10 involving GmCoGy2 (Glyma.10g028300) and GmCoGy3 (Glyma.02g145700) and containing 250 conserved duplicated genes or anchors (see FIG. 6(a)-6(e)). The second segment duplication (˜3.5 Mb) between chr13 and chr15 involves GmCoGy9 (Glyma.13G328400) and another gene Glyma.02g145700 (carrying 1 Cupin domain only unlike the nine GmCoGy gene members) and containing 759 conserved duplicated genes or anchors (see FIG. 6(a)-6(e)). Although in silico analysis revealed the presence of two cupin domains for GmCoGy9 and GmCoGy6 members, their two duplicated members at chromosome 15 (Glyma.15g045300) and chromosome 20 (Glyma.20g146300) carry 1 cupin domain only. Investigation on the soybase database reveals the absence of any expression data for both genes in Williams 82. In Forrest, RNAseq data shows that Glyma.20g146300 and Glyma.15g045300 were not expressed in seeds, roots, leaves, flower and pods, with a very little expression (˜1 RKM for Glyma.20g146300 in pod and ˜1 RKM for Glyma.15g045300 in seed). Taken together, these data point to the presence of natural spontaneous occurring mutations at both the promoter affecting their gene expression and coding sequence affecting their first cupin domain during the soybean genome evolution creating enough diversion and selecting pressure affecting its molecular function. The absence of seed expression for GmCoGy9, Glyma.20g146300, and Glyma.15g045300 may explain their divergence observed on the phylogenetic analysis. The observed expression of the GmCoGy9 in pod only may point to a role that can play this β-ConGlycinin member in pod protein storage in soybeans.

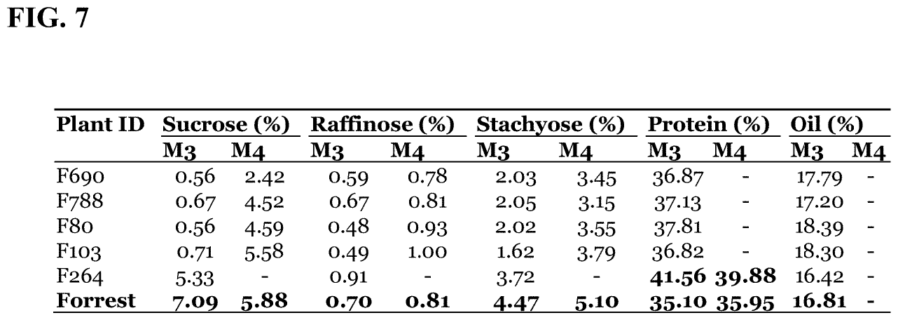

Phylogenetic analysis from the 21 sequenced plant species supported the synteny analysis. In fact, the GmCoGy2 (Glyma.10g028300)/GmCoGy3 (Glyma.02g145700), GmCoGy1 (Glyma.20g148300)/GmCoGy8 (Glyma.20g148400), and CoGy9/Glyma.15g045300 duplicated genes were separately grouped in three different sub-clades (see FIG. 7).

### Example 11. Identification of an EMS Mutant by Forward Genetics with Increased Proteins and Steady Oil Content

A forward genetics approach was used to produce soybean lines with increased proteins and steady or increased oil contents. It relies on the production of saturated mutant's soybean lines. In this study, a combined population of 4,032 soybean M2 and M3 lines were developed from which M3 seeds from each mutant line were screened for their protein content.

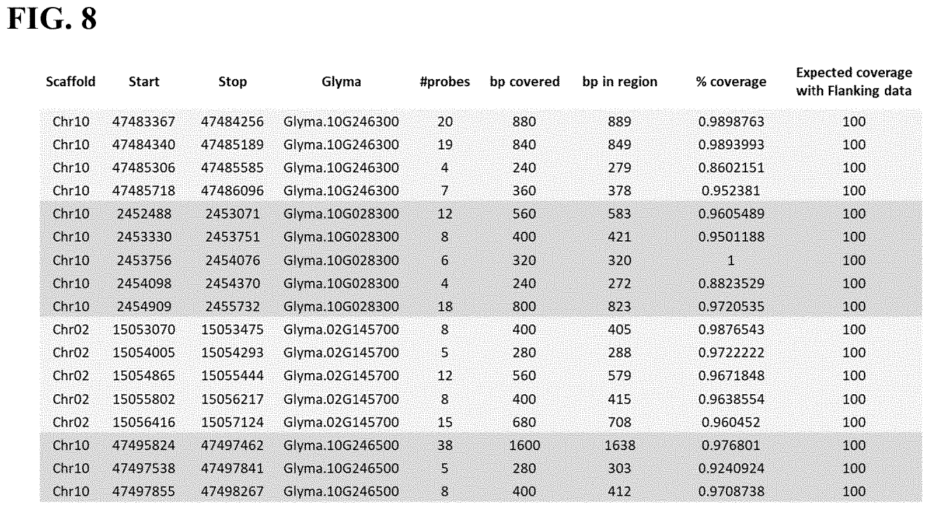

Although we noted the existence of a negative correlation between protein and oil content in soybean seeds, we were able to identify a high protein (41.56%) mutant line (F264) while maintaining a steady oil content similar to the wild-type oil (16.42%). Additionally, the mutant line, F264, with elevated protein content (10.9% higher than wild-type) was successfully advanced to the M3 and M4 generation preserving its high protein content heritability and steady oil compared to the wild type (see FIG. 8). These data strongly supported the possibility of isolating more EMS mutants containing high protein content without affecting the seed oil content in the soybean EMS mutant population. Thus, the use of a robust reverse genetic approach like TILLING-by-Sequencing⁺ present a nice alternative to understanding the role of the protein storage in soybean in controlling seed protein content and amino acid composition.

### Example 12. The Use of TILLING by Target Capture Sequencing as Reverse Genetics Approach to Identify Mutants within the β-ConGlycinin Gene Family

Previous studies have shown that high-protein soybean lines appear to contain more β-conglycinin and glycinin than normal-protein soybean lines, and the amounts of subunits and polypeptides differ among lines. Interestingly, it has been shown that mutations in the glycinin genes designated as Gy1, Gy2, Gy3, Gy4 and Gy5 have a direct impact on β-conglycinin content in Soybean seeds. Mutant soybean plants with decreased glycinin content resulted in increased β-conglycinin content. Therefore, the current study focuses on the manipulation of several genes belonging to the β-conglycinin gene family members to increase the total protein content of soybean. Mutagenesis of soybean seeds with EMS has been carried out to introduce mutations within the different subunits (building blocks) of the seed storage proteins β-conglycinin. FIG. 4 summarizes all soybean genes encoding for β-conglycinin subunits that were targeted in the current study.

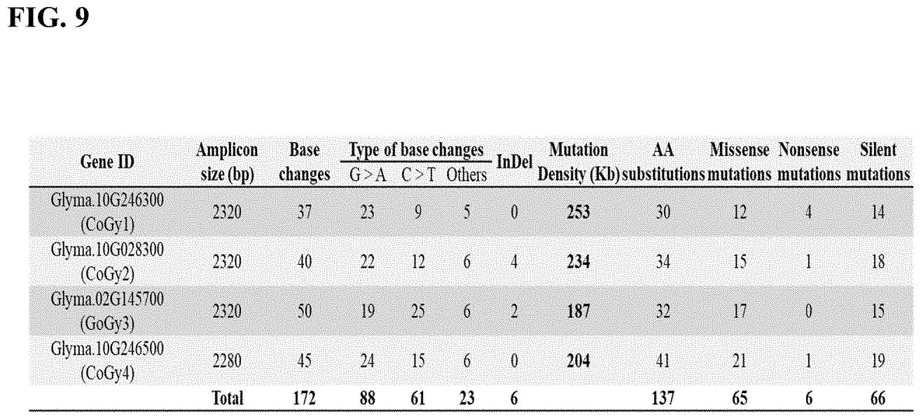

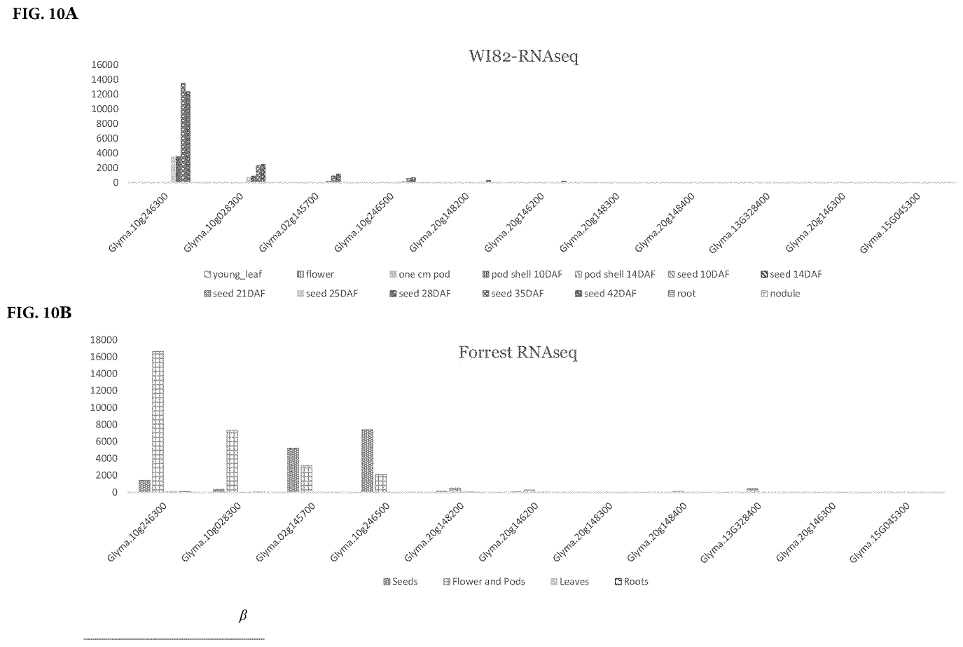

Recently, we developed a high-throughput TILLING-by-Sequencing⁺ (TbyS⁺) technology coupled with universal bioinformatic tools to identify population-wide mutations in soybeans. To identify mutants within the β-ConGlycinin gene family, 4,032 EMS mutagenized soybeans population was developed using the “Forrest” cultivar. Next, several gene-specific probes were designed for the TbyS⁺ platform (see FIG. 9), then capture enrichment technology was used to target the four highly expressed genes in soybean seeds including GmCoGy1, GmCoGy2, GmCoGy3, and GmCoGy4 (see FIGS. 10A and 10B). FIG. 10A shows the results for Williams 82 and FIG. 10B shows the results for Forrest cultivars. In Williams 82, GmCoGy1, GmCoGy2, GmCoGy3, and GmCoGy4 were at least ˜154, 28, 8, and 6-fold change highly expressed in seeds than the rest of the other five ConGlycinin members, respectively (FIGS. 4 and 10A and 10B). In Forrest, transcripts from GmCoGy1, GmCoGy2, GmCoGy3, and GmCoGy4 were at least ˜12, 3, 47, and 33-fold change more abundant than the other five ConGlycinin members, respectively. GmCoGy9 was not expressed in any of the plant tissue analyzed.

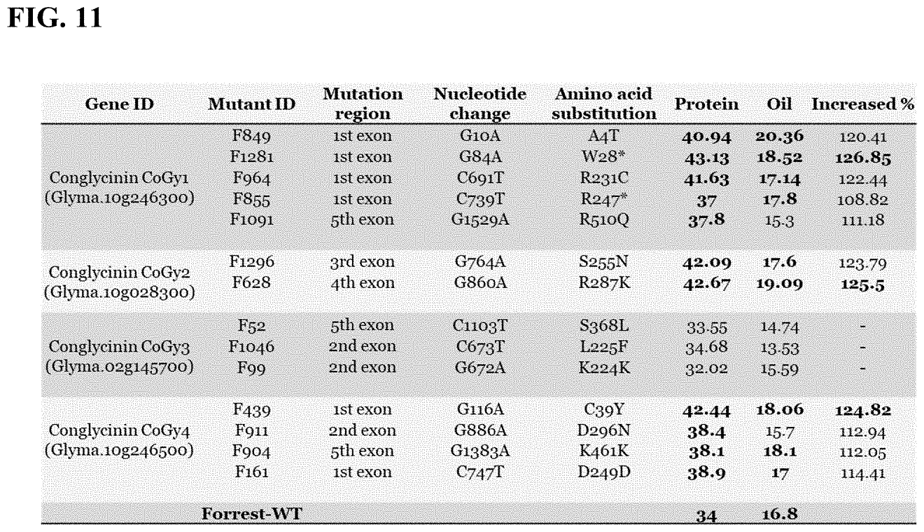

Using TbyS⁺ technology, we successfully identified several CoGy mutants. Three GmCoGy1 missense mutants (GmCoGy1_{A4T}, GmCoGy1_{R231C}, and GmCoGy1_{R510Q}), two GmCoGy1 nonsense mutants GmCoGy1_{W28*} (with an early premature stop codon) and GmCoGy1_{R247*}, two GmCoGy2 missense mutants GmCoGy2_{S255N} and GmCoGy2_{R287K}, three GmCoGy3 missense mutants (GmCoGy3_{S368L}, GmCoGy3_{L255F}, and GmCoGy3E_{100K}), two GmCoGy4 missense mutants (GmCoGy4_{C39Y} and GmCoGy4_{D296N}), and two silent GmCoGy4 mutants (GmCoGy4_{K461K} and GmCoGy4_{D249D}) were identified and selected for further functional characterization based on seed availability (see FIG. 11).

Structural analysis reveals that the β-ConGlycinin missense mutations were introduced in important locations on the protein sequence. Six β-ConGlycinin mutations were present in the first cupin domain, three in the second cupin domain, while one mutation (GmCoGy1_{A4T}) was located in a signal peptide (see FIG. 2). The first Cupin domain is located between 270 to 404 residues and the second Cupin domain is located between 637 to 760 residues. In silico analysis shows that the GmCoGy3 gene has an altered signal peptide sequence which is preceded by a 17 amino acid insertion. The 14 rectangles show the 14 TILLING mutations identified by TbyS⁺ in the four screened β-ConGlycinin members. 12 mutations and two nonsense mutations; GmCoGy1_{A4T}, GmCoGy1_{W28*}, GmCoGy1_{R231C}, GmCoGy1_{R247*}, GmCoGy1_{R510Q}, GmCoGy2_{S255N}, GmCoGy2_{K287K}, GmCoGy3_{S368L}, GmCoGy3_{L225F}, GmCoGy3_{K224K}, GmCoGy4_{C39Y}, GmCoGy4_{D296N}, GmCoGy4_{K461C}, and GmCoGy4_{D249D} are shown in FIG. 2. Six missense mutations are present in the first Cupin domain, three in the second cupin domain, and one mutation (GmCoGy1_{A4T}) was located in a signal peptide. Glyma.20g146300 and Glyma.15G045300 genes lack the first Cupin domain. Conserved domains were retrieved from Standard databases using Jalview software.

### Example 13. Mutation Density of the Forrest EMS-Mutagenized Soybean Population

Based on the Tilling-by-Sequencing⁺ analysis in the four β-ConGlycinin genes, we identified 172 single nucleotide polymorphism (SNP) mutations and six indels (see FIG. 8). Of the mutations, 86.6% were the typical EMS mutations (G to A and C to T), and the remaining 13.3% of mutations were accounted for by other types of mutations. The mutation density was estimated to be ˜ 1/253 kb, ˜ 1/234 kb, ˜ 1/187 kb, and ˜ 1/204 kb for CoGy1, CoGy2, CoGy3, and CoGy4 genes, respectively. Within the coding regions, a total of 65 missense mutations, six nonsense mutations, and 66 silent mutations were obtained (Table 2). The obtained mutation density is coherent with previous studies using either EMS or N-nitroso N-methylurea.

### Example 14. Protein Homology Modeling and Mutational Analysis Reveals Unique Features of the β-ConGlycinin Homo-Trimer

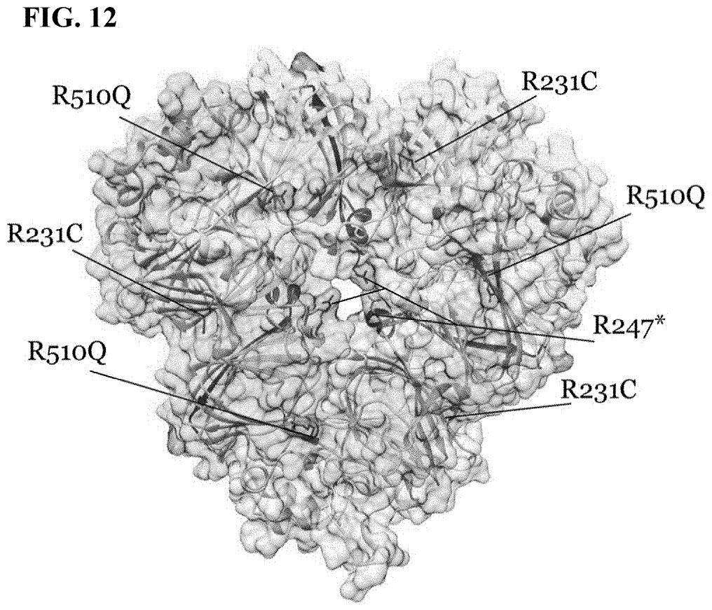

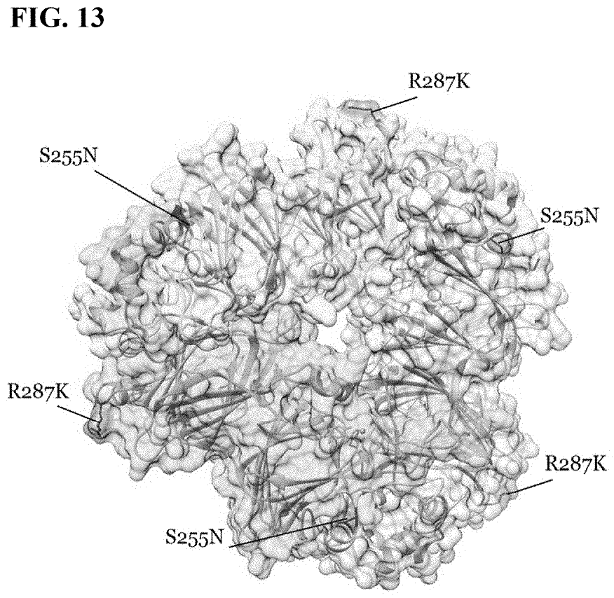

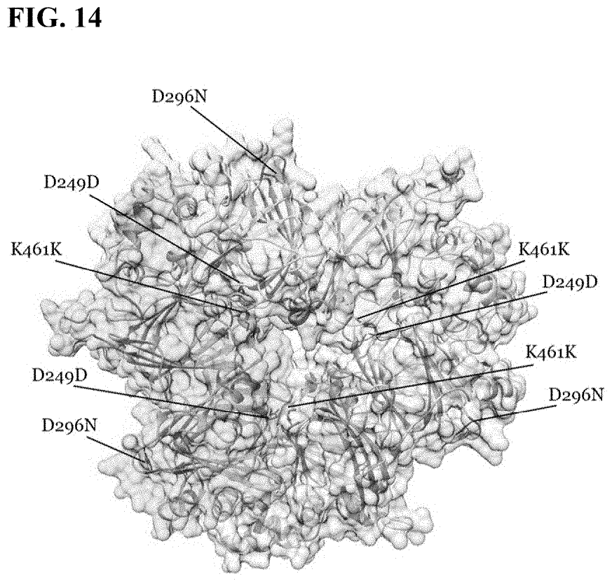

β-Conglycinin is a glycoprotein and a trimer which consists of three subunits α′, α, and β with a molecular mass of 150-200 kDa. The molecular weights of the three major subunits are 72, 68, and 52 kDa, respectively. To gain more insight into the role of the identified missense mutations and their impact on the protein structure, homology modeling of the GmCoGy proteins and mutational analysis have been carried out. The identified missense mutations within the β-ConGlycinins GmCoGy1 (see FIG. 12), GmCoGy2 (see FIG. 13), and GmCoGy4 (see FIG. 14) proteins were mapped in key positions that impact either the dimerization or trimerization of the GmCoGy proteins. The identified CoGy1_{R247*} mutation was mapped in key position that impact the trimerization of the three β-ConGlycinin CoGy1 subunits. The CoGy1_{W28*} nonsense mutations resulted in a premature stop codon and therefore this truncation may impact the formation of the β-ConGlycinin CoGy1 trimer, resulting in an increased protein content in soybean seeds. The three different subunits constituting the β-ConGlycinin CoGy1 trimer are highlighted in FIG. 12. Most importantly, the CoGy1_{R231C} and CoGy1_{R510Q} mutations were mapped very close to the predicted allergen peptide “PRRHKNKNPFHFNSKRFQ” (SEQ ID NO: 51), which is similar to the reported peptide-containing the epitope “LRRHKNKNPFLFGSNRFE” (SEQ ID NO: 52) in the amino acid sequence of Gly m 5 β-conglycinin α-subunit. Soybean allergen peptides are shown (KNPQLRDLDVFLSVVDMNE) (SEQ ID NO: 53) and (PRRHKNKNPFHFNSKRFQ) (SEQ ID NO: 51).

In FIG. 13, the two identified CoGy2_{S255N} and CoGy2_{R287K} mutations were mapped in key positions that impact the dimerization of the β-ConGlycinin CoGy2 subunits. Mutations in these residues resulted in increased protein content in soybean seeds. The three different subunits constituting the β-ConGlycinin CoGy2 trimer are highlighted in FIG. 13.

The three identified CoGy4_{D296N}, CoGy4_{K461K}, and CoGy4_{D249D} mutations may also impact the dimerization of the β-ConGlycinin CoGy2 subunits. Interestingly, in FIG. 14, both CoGy4_{K461K} and CoGy4_{D249D} mutations were mapped in key positions that impact the dimerization of the β-ConGlycinin CoGy2 subunits, and also were mapped very close to the predicted allergen peptide “KNPQLRDFDILLNTVDINE” (SEQ ID NO: 54), which is similar to the reported peptide-containing epitope “KNPQLRDLDIFLSIVDMNE” (SEQ ID NO: 55) in the amino acid sequence of Gly m 5 β-conglycinin α-subunit. These mutations resulted in increased protein content in soybean seeds. The three different subunits constituting the β-ConGlycinin CoGy2 trimers are highlighted in FIG. 14.

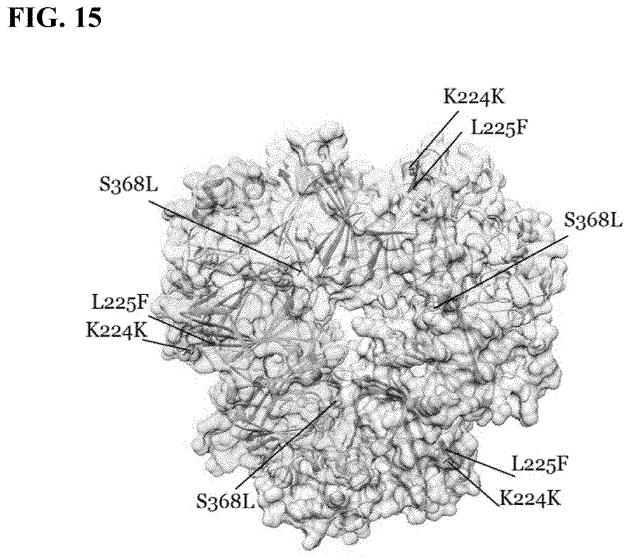

In the case of GmCoGy3 mutants, protein homology modeling reveals that GmCoGy3_{K224K}, GmCoGy3_{L225F}, and GmCoGy3_{S368L} mutations were mapped outside the dimerization and/or trimerization sites (see FIG. 15), which may explain the observed normal protein phenotype in this mutant. The silent GmCoGy3_{K224K} mutation may also result in non-deleterious effect on the GmCoGy3 protein enzymatic activity. These data may point to either the absence of functional redundancy of the GmCoGy3 gene member in increasing seed protein content or may be explain by the absence of deleterious mutations that may affect the GmCoGy3 protein activity at these three identified residues.

In order to test the contribution of the four β-ConGlycinin genes in the seed protein and amino acid composition, the identified EMS β-ConGlycinin mutants were phenotyped for their seed protein content. Interestingly, with the exception of the CoGy3 mutants, all CoGy1, CoGy2, and CoGy4 EMS mutants showed a significant increase in their protein content. The nonsense mutation GmCoGy1_{W28*} resulted in the highest protein content increase (up 43.13%) (see FIG. 11). Mutations at the GmCoGy2 resulted also in a protein increase up to 42.67% (CoGy2_{S255N}). Similarly, mutations at the GmCoGy4 increased protein content up to 42.44% (CoGy4_{C39Y}). Notably, mutations on β-ConGlycinin1, β-ConGlycinin2, and β-ConGlycinin4 increased protein content by 26.85%, 25.5%, and 24.82%, respectively, when compared to the Forrest wild type (see FIG. 11). Surprisingly, mutations in the β-ConGlycinin also resulted in a significant increase of oil content from 16.8% contained in the Forrest WT up to 20.36% (CoGy1_{A4T}), which is ˜21% increase when compared to the Forrest wild type (see FIG. 11). This may explain the effect of the isolated missense mutations that negatively impacted the formation of the β-ConGlycinin homo-trimer, which may have negatively impacted their enzymatic activity. This impact might decrease the level of seed β-ConGlycinin, which may increase the presence of free amino acids by redirecting the carbon flux. Redirecting the carbon flux in soybean seeds may be the reason why total protein and oil content increases in soybean seeds.

This is the first report showing that induced β-ConGlycinin mutations within the alpha subunits (CoGy1 and CoGy4) and the sucrose binding subunits (CoGy2) resulted in increased protein and oil content in soybean seeds.

### Example 15. Mutations at the β-ConGlycinin Resulted in a Change on Protein Profile and Amino Acid Composition

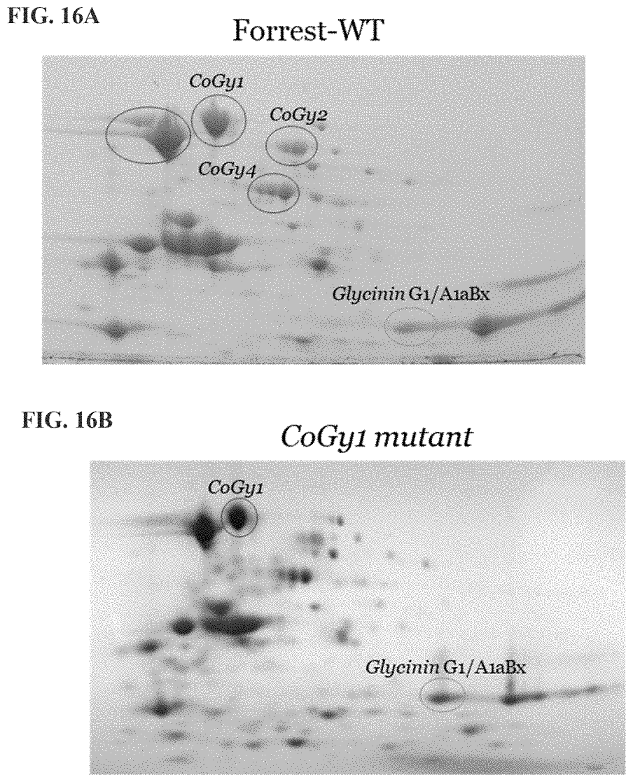

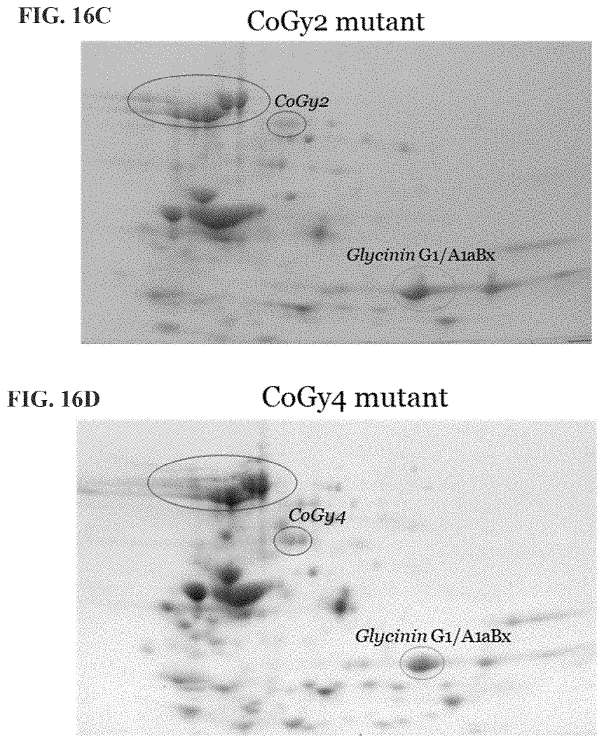

Soybean crude extract from the Forrest-WT and TILLING mutants was subjected to 2D-PAGE followed by Coomassie Blue staining. Gel staining revealed the presence of a wide number of proteins that were gained on the seed profile of the TILLING mutants when compared to the Forrest-WT. Mutations at the three β-ConGlycinin CoGy1, CoGy2, and CoGy4 resulted in decreased β-ConGlycinin but increasing glycinin as shown on the 2D-PAGE (see FIGS. 16A, 16B, 16C and 16D).

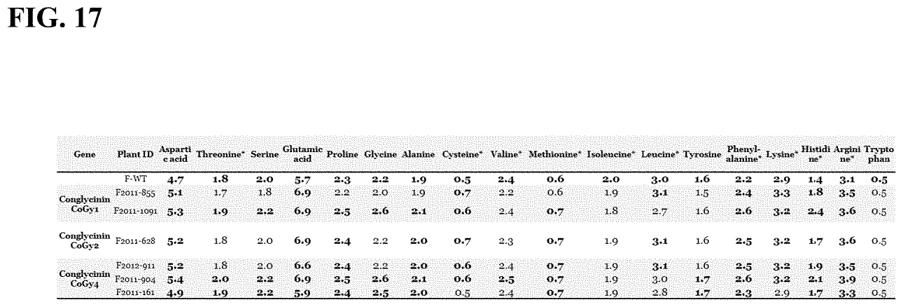

Next, the amino acid profile including all essential amino acids have been tested on the CoGy1, CoGy2, and CoGy4 mutants. As shown in FIG. 17, CoGy1, CoGy2, and CoGy4 mutations have a positive impact increasing essential amino acid specially cysteine (up to 40%), methionine (up to 16%), Phenyl-alanine (up to 18%), Lysine (up to 13%), Histidine (up to 71%), and Arginine (up to 25%). Non-essential amino acids including aspartic acid, glutamic acid, proline, glycine, and alanine have also increased significantly on the β-ConGlycinin mutants up to 14%, 21%, 8%, 18%, and 10%, respectively.

The use of this technology created a positive impact on decreasing the amount of the conglycinin protein profile, redirecting the carbon flux to improve the amino acid composition and protein content up to 43% in soybeans.

### Example 16. Soybean Lines with Elevated Protein and Oil Content

Soybean is a complete plant protein with several benefits for human and animals. Soybean is a primary source of nutrition in high-quality feed for animal nutrition and is considered the ideal direct source of protein for people as a readily available and sustainably produced protein. The global protein ingredients market size is projected to grow from USD 49.8 billion in 2019 to USD 70.7 billion by 2025 (at a CAGR of 6.0% during the forecast period). Soybean is a complete plant protein with several benefits for human and animals. Soybean is a primary source of nutrition in high-quality feed for animal nutrition and is considered the ideal direct source of protein for people as a readily available and sustainably produced protein. Up to date, limited knowledge about the use of protein storage in soybeans is available. A recent study reported the mobilization of storage proteins in soybean seed (Glycine max L.) during germination and seedling early growth by proteases

Glycinin and β-ConGlycinin are the two primary classes of seed storage proteins. About 90-95% of the soy is a storage protein, where the two subunits; conglycinin (7S) and glycinin (11S) constitute about 35% and 52% of the seed total protein, respectively. Both soybean storage protein structures are highly conserved to maximize protein packaging in the protein bodies. Considerable efforts have been dedicated to characterize the Glycinin genes but little is known about the β-ConGlycinin gene family in soybean. β-ConGlycinin is a trimeric protein composed of a few subunits. The genes responsible of synthesizing the different subunits are divided into two groups that are similar including α-subunits and β-subunits. However, most of the β-ConGlycinin gene family members are unexplored.

Twenty years ago, it has been reported that co-suppression of the α-subunit of β-conglycinin in transgenic soybean seeds induces the formation of endoplasmic reticulum-derived protein bodies but the transgenic seeds had similar total oil and protein content and ratio compared to the parent line. The decrease in β-conglycinin protein in these transgenic soybeans was compensated by an increased accumulation of glycinin. In addition, proglycinin, the precursor of glycinin, was detected as a prominent polypeptide band in the protein profile of the transgenic seed extract. Coherent results were obtained from the current study using EMS-mutagenesis when reducing β-conglycinin to increase glycinin and the rest of seed amino acids in soybeans.

Although soybean yield and oil content were increasing in the US as a result of intense soybean breeding programs, the average of soybean seed protein content was decreasing. Environmental stress such us drought and heavy rain negatively impacts soybean nutritional composition resulting in a reduction in growth. During the last years, some protein QTLs have been identified, however, lack of understanding the genetic mechanism and identification of key genes involved in protein content that are responsible for protein synthesis and storage is the major issue of the protein industry. Additionally, most of the developed soybeans with relatively high protein content were affected in their oil composition and presented non-stable protein contents. It's well known that the regulation of carbon flux during embryogenesis might be shifted toward either protein or oil biosynthesis, which is impacted by both genetics and environment. Microenvironments were reported to impact carbon flux during embryogenesis, where pods situated at the top of the plant having seeds with a higher percentage of protein and lower oil content when compared to those located at the bottom of the plant. Although soybean breeders have improved soybean yield that was translated in more protein per acre, limited progress have been achieved to selecting high yielding genotypes with considerable shifts in carbon flux to improve total oil and protein. Although a negative correlation has been reported between protein and oil from numerous breeding programs using different soybean germplasms (natural genetic diversity), a recent study has shown the possibility of increasing both traits when using EMS induced mutagenesis (˜40% protein and 20.7% oil). The use of advance molecular biology and biotechnology techniques has launched the development of improved end-use quality of the oil for food, feed, and industrial applications. Best example is the modification of fatty acid biosynthesis to alter relative amounts of healthy beneficial fatty acids in soybean or to produce novel fatty acids. Nutritional enhancement of soybean has emphasized improving their protein levels for food and feed applications as well. This study used the TILLING-by-sequencing⁺ technology to identify and characterize four highly expressed members of the β-ConGlycinin gene family based on their seed expression analysis from the soybean reference genome Williams 82 and Forrest cultivars, study their role in increasing soybean seed protein content and amino acid composition, in addition to developing sources of non-GMO soybean germplasms with increased soybean seed protein concentration while maintaining oil content.

Most importantly, the current study showed that most of the identified mutations at the CoGy1, CoGy2, and CoGy4 were located very close to the dimerization or trimerization sites between the three subunits constituting the β-ConGlycinin homo-di-trimer structure, which may impact negatively the trimerization of the β-ConGlycinin proteins. This is coherent with the 2D-PAGE analysis showing the decrease of the β-ConGlycinin band intensity on the β-ConGlycinin mutants which affected positively the total protein profile by increasing the amino acid composition in these mutants. Thus, producing deleterious mutations in one of the subunits of β-ConGlycinin may lead to non-assembly or poor assembly of the whole protein, which may create an opportunity for redirecting the carbon flux in soybean toward producing lines with increased proteins other than the major one (β-ConGlycinin), which positively impacted the amino acid composition and total oil content in soybean. Unlike previous reports suggesting the absence of strong metabolic links between oil and storage protein synthesis, the discovery from the current study suggest the presence of links between β-ConGlycinin and oil biosynthesis. Disrupting β-ConGlycinin protein biosynthesis resulted in increasing protein and oil content.

Unlike P1605781 B containing natural spontaneous mutations in both Gy4 and Gy1 glycinin genes exhibiting reduced glycinin content but unchanged total seed protein content, the current study has shown that single mutations at least in three β-ConGlycinin members resulted in increased protein content. Although silent mutations may not have an effect on the protein function, it has been shown that many silent mutations can have impact on the protein function. For example, the GmFAD2-1A_{L249L} silent mutation affected dramatically the GmFAD2-1A protein enzyme activity resulting in a drastic accumulation of the oleic acid content from ˜18% to >47% in soybean seeds. Similarly, in the current study, two silent mutations resulted in increased protein content and amino acid composition. The observed phenotype on CoGy4_{K461K} may be due to the affected soybean codon usage/frequency from 57.5% (AAG) to 42.5% (AAA) at the G1383A position. The other silent mutation CoGy4D249D may be due to the presence of background mutations on the F161 mutant.

Additionally, the presence of cross-reactive epitopes between bovine α-casein and soy β-conglycinin has been reported. Exposure to soybean proteins has become relevant in milk allergic pediatric patients due to cross allergenicity described between soy and milk proteins. It has been shown that Gly-m5 β-conglycinin α-subunit contain three peptide-containing epitopes in its amino acid sequence, including the “LRRHKNKNPFLFGSNRFE” (SEQ ID NO: 52). We were able to identify the presence of several similar peptide-containing epitopes on the β-ConGlycinin CoGy1 and CoGy4 amino acid sequence that are predicted to have similar soybean allergen properties. Interestingly, CoGy1_{R231C} and CoGy1_{R510Q} mutations were mapped very close to the predicted allergen peptide “PRRHKNKNPFHFNSKRFQ” (SEQ ID NO: 51), while the two identified CoGy4_{K461K} and CoGy4_{D249D} mutations were mapped very close to the predicted allergen peptide “KNPQLRDFDILLNTVDINE” (SEQ ID NO: 54). Therefore, the characterized missense mutants present several features including high protein and oil content, in addition to having additional benefits that may reduce soybean seed allergens.

The current study provides insight into the molecular function of the β-conglycinin protein family members, the origin and history of the genes, and their molecular function.

We demonstrated by using TILLING-by-Sequencing⁺ coupled with EMS mutagenesis to introduce random mutation in soybean the feasibility of producing soybean lines with high protein content, amino acid composition, and steady to high oil content in soybean seeds. The current study showed the feasibility to decrease the level of seed β-ConGlycinin by introducing point mutations on β-ConGlycinin CoGy1, CoGy2, and CoGy4 genes, which may increase the presence of free amino acids and redirect the carbon flux. Redirecting the carbon flux in soybean seeds may be the reason why total protein. Amino acids, and oil content increases in soybean seeds. The developed soybean seeds may have additional benefits in reducing soybean seed allergens, and therefore, might be beneficial for human and animal consumptions and health. The developed β-ConGlycinin lines from the current study will benefit soybean farmers and private industry for developing soybean lines with high protein content while maintaining their oil content.

1. A transgenic soybean plant having increased seed protein content and/or increased seed oil content comprising a polynucleotide encoding a β-ConGlycinin soybean seed storage promoter that functions in the soybean plant operably linked to a polynucleotide encoding a soybean seed storage polypeptide having β-ConGlycinin activity.

2. The plant of claim 1, wherein the polynucleotide encoding a β-ConGlycinin soybean seed storage promoter comprises a wild type β-Conglycinin promoter sequence, or a sequence at least 95% identical thereto, or a full length complement thereof, or a functional fragment thereof.

3. The plant of claim 2, wherein the wild type β-Conglycinin promoter sequence is selected from the group consisting of a β-Conglycinin CoGy1 promoter sequence, a β-Conglycinin CoGy2 promoter sequence, a β-Conglycinin CoGy3 promoter sequence, and a β-Conglycinin CoGy4 promoter sequence.

4. The plant of claim 1, wherein the polynucleotide encoding a soybean seed storage polypeptide having β-ConGlycinin activity comprises a wild type β-Conglycinin genomic or coding sequence, or a sequence at least 95% identical thereto, or a full length complement thereof, or a functional fragment thereof.

5. The plant of claim 4, wherein the wild type β-Conglycinin genomic or coding sequence is selected from the group consisting of a CoGy1 genomic (SEQ ID NO: 58) or coding (SEQ ID NO: 59) sequence, CoGy2 genomic (SEQ ID NO: 62) or coding (SEQ ID NO: 63) sequence, CoGy3 genomic (SEQ ID NO: 66) or coding (SEQ ID NO: 67) sequence, CoGy4 genomic (SEQ ID NO: 70) or coding (SEQ ID NO: 71) sequence.

6. The plant of claim 1, wherein the polynucleotide encoding the β-ConGlycinin soybean seed storage promoter comprises a wild type “Williams 82” β-Conglycinin or “Forrest” 3-Conglycinin promoter sequence, or a sequence at least 95% identical thereto, or a full-length complement thereof, or a functional fragment thereof.

7. The plant of claim 6, wherein the wild type “Williams 82” β-Conglycinin promoter sequence is selected from the group consisting of a wild type “Williams 82” β-Conglycinin CoGy1 promoter sequence (SEQ ID NO: 56), a wild type “Williams 82” β-Conglycinin CoGy2 promoter sequence (SEQ ID NO: 60), a wild type “Williams 82” β-Conglycinin CoGy3 promoter sequence (SEQ ID NO: 64), and a wild type “Williams 82” β-Conglycinin CoGy4 promoter sequence (SEQ ID NO: 68); or
wherein the wild type “Forrest” β-Conglycinin promoter sequence is selected from the group consisting of a wild type “Forrest” β-Conglycinin CoGy1 promoter sequence, a wild type “Forrest” β-Conglycinin CoGy2 promoter sequence, a wild type “Forrest” β-Conglycinin CoGy3 promoter sequence, and a wild type “Forrest” β-Conglycinin CoGy4 promoter sequence.

8. The plant of claim 1, wherein the polynucleotide encoding the soybean seed storage polypeptide having β-ConGlycinin activity comprises a wild type “Williams 82” β-Conglycinin or “Forrest” β-Conglycinin genomic or coding sequence, or a sequence at least 95% identical thereto, or a full-length complement thereof, or a functional fragment thereof, and optionally further comprises one or more mutations of the wild type “Williams 82” β-Conglycinin or “Forrest” β-Conglycinin genomic sequence.

9. The plant of claim 8, wherein the polynucleotide encoding the soybean seed storage polypeptide having β-ConGlycinin activity comprises a wild type “Williams 82” β-Conglycinin CoGy1 genomic (SEQ ID NO: 58) or coding (SEQ ID NO: 59) sequence, or a sequence at least 95% identical thereto, or a full-length complement thereof, or a functional fragment thereof, and optionally further comprises one or more mutations of the wild type “Williams 82” β-Conglycinin CoGy1 genomic sequence (SEQ ID NO: 58) selected from the group consisting of: G10A, G84A, C691T, C739T, and G1529A; or
wherein the polynucleotide encoding the soybean seed storage polypeptide having β-ConGlycinin activity comprises a wild type “Forrest” β-Conglycinin CoGy1 genomic or coding sequence, or a sequence at least 95% identical thereto, or a full-length complement thereof, or a functional fragment thereof, and optionally further comprises one or more mutations of the wild type “Forrest” β-Conglycinin CoGy1 genomic sequence selected from the group consisting of: G10A, G84A, C691T, C739T, and G1529A.

10. The plant of claim 8, wherein the polynucleotide encoding the soybean seed storage polypeptide having β-ConGlycinin activity comprises a wild type “Williams 82” β-Conglycinin CoGy2 genomic (SEQ ID NO: 62) or coding (SEQ ID NO: 63) sequence, or a sequence at least 95% identical thereto, or a full-length complement thereof, or a functional fragment thereof, and optionally further comprises one or more mutations of the wild type “Williams 82” β-Conglycinin CoGy2 genomic sequence (SEQ ID NO: 62) selected from the group consisting of: G764A and G860A; or
wherein the polynucleotide encoding the soybean seed storage polypeptide having (3-ConGlycinin activity comprises a wild type “Forrest” 3-Conglycinin CoGy2 genomic or coding sequence, or a sequence at least 95% identical thereto, or a full-length complement thereof, or a functional fragment thereof, and optionally further comprises one or more mutations of the wild type “Forrest” 3-Conglycinin CoGy2 genomic sequence selected from the group consisting of: G764A and G860A.

11. The plant of claim 8, wherein the polynucleotide encoding the soybean seed storage polypeptide having 3-ConGlycinin activity comprises a wild type “Williams 82” 3-Conglycinin CoGy3 genomic (SEQ ID NO: 66) or coding (SEQ ID NO: 67) sequence, or a sequence at least 95% identical thereto, or a full-length complement thereof, or a functional fragment thereof, and optionally further comprises one or more mutations of the wild type “Williams 82” 3-Conglycinin CoGy3 genomic sequence (SEQ ID NO: 66); or
wherein the polynucleotide encoding the soybean seed storage polypeptide having (3-ConGlycinin activity comprises a wild type “Forrest” 3-Conglycinin CoGy3 genomic or coding sequence, or a sequence at least 95% identical thereto, or a full-length complement thereof, or a functional fragment thereof, and optionally further comprises one or more mutations of the wild type “Forrest” 3-Conglycinin CoGy3 genomic sequence.

12. The plant of claim 8, wherein the polynucleotide encoding the soybean seed storage polypeptide having 3-ConGlycinin activity comprises a wild type “Williams 82” 3-Conglycinin CoGy4 genomic (SEQ ID NO: 70) or coding (SEQ ID NO: 71) sequence, or a sequence at least 95% identical thereto, or a full-length complement thereof, or a functional fragment thereof, and may further comprise one or more mutations of the wild type “Williams 82” 3-Conglycinin CoGy4 genomic sequence (SEQ ID NO: 70) selected from the group consisting of: G116A, C747T, G886A, and G1383A; or
wherein the polynucleotide encoding the soybean seed storage polypeptide having (3-ConGlycinin activity comprises a wild type “Forrest” 3-Conglycinin CoGy4 genomic or coding sequence, or a sequence at least 95% identical thereto, or a full-length complement thereof, or a functional fragment thereof, and may further comprise one or more mutations of the wild type “Forrest” β-Conglycinin CoGy4 genomic sequence selected from the group consisting of: G116A, C747T, G886A, and G1383A.

13. The plant of claim 1, wherein the soybean seed storage polypeptide having β-ConGlycinin activity comprises a wild type “Williams 82” β-Conglycinin or “Forrest” β-Conglycinin sequence, or a sequence at least 95% identical thereto, or a full-length complement thereof, or a functional fragment thereof, and optionally further comprises one or more mutations of the wild type “Williams 82” β-Conglycinin or “Forrest” β-Conglycinin sequence.

14. The plant of claim 13, wherein the soybean seed storage polypeptide having β-ConGlycinin activity comprises a wild type “Williams 82” β-Conglycinin CoGy1 sequence (SEQ ID NO: 57), or a sequence at least 95% identical thereto, or a full-length complement thereof, or a functional fragment thereof, and optionally further comprises one or more mutations of the wild type “Williams 82” β-Conglycinin CoGy1 sequence selected from the group consisting of: A4T, W28*, R231C, R247*, and R510Q; or
wherein the soybean seed storage polypeptide having β-ConGlycinin activity comprises a wild type “Forrest” β-Conglycinin CoGy1 sequence, or a sequence at least 95% identical thereto, or a full-length complement thereof, or a functional fragment thereof, and optionally further comprises one or more mutations of the wild type “Forrest” β-Conglycinin CoGy1 sequence selected from the group consisting of: A4T, W28*, R231C, R247*, and R510Q.

15. The plant of claim 13, wherein the soybean seed storage polypeptide having β-ConGlycinin activity comprises a wild type “Williams 82” β-Conglycinin CoGy2 sequence (SEQ ID NO: 61), or a sequence at least 95% identical thereto, or a full-length complement thereof, or a functional fragment thereof, and optionally further comprises one or more mutations of the wild type “Williams 82” β-Conglycinin CoGy2 sequence selected from the group consisting of: S255N and R287K; or
wherein the soybean seed storage polypeptide having β-ConGlycinin activity comprises a wild type “Forrest” β-Conglycinin CoGy2 sequence, or a sequence at least 95% identical thereto, or a full-length complement thereof, or a functional fragment thereof, and optionally further comprises one or more mutations of the wild type “Forrest” β-Conglycinin CoGy2 sequence selected from the group consisting of: S255N and R287K.

16. The plant of claim 13, wherein the soybean seed storage polypeptide having (3-ConGlycinin activity comprises a wild type “Williams 82” β-Conglycinin CoGy3 sequence (SEQ ID NO: 65), or a sequence at least 95% identical thereto, or a full-length complement thereof, or a functional fragment thereof, and optionally further comprises one or more mutations of the wild type “Williams 82” 3-Conglycinin CoGy3 sequence; or
wherein the soybean seed storage polypeptide having 3-ConGlycinin activity comprises a wild type “Forrest” 3-Conglycinin CoGy3 sequence, or a sequence at least 95% identical thereto, or a full-length complement thereof, or a functional fragment thereof, and optionally further comprises one or more mutations of the wild type “Forrest” 3-Conglycinin CoGy3 sequence.

17. The plant of claim 13, wherein the soybean seed storage polypeptide having (3-ConGlycinin activity comprises a wild type “Williams 82” 3-Conglycinin CoGy4 sequence (SEQ ID NO: 69), or a sequence at least 95% identical thereto, or a full-length complement thereof, or a functional fragment thereof, and optionally further comprises one or more mutations of the wild type “Williams 82” 3-Conglycinin CoGy4 sequence selected from the group consisting of: C39Y, D249D, D296N, and K461K; or
wherein the soybean seed storage polypeptide having 3-ConGlycinin activity comprises a wild type “Forrest” 3-Conglycinin CoGy4 sequence, or a sequence at least 95% identical thereto, or a full-length complement thereof, or a functional fragment thereof, and optionally further comprises one or more mutations of the wild type “Forrest” 3-Conglycinin CoGy4 sequence selected from the group consisting of: C39Y, D249D, D296N, and K461K.

18. The plant of claim 1, wherein the increased seed protein content represents an at least about 1%, at least about 2%, at least about 3%, at least about 4%, at least about 5%, at least about 6%, at least about 7%, at least about 8%, at least about 9%, at least about 10%, at least about 20%, at least about 30%, at least about 40%, at least about 50%, at least about 60%, at least about 70%, at least about 80%, at least about 90%, or at least about 100% increase in seed protein content compared to a control soybean plant lacking the polynucleotide encoding a 3-ConGlycinin soybean seed storage polynucleotide.

19. The plant of claim 1, wherein the increased seed oil content represents an at least about 1%, at least about 2%, at least about 3%, at least about 4%, at least about 5%, at least about 6%, at least about 7%, at least about 8%, at least about 9%, at least about 10%, at least about 20%, at least about 30%, at least about 40%, at least about 50%, at least about 60%, at least about 70%, at least about 80%, at least about 90%, or at least about 100% increase in seed oil content as compared to a control soybean plant lacking the polynucleotide encoding a β-ConGlycinin soybean seed storage polynucleotide.

20. A method of increasing seed protein content and/or increasing seed oil content of a soybean plant, the method comprising:
transforming the soybean plant with a polynucleotide encoding a β-ConGlycinin soybean seed storage promoter that functions in the soybean plant operably linked to a polynucleotide encoding a soybean seed storage polypeptide having β-ConGlycinin activity.
